SEQUENCE LISTING (17 sequences)

SEQ ID NO: 1 - DNA, 623 bp, Artificial Sequence
  Recombinant synthesis
aattctgtcattttactagggtgatgaaattcccaagcaacaccatccttttcagataagggcactgaggctgagagaggagctgaaacctacccggggtcaccacacacaggtggcaaggctgggaccagaaaccaggactgttgactgcagcccggtattcattctttccatagcccacagggctgtcaaagaccccagggcctagtcagaggctcctccttcctggagagttcctggcacagaagttgaagctcagcacagccccctaacccccaactctctctgcaaggcctcaggggtcagaacactggtggagcagatcctttagcctctggattttagggccatggtagagggggtgttgccctaaattccagccctggtctcagcccaacaccctccaagaagaaattagaggggccatggccaggctgtgctagccgttgcttctgagcagattacaagaagggactaagacaaggactcctttgtggaggtcctggcttagggagtcaagtgacggcggctcagcactcacgtgggcagtgccagcctctaagagtgggcaggggcactggccacagagtcccagggagtcccaccagcctagtcgccagacc

SEQ ID NO: 2 - DNA, 941 bp, Artificial Sequence
  Recombinant synthesis
ccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattggatc

SEQ ID NO: 3 - DNA, 1758 bp, Homo sapiens
atgaccatcacttacacaagccaagtggctaatgcccgcttaggctccttctcccgcctgctgctgtgctggcggggcagcatctacaagctgctatatggcgagttcttaatcttcctgctctgctactacatcatccgctttatttataggctggccctcacggaagaacaacagctgatgtttgagaaactgactctgtattgcgacagctacatccagctcatccccatttccttcgtgctgggcttctacgtgacgctggtcgtgacccgctggtggaaccagtacgagaacctgccgtggcccgaccgcctcatgagcctggtgtcgggcttcgtcgaaggcaaggacgagcaaggccggctgctgcggcgcacgctcatccgctacgccaacctgggcaacgtgctcatcctgcgcagcgtcagcaccgcagtctacaagcgcttccccagcgcccagcacctggtgcaagcaggctttatgactccggcagaacacaagcagttggagaaactgagcctaccacacaacatgttctgggtgccctgggtgtggtttgccaacctgtcaatgaaggcgtggcttggaggtcgaatccgggaccctatcctgctccagagcctgctgaacgagatgaacaccttgcgtactcagtgtggacacctgtatgcctacgactggattagtatcccactggtgtatacacaggtggtgactgtggcggtgtacagcttcttcctgacttgtctagttgggcggcagtttctgaacccagccaaggcctaccctggccatgagctggacctcgttgtgcccgtcttcacgttcctgcagttcttcttctatgttggctggctgaaggtggcagagcagctcatcaacccctttggagaggatgatgatgattttgagaccaactggattgtcgacaggaatttgcaggtgtccctgttggctgtggatgagatgcaccaggacctgcctcggatggagccggacatgtactggaataagcccgagccacagcccccctacacagctgcttccgcccagttccgtcgagcctcctttatgggctccaccttcaacatcagcctgaacaaagaggagatggagttccagcccaatcaggaggacgaggaggatgctcacgctggcatcattggccgcttcctaggcctgcagtcccatgatcaccatcctcccagggcaaactcaaggaccaaactactgtggcccaagagggaatcccttctccacgagggcctgcccaaaaaccacaaggcagccaaacagaacgttaggggccaggaagacaacaaggcctggaagcttaaggctgtggacgccttcaagtctgccccactgtatcagaggccaggctactacagtgccccacagacgcccctcagccccactcccatgttcttccccctagaaccatcagcgccgtcaaagcttcacagtgtcacaggcatagacaccaaagacaaaagcttaaagactgtgagttctggggccaagaaaagttttgaattgctctcagagagcgatggggccttgatggagcacccagaagtatctcaagtgaggaggaaaactgtggagtttaacctgacggatatgccagagatccccgaaaatcacctcaaagaacctttggaacaatcaccaaccaacatacacactacactcaaagatcacatggatccttattgggccttggaaaacagggatgaagcacattcctaa

SEQ ID NO: 4 - protein, 585 aa, Homo sapiens
MTITYTSQVANARLGSFSRLLLCWRGSIYKLLYGEFLIFLLCYYIIRFIYRLALTEEQQLMFEKLTLYCDSYIQLIPISFVLGFYVTLVVTRWWNQYENLPWPDRLMSLVSGFVEGKDEQGRLLRRTLIRYANLGNVLILRSVSTAVYKRFPSAQHLVQAGFMTPAEHKQLEKLSLPHNMFWVPWVWFANLSMKAWLGGRIRDPILLQSLLNEMNTLRTQCGHLYAYDWISIPLVYTQVVTVAVYSFFLTCLVGRQFLNPAKAYPGHELDLVVPVFTFLQFFFYVGWLKVAEQLINPFGEDDDDFETNWIVDRNLQVSLLAVDEMHQDLPRMEPDMYWNKPEPQPPYTAASAQFRRASFMGSTFNISLNKEEMEFQPNQEDEEDAHAGIIGRFLGLQSHDHHPPRANSRTKLLWPKRESLLHEGLPKNHKAAKQNVRGQEDNKAWKLKAVDAFKSAPLYQRPGYYSAPQTPLSPTPMFFPLEPSAPSKLHSVTGIDTKDKSLKTVSSGAKKSFELLSESDGALMEHPEVSQVRRKTVEFNLTDMPEIPENHLKEPLEQSPTNIHTTLKDHMDPYWALENRDEAHS

SEQ ID NO: 5 - skipped

SEQ ID NO: 6 - DNA, 53 bp, Oryctolagus cuniculus
ctcctgggcaacgtgctggttattgtgctgtctcatcattttggcaaagaatt

SEQ ID NO: 7 - DNA, 123 bp, Artificial Sequence
  Recombinant synthesis
gtgccgcagggggacggctgccttcgggggggacggggcagggcggggttcggcttctggcgtgtgaccggcggctctagagcctctgctaaccatgttcatgccttcttctttttcctacag

SEQ ID NO: 8 - DNA, 593 bp, Artificial Sequence
  Recombinant synthesis
atcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctcccc

SEQ ID NO: 9 - DNA, 269 bp, Artificial Sequence
  Recombinant synthesis
cgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctgggg

SEQ ID NO: 10 - DNA, 144 bp, Artificial Sequence
  Recombinant synthesis
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgatt

SEQ ID NO: 11 - DNA, 145 bp, Artificial Sequence
  Recombinant synthesis
tgcgcgctcgctcgctcactgaggccgggcgaccaaaggtcgcccgacgcccgggctttgcccgggcggcctcagtgagcgagcgagcgcgcagagctttttgcaaaagcctaggcctccaaaaaagcctcctcactacttctgg

SEQ ID NO: 12 - DNA, 2500 bp, Artificial Sequence
  Recombinant synthesis
gatgtaaccatatacttaggctggatcttctcccgcgaattttaaccctcaccaactacgagatatgaggtaagccaaaaaagcacgtagtggcgctctccgactgttcccaaattgtaacttatcgttccgtgaaggccagagttacttcccggccctttccatgcgcgcaccataccctcctagttccccggttatctttccgaagtgggagtgagcgaacctccgtttacgtcttgttaccaatgatgtagctatgcactttgtacagggtgccaacgggtttcacaattcacagatagtggggatcccggcaaagggcctatatttgcggtccaacttaggcgtaaacctcgatgctacctactcagacccacctcgcgcggggtaaataaggcactcatcccagctggttcttggcgttctacgcagcgacatgtttattaacagttgtctggcagcacaaaacttttaccatggtcgtagaagccccccagagttagttcatacctaatgccacaaatgtgacaggacgccgatgggtaccggactttaggtcgagcacagttcggtaacggagagaccctgcggcgtacttcattatgtatatggaacgtgcccaagtgacgccaggcaagtctcagctggttcctgtgttagctcgagggtagacatacgagctgattgaacatgggttgggggcctcgaaccgtcgaggaccccatagtacctcggagaccaagtagggcagcctatagtttgaagcagaactatttcggggggcgagccctcatcgtctcttctgcggatgactcaacacgctagggacgtgaagtcgattccttcgatggttataaatcaaagactcagagtgctgtctggagcgtgaatctaacggtacgtatctcgattgctcggtcgcttttcgcactccgcgaaagttcgtaccgctcattcactaggttgcgaagcctatgctgatatatgaatccaaactagagcagggctcttaagattcggagttgtaaatacttaatactccaatcggcttttacgtgcaccaccgcgggcggctgacaagggtctcacatcgagaaacaagacagttccgggctggaagtagcgccggctaaggaagacgcctggtacggcaggactatgaaaccagtacaaaggcaacatcctcacttgggtgaacggaaacgcagtattatggttactttttggatacgtgaaacatatcccatggtagtccttagacttgggagtctatcacccctagggcccatatctggaaatagacgccaggttgaatccgtatttggaggtacgatggaacagtctgggtgggacgtgcttcatttataccctgcgcaggctggaccgaggaccgcaaggtgcggcggtgcacaagcaattgacaactaaccaccgtgtattcattatggtaccaggaactttaagccgagtcaatgaagctcgcattacagtgtttaccgcatcttgccgttactcacaaactgtgatccaccacaagtcaagccattgcctctctgacacgccgtaagaattaatatgtaaactttgcgcgggttgactgcgatccgttcagtctcgtccgagggcacaatcctattcccatttgtatgttcagctaacttctacccatcccccgaagttaagtaggtcgtgagatgccatggaggctctcgttcatcccgtgggacatcaagcttccccttgataaagcaccccgctcgggtgtagcagagaagacgccttctgaattgtgcaatccctccaccttatctaagcttgctaccaataattagcatttttgccttgcgacagacctcctacttagattgccacacattgagctagtcagtgagcgataagcttgacgcgctttcaagggtcgcgagtacgtgaactaaggctccggacaggactatatacttgggtttgatctcgccccgacaactgcaaacctcaacttttttagattatatggttagccgaagttgcacgaggtggcgtccgcggactgctccccgagtgtggctctttcatctgacaacgtgcaacccctatcgcggccgattgtttctgcggacgatgttgtcctcatagtttgggcatgtttcccttgtaggtgtgaaaccacttagcttcgcgccgtagtcccaatgaaaaacctatggactttgttttgggtagcaccaggaatctgaaccgtgtgaatgtggacgtcgcgcgcgtagacctttatctccggttcaagctagggatgtggctgcatgctacgttgtcacacctacactgctcgaagtaaatatgcgaagcgcgcggcctggccggaggcgttccgcgccgccacgtgttcgttaactgttgattggtggcacataagcaatatcgtagtccgtcaaattcagctctgttatcccgggcgttatgtgtcaaatggcgtagaacgggattgactgtttgacggtag

SEQ ID NO: 13 - DNA, 8938 bp, Artificial Sequence
  Recombinant synthesis
tagctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggtaaattctgtcattttactagggtgatgaaattcccaagcaacaccatccttttcagataagggcactgaggctgagagaggagctgaaacctacccggggtcaccacacacaggtggcaaggctgggaccagaaaccaggactgttgactgcagcccggtattcattctttccatagcccacagggctgtcaaagaccccagggcctagtcagaggctcctccttcctggagagttcctggcacagaagttgaagctcagcacagccccctaacccccaactctctctgcaaggcctcaggggtcagaacactggtggagcagatcctttagcctctggattttagggccatggtagagggggtgttgccctaaattccagccctggtctcagcccaacaccctccaagaagaaattagaggggccatggccaggctgtgctagccgttgcttctgagcagattacaagaagggactaagacaaggactcctttgtggaggtcctggcttagggagtcaagtgacggcggctcagcactcacgtgggcagtgccagcctctaagagtgggcaggggcactggccacagagtcccagggagtcccaccagcctagtcgccagaccggcaccatgaccatcacttacacaagccaagtggctaatgcccgcttaggctccttctcccgcctgctgctgtgctggcggggcagcatctacaagctgctatatggcgagttcttaatcttcctgctctgctactacatcatccgctttatttataggctggccctcacggaagaacaacagctgatgtttgagaaactgactctgtattgcgacagctacatccagctcatccccatttccttcgtgctgggcttctacgtgacgctggtcgtgacccgctggtggaaccagtacgagaacctgccgtggcccgaccgcctcatgagcctggtgtcgggcttcgtcgaaggcaaggacgagcaaggccggctgctgcggcgcacgctcatccgctacgccaacctgggcaacgtgctcatcctgcgcagcgtcagcaccgcagtctacaagcgcttccccagcgcccagcacctggtgcaagcaggctttatgactccggcagaacacaagcagttggagaaactgagcctaccacacaacatgttctgggtgccctgggtgtggtttgccaacctgtcaatgaaggcgtggcttggaggtcgaatccgggaccctatcctgctccagagcctgctgaacgagatgaacaccttgcgtactcagtgtggacacctgtatgcctacgactggattagtatcccactggtgtatacacaggtggtgactgtggcggtgtacagcttcttcctgacttgtctagttgggcggcagtttctgaacccagccaaggcctaccctggccatgagctggacctcgttgtgcccgtcttcacgttcctgcagttcttcttctatgttggctggctgaaggtggcagagcagctcatcaacccctttggagaggatgatgatgattttgagaccaactggattgtcgacaggaatttgcaggtgtccctgttggctgtggatgagatgcaccaggacctgcctcggatggagccggacatgtactggaataagcccgagccacagcccccctacacagctgcttccgcccagttccgtcgagcctcctttatgggctccaccttcaacatcagcctgaacaaagaggagatggagttccagcccaatcaggaggacgaggaggatgctcacgctggcatcattggccgcttcctaggcctgcagtcccatgatcaccatcctcccagggcaaactcaaggaccaaactactgtggcccaagagggaatcccttctccacgagggcctgcccaaaaaccacaaggcagccaaacagaacgttaggggccaggaagacaacaaggcctggaagcttaaggctgtggacgccttcaagtctgccccactgtatcagaggccaggctactacagtgccccacagacgcccctcagccccactcccatgttcttccccctagaaccatcagcgccgtcaaagcttcacagtgtcacaggcatagacaccaaagacaaaagcttaaagactgtgagttctggggccaagaaaagttttgaattgctctcagagagcgatggggccttgatggagcacccagaagtatctcaagtgaggaggaaaactgtggagtttaacctgacggatatgccagagatccccgaaaatcacctcaaagaacctttggaacaatcaccaaccaacatacacactacactcaaagatcacatggatccttattgggccttggaaaacagggatgaagcacattcctaatctagcggccgcgaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccggcggccgcgcaccgtcgactcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctcgactagagcatggctacgtagataagtagcatggcgggttaatcattaactacaaggaacccctagtgatggagttggccactccctctctgcgcgctcgctcgctcactgaggccgggcgaccaaaggtcgcccgacgcccgggcggcctcagtgagcgagcgagcgcgcagagctttttgcaaaagcctaggcctccaaaaaagcctcctcactacttctggaatagctcagaggccgaggcggcctcggcctctgcataaataaaaaaaattagtcagccatggggcggagaatgggcggaactgggcggagttaggggcgggatgggcggagttaggggcgggactatggttgctgactaattgagatgcatgctttgcatacttctgcctgctggggagcctggggactttccacacctggttgctgactaattgagatgcatgctttgcatacttctgcctgctggggagcctggggactttccacaccctaactgacacacattccacagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcctgcaaaccacgttgtgtctcaaaatctctgatgttacattgcacaagataaaaatatatcatcatgaacaataaaactgtctgcttacataaacagtaatacaaggggtgttatgagccatattcaacgggaaacgtcttgctcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaagggcggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatctggctagcgatgaccctgctgattggttcgctgaccatttccgggtgcgggacggcgttaccagaaactcagaaggttcgtccaaccaaaccgactctgacggcagtttacgagagagatgatagggtctgcttcagggtgaccgatgtaaccatatacttaggctggatcttctcccgcgaattttaaccctcaccaactacgagatatgaggtaagccaaaaaagcacgtagtggcgctctccgactgttcccaaattgtaacttatcgttccgtgaaggccagagttacttcccggccctttccatgcgcgcaccataccctcctagttccccggttatctttccgaagtgggagtgagcgaacctccgtttacgtcttgttaccaatgatgtagctatgcactttgtacagggtgccaacgggtttcacaattcacagatagtggggatcccggcaaagggcctatatttgcggtccaacttaggcgtaaacctcgatgctacctactcagacccacctcgcgcggggtaaataaggcactcatcccagctggttcttggcgttctacgcagcgacatgtttattaacagttgtctggcagcacaaaacttttaccatggtcgtagaagccccccagagttagttcatacctaatgccacaaatgtgacaggacgccgatgggtaccggactttaggtcgagcacagttcggtaacggagagaccctgcggcgtacttcattatgtatatggaacgtgcccaagtgacgccaggcaagtctcagctggttcctgtgttagctcgagggtagacatacgagctgattgaacatgggttgggggcctcgaaccgtcgaggaccccatagtacctcggagaccaagtagggcagcctatagtttgaagcagaactatttcggggggcgagccctcatcgtctcttctgcggatgactcaacacgctagggacgtgaagtcgattccttcgatggttataaatcaaagactcagagtgctgtctggagcgtgaatctaacggtacgtatctcgattgctcggtcgcttttcgcactccgcgaaagttcgtaccgctcattcactaggttgcgaagcctatgctgatatatgaatccaaactagagcagggctcttaagattcggagttgtaaatacttaatactccaatcggcttttacgtgcaccaccgcgggcggctgacaagggtctcacatcgagaaacaagacagttccgggctggaagtagcgccggctaaggaagacgcctggtacggcaggactatgaaaccagtacaaaggcaacatcctcacttgggtgaacggaaacgcagtattatggttactttttggatacgtgaaacatatcccatggtagtccttagacttgggagtctatcacccctagggcccatatctggaaatagacgccaggttgaatccgtatttggaggtacgatggaacagtctgggtgggacgtgcttcatttataccctgcgcaggctggaccgaggaccgcaaggtgcggcggtgcacaagcaattgacaactaaccaccgtgtattcattatggtaccaggaactttaagccgagtcaatgaagctcgcattacagtgtttaccgcatcttgccgttactcacaaactgtgatccaccacaagtcaagccattgcctctctgacacgccgtaagaattaatatgtaaactttgcgcgggttgactgcgatccgttcagtctcgtccgagggcacaatcctattcccatttgtatgttcagctaacttctacccatcccccgaagttaagtaggtcgtgagatgccatggaggctctcgttcatcccgtgggacatcaagcttccccttgataaagcaccccgctcgggtgtagcagagaagacgccttctgaattgtgcaatccctccaccttatctaagcttgctaccaataattagcatttttgccttgcgacagacctcctacttagattgccacacattgagctagtcagtgagcgataagcttgacgcgctttcaagggtcgcgagtacgtgaactaaggctccggacaggactatatacttgggtttgatctcgccccgacaactgcaaacctcaacttttttagattatatggttagccgaagttgcacgaggtggcgtccgcggactgctccccgagtgtggctctttcatctgacaacgtgcaacccctatcgcggccgattgtttctgcggacgatgttgtcctcatagtttgggcatgtttcccttgtaggtgtgaaaccacttagcttcgcgccgtagtcccaatgaaaaacctatggactttgttttgggtagcaccaggaatctgaaccgtgtgaatgtggacgtcgcgcgcgtagacctttatctccggttcaagctagggatgtggctgcatgctacgttgtcacacctacactgctcgaagtaaatatgcgaagcgcgcggcctggccggaggcgttccgcgccgccacgtgttcgttaactgttgattggtggcacataagcaatatcgtagtccgtcaaattcagctctgttatcccgggcgttatgtgtcaaatggcgtagaacgggattgactgtttgacggtagggtgacctaagccagatgctacacaattaggcttgtacatattgtcgttagaacgcggctacaattaatacataaccttatgtatcatacacatacgatttaggtgacactatagaatacacggaattaattc

SEQ ID NO: 14 - DNA, 9116 bp, Artificial Sequence
  Recombinant synthesis
tagctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggtaaattctgtcattttactagggtgatgaaattcccaagcaacaccatccttttcagataagggcactgaggctgagagaggagctgaaacctacccggggtcaccacacacaggtggcaaggctgggaccagaaaccaggactgttgactgcagcccggtattcattctttccatagcccacagggctgtcaaagaccccagggcctagtcagaggctcctccttcctggagagttcctggcacagaagttgaagctcagcacagccccctaacccccaactctctctgcaaggcctcaggggtcagaacactggtggagcagatcctttagcctctggattttagggccatggtagagggggtgttgccctaaattccagccctggtctcagcccaacaccctccaagaagaaattagaggggccatggccaggctgtgctagccgttgcttctgagcagattacaagaagggactaagacaaggactcctttgtggaggtcctggcttagggagtcaagtgacggcggctcagcactcacgtgggcagtgccagcctctaagagtgggcaggggcactggccacagagtcccagggagtcccaccagcctagtcgccagaccgggtgccgcagggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattggcaccatgaccatcacttacacaagccaagtggctaatgcccgcttaggctccttctcccgcctgctgctgtgctggcggggcagcatctacaagctgctatatggcgagttcttaatcttcctgctctgctactacatcatccgctttatttataggctggccctcacggaagaacaacagctgatgtttgagaaactgactctgtattgcgacagctacatccagctcatccccatttccttcgtgctgggcttctacgtgacgctggtcgtgacccgctggtggaaccagtacgagaacctgccgtggcccgaccgcctcatgagcctggtgtcgggcttcgtcgaaggcaaggacgagcaaggccggctgctgcggcgcacgctcatccgctacgccaacctgggcaacgtgctcatcctgcgcagcgtcagcaccgcagtctacaagcgcttccccagcgcccagcacctggtgcaagcaggctttatgactccggcagaacacaagcagttggagaaactgagcctaccacacaacatgttctgggtgccctgggtgtggtttgccaacctgtcaatgaaggcgtggcttggaggtcgaatccgggaccctatcctgctccagagcctgctgaacgagatgaacaccttgcgtactcagtgtggacacctgtatgcctacgactggattagtatcccactggtgtatacacaggtggtgactgtggcggtgtacagcttcttcctgacttgtctagttgggcggcagtttctgaacccagccaaggcctaccctggccatgagctggacctcgttgtgcccgtcttcacgttcctgcagttcttcttctatgttggctggctgaaggtggcagagcagctcatcaacccctttggagaggatgatgatgattttgagaccaactggattgtcgacaggaatttgcaggtgtccctgttggctgtggatgagatgcaccaggacctgcctcggatggagccggacatgtactggaataagcccgagccacagcccccctacacagctgcttccgcccagttccgtcgagcctcctttatgggctccaccttcaacatcagcctgaacaaagaggagatggagttccagcccaatcaggaggacgaggaggatgctcacgctggcatcattggccgcttcctaggcctgcagtcccatgatcaccatcctcccagggcaaactcaaggaccaaactactgtggcccaagagggaatcccttctccacgagggcctgcccaaaaaccacaaggcagccaaacagaacgttaggggccaggaagacaacaaggcctggaagcttaaggctgtggacgccttcaagtctgccccactgtatcagaggccaggctactacagtgccccacagacgcccctcagccccactcccatgttcttccccctagaaccatcagcgccgtcaaagcttcacagtgtcacaggcatagacaccaaagacaaaagcttaaagactgtgagttctggggccaagaaaagttttgaattgctctcagagagcgatggggccttgatggagcacccagaagtatctcaagtgaggaggaaaactgtggagtttaacctgacggatatgccagagatccccgaaaatcacctcaaagaacctttggaacaatcaccaaccaacatacacactacactcaaagatcacatggatccttattgggccttggaaaacagggatgaagcacattcctaatctagcggccgcgaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccggcggccgcgcaccgtcgactcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctcgactagagcatggctacgtagataagtagcatggcgggttaatcattaactacaaggaacccctagtgatggagttggccactccctctctgcgcgctcgctcgctcactgaggccgggcgaccaaaggtcgcccgacgcccgggcggcctcagtgagcgagcgagcgcgcagagctttttgcaaaagcctaggcctccaaaaaagcctcctcactacttctggaatagctcagaggccgaggcggcctcggcctctgcataaataaaaaaaattagtcagccatggggcggagaatgggcggaactgggcggagttaggggcgggatgggcggagttaggggcgggactatggttgctgactaattgagatgcatgctttgcatacttctgcctgctggggagcctggggactttccacacctggttgctgactaattgagatgcatgctttgcatacttctgcctgctggggagcctggggactttccacaccctaactgacacacattccacagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcctgcaaaccacgttgtgtctcaaaatctctgatgttacattgcacaagataaaaatatatcatcatgaacaataaaactgtctgcttacataaacagtaatacaaggggtgttatgagccatattcaacgggaaacgtcttgctcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaagggcggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatctggctagcgatgaccctgctgattggttcgctgaccatttccgggtgcgggacggcgttaccagaaactcagaaggttcgtccaaccaaaccgactctgacggcagtttacgagagagatgatagggtctgcttcagggtgaccgatgtaaccatatacttaggctggatcttctcccgcgaattttaaccctcaccaactacgagatatgaggtaagccaaaaaagcacgtagtggcgctctccgactgttcccaaattgtaacttatcgttccgtgaaggccagagttacttcccggccctttccatgcgcgcaccataccctcctagttccccggttatctttccgaagtgggagtgagcgaacctccgtttacgtcttgttaccaatgatgtagctatgcactttgtacagggtgccaacgggtttcacaattcacagatagtggggatcccggcaaagggcctatatttgcggtccaacttaggcgtaaacctcgatgctacctactcagacccacctcgcgcggggtaaataaggcactcatcccagctggttcttggcgttctacgcagcgacatgtttattaacagttgtctggcagcacaaaacttttaccatggtcgtagaagccccccagagttagttcatacctaatgccacaaatgtgacaggacgccgatgggtaccggactttaggtcgagcacagttcggtaacggagagaccctgcggcgtacttcattatgtatatggaacgtgcccaagtgacgccaggcaagtctcagctggttcctgtgttagctcgagggtagacatacgagctgattgaacatgggttgggggcctcgaaccgtcgaggaccccatagtacctcggagaccaagtagggcagcctatagtttgaagcagaactatttcggggggcgagccctcatcgtctcttctgcggatgactcaacacgctagggacgtgaagtcgattccttcgatggttataaatcaaagactcagagtgctgtctggagcgtgaatctaacggtacgtatctcgattgctcggtcgcttttcgcactccgcgaaagttcgtaccgctcattcactaggttgcgaagcctatgctgatatatgaatccaaactagagcagggctcttaagattcggagttgtaaatacttaatactccaatcggcttttacgtgcaccaccgcgggcggctgacaagggtctcacatcgagaaacaagacagttccgggctggaagtagcgccggctaaggaagacgcctggtacggcaggactatgaaaccagtacaaaggcaacatcctcacttgggtgaacggaaacgcagtattatggttactttttggatacgtgaaacatatcccatggtagtccttagacttgggagtctatcacccctagggcccatatctggaaatagacgccaggttgaatccgtatttggaggtacgatggaacagtctgggtgggacgtgcttcatttataccctgcgcaggctggaccgaggaccgcaaggtgcggcggtgcacaagcaattgacaactaaccaccgtgtattcattatggtaccaggaactttaagccgagtcaatgaagctcgcattacagtgtttaccgcatcttgccgttactcacaaactgtgatccaccacaagtcaagccattgcctctctgacacgccgtaagaattaatatgtaaactttgcgcgggttgactgcgatccgttcagtctcgtccgagggcacaatcctattcccatttgtatgttcagctaacttctacccatcccccgaagttaagtaggtcgtgagatgccatggaggctctcgttcatcccgtgggacatcaagcttccccttgataaagcaccccgctcgggtgtagcagagaagacgccttctgaattgtgcaatccctccaccttatctaagcttgctaccaataattagcatttttgccttgcgacagacctcctacttagattgccacacattgagctagtcagtgagcgataagcttgacgcgctttcaagggtcgcgagtacgtgaactaaggctccggacaggactatatacttgggtttgatctcgccccgacaactgcaaacctcaacttttttagattatatggttagccgaagttgcacgaggtggcgtccgcggactgctccccgagtgtggctctttcatctgacaacgtgcaacccctatcgcggccgattgtttctgcggacgatgttgtcctcatagtttgggcatgtttcccttgtaggtgtgaaaccacttagcttcgcgccgtagtcccaatgaaaaacctatggactttgttttgggtagcaccaggaatctgaaccgtgtgaatgtggacgtcgcgcgcgtagacctttatctccggttcaagctagggatgtggctgcatgctacgttgtcacacctacactgctcgaagtaaatatgcgaagcgcgcggcctggccggaggcgttccgcgccgccacgtgttcgttaactgttgattggtggcacataagcaatatcgtagtccgtcaaattcagctctgttatcccgggcgttatgtgtcaaatggcgtagaacgggattgactgtttgacggtagggtgacctaagccagatgctacacaattaggcttgtacatattgtcgttagaacgcggctacaattaatacataaccttatgtatcatacacatacgatttaggtgacactatagaatacacggaattaattc

SEQ ID NO: 15 - DNA, 7109 bp, Artificial Sequence
  Recombinant synthesis
tagctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggtaccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattggatccgcggccgcagcttggtaccgccaccatgaccatcacttacacaagccaagtggctaatgcccgcttaggctccttctcccgcctgctgctgtgctggcggggcagcatctacaagctgctatatggcgagttcttaatcttcctgctctgctactacatcatccgctttatttataggctggccctcacggaagaacaacagctgatgtttgagaaactgactctgtattgcgacagttacatccagctcatccccatttccttcgtgctgggcttctacgtgacgctggtcgtgacccgctggtggaaccagtacgagaacctgccgtggcccgaccgcctcatgagcctggtgtcgggcttcgtcgaaggcaaggacgagcaaggccggctgctgcggcgcacgctcatccgctacgccaacctgggcaacgtgctcatcctgcgcagcgtcagcaccgcagtctacaagcgcttccccagcgcccagcacctggtgcaagcaggctttatgactccggcagaacacaagcagttggagaaactgagcctaccacacaacatgttctgggtgccctgggtgtggtttgccaacctgtcaatgaaggcgtggcttggaggtcgaatccgggaccctatcctgctccagagcctgctgaacgagatgaacaccttgcgtactcagtgtggacacctgtatgcctacgactggattagtatcccactggtgtatacacaggtggtgactgtggcggtgtacagcttcttcctgacttgtctagttgggcggcagtttctgaacccagccaaggcctaccctggccatgagctggacctcgttgtgcccgtcttcacgttcctgcagttcttcttctatgttggctggctgaaggtggcagagcagctcatcaacccctttggagaggatgatgatgattttgagaccaactggattgtcgacaggaatttgcaggtgtccctgttggctgtggatgagatgcaccaggacctgcctcggatggagccggacatgtactggaataagcccgagccacagcccccctacacagctgcttccgcccagttccgtcgagcctcctttatgggctccaccttcaacatcagcctgaacaaagaggagatggagttccagcccaatcaggaggacgaggaggatgctcacgctggcatcattggccgcttcctaggcctgcagtcccatgatcaccatcctcccagggcaaactcaaggaccaaactactgtggcccaagagggaatcccttctccacgagggcctgcccaaaaaccacaaggcagccaaacagaacgttaggggccaggaagacaacaaggcctggaagcttaaggctgtggacgccttcaagtctgccccactgtatcagaggccaggctactacagtgccccacagacgcccctcagccccactcccatgttcttccccctagaaccatcagcgccgtcaaagcttcacagtgtcacaggcatagacaccaaagacaaaagcttaaagactgtgagttctggggccaagaaaagttttgaattgctctcagagagcgatggggccttgatggagcacccagaagtatctcaagtgaggaggaaaactgtggagtttaacctgacggatatgccagagatccccgaaaatcacctcaaagaacctttggaacaatcaccaaccaacatacacactacactcaaagatcacatggatccttattgggccttggaaaacagggatgaagcacattcctaagagctcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgactcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctcgactagagcatggctacgtagataagtagcatggcgggttaatcattaactacaaggaacccctagtgatggagttggccactccctctctgcgcgctcgctcgctcactgaggccgggcgaccaaaggtcgcccgacgcccgggcggcctcagtgagcgagcgagcgcgcagagctttttgcaaaagcctaggcctccaaaaaagcctcctcactacttctggaatagctcagaggccgaggcggcctcggcctctgcataaataaaaaaaattagtcagccatggggcggagaatgggcggaactgggcggagttaggggcgggatgggcggagttaggggcgggactatggttgctgactaattgagatgcatgctttgcatacttctgcctgctggggagcctggggactttccacacctggttgctgactaattgagatgcatgctttgcatacttctgcctgctggggagcctggggactttccacaccctaactgacacacattccacagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccattcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatctggctagcgatgaccctgctgattggttcgctgaccatttccgggtgcgggacggcgttaccagaaactcagaaggttcgtccaaccaaaccgactctgacggcagtttacgagagagatgatagggtctgcttcagtaagccagatgctacacaattaggcttgtacatattgtcgttagaacgcggctacaattaatacataaccttatgtatcatacacatacgatttaggtgacactatagaatacacggaattaattc

SEQ ID NO: 16 - DNA, 7873 bp, Artificial Sequence
  Recombinant synthesis
tagctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggtaaattctgtcattttactagggtgatgaaattcccaagcaacaccatccttttcagataagggcactgaggctgagagaggagctgaaacctacccggggtcaccacacacaggtggcaaggctgggaccagaaaccaggactgttgactgcagcccggtattcattctttccatagcccacagggctgtcaaagaccccagggcctagtcagaggctcctccttcctggagagttcctggcacagaagttgaagctcagcacagccccctaacccccaactctctctgcaaggcctcaggggtcagaacactggtggagcagatcctttagcctctggattttagggccatggtagagggggtgttgccctaaattccagccctggtctcagcccaacaccctccaagaagaaattagaggggccatggccaggctgtgctagccgttgcttctgagcagattacaagaagggactaagacaaggactcctttgtggaggtcctggcttagggagtcaagtgacggcggctcagcactcacgtgggcagtgccagcctctaagagtgggcaggggcactggccacagagtcccagggagtcccaccagcctagtcgccagaccggcaccatgagcaagggcgaggaactgttcactggcgtggtcccaattctcgtggaactggatggcgatgtgaatgggcacaaattttctgtcagcggagagggtgaaggtgatgccacatacggaaagctcaccctgaaattcatctgcaccactggaaagctccctgtgccatggccaacactggtcactaccctgacctatggcgtgcagtgcttttccagatacccagaccatatgaagcagcatgactttttcaagagcgccatgcccgagggctatgtgcaggagagaaccatctttttcaaagatgacgggaactacaagacccgcgctgaagtcaagttcgaaggtgacaccctggtgaatagaatcgagctgaagggcattgactttaaggaggatggaaacattctcggccacaagctggaatacaactataactcccacaatgtgtacatcatggccgacaagcaaaagaatggcatcaaggtcaacttcaagatcagacacaacattgaggatggatccgtgcagctggccgaccattatcaacagaacactccaatcggcgacggccctgtgctcctcccagacaaccattacctgtccacccagtctgccctgtctaaagatcccaacgaaaagagagaccacatggtcctgctggagtttgtgaccgctgctgggatcacacatggcatggacgagctgtacaagtgaaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccggcggccgcgcaccgtcgactcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctcgactagagcatggctacgtagataagtagcatggcgggttaatcattaactacaaggaacccctagtgatggagttggccactccctctctgcgcgctcgctcgctcactgaggccgggcgaccaaaggtcgcccgacgcccgggcggcctcagtgagcgagcgagcgcgcagagctttttgcaaaagcctaggcctccaaaaaagcctcctcactacttctggaatagctcagaggccgaggcggcctcggcctctgcataaataaaaaaaattagtcagccatggggcggagaatgggcggaactgggcggagttaggggcgggatgggcggagttaggggcgggactatggttgctgactaattgagatgcatgctttgcatacttctgcctgctggggagcctggggactttccacacctggttgctgactaattgagatgcatgctttgcatacttctgcctgctggggagcctggggactttccacaccctaactgacacacattccacagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcctgcaaaccacgttgtgtctcaaaatctctgatgttacattgcacaagataaaaatatatcatcatgaacaataaaactgtctgcttacataaacagtaatacaaggggtgttatgagccatattcaacgggaaacgtcttgctcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaagggcggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatctggctagcgatgaccctgctgattggttcgctgaccatttccgggtgcgggacggcgttaccagaaactcagaaggttcgtccaaccaaaccgactctgacggcagtttacgagagagatgatagggtctgcttcagggtgaccgatgtaaccatatacttaggctggatcttctcccgcgaattttaaccctcaccaactacgagatatgaggtaagccaaaaaagcacgtagtggcgctctccgactgttcccaaattgtaacttatcgttccgtgaaggccagagttacttcccggccctttccatgcgcgcaccataccctcctagttccccggttatctttccgaagtgggagtgagcgaacctccgtttacgtcttgttaccaatgatgtagctatgcactttgtacagggtgccaacgggtttcacaattcacagatagtggggatcccggcaaagggcctatatttgcggtccaacttaggcgtaaacctcgatgctacctactcagacccacctcgcgcggggtaaataaggcactcatcccagctggttcttggcgttctacgcagcgacatgtttattaacagttgtctggcagcacaaaacttttaccatggtcgtagaagccccccagagttagttcatacctaatgccacaaatgtgacaggacgccgatgggtaccggactttaggtcgagcacagttcggtaacggagagaccctgcggcgtacttcattatgtatatggaacgtgcccaagtgacgccaggcaagtctcagctggttcctgtgttagctcgagggtagacatacgagctgattgaacatgggttgggggcctcgaaccgtcgaggaccccatagtacctcggagaccaagtagggcagcctatagtttgaagcagaactatttcggggggcgagccctcatcgtctcttctgcggatgactcaacacgctagggacgtgaagtcgattccttcgatggttataaatcaaagactcagagtgctgtctggagcgtgaatctaacggtacgtatctcgattgctcggtcgcttttcgcactccgcgaaagttcgtaccgctcattcactaggttgcgaagcctatgctgatatatgaatccaaactagagcagggctcttaagattcggagttgtaaatacttaatactccaatcggcttttacgtgcaccaccgcgggcggctgacaagggtctcacatcgagaaacaagacagttccgggctggaagtagcgccggctaaggaagacgcctggtacggcaggactatgaaaccagtacaaaggcaacatcctcacttgggtgaacggaaacgcagtattatggttactttttggatacgtgaaacatatcccatggtagtccttagacttgggagtctatcacccctagggcccatatctggaaatagacgccaggttgaatccgtatttggaggtacgatggaacagtctgggtgggacgtgcttcatttataccctgcgcaggctggaccgaggaccgcaaggtgcggcggtgcacaagcaattgacaactaaccaccgtgtattcattatggtaccaggaactttaagccgagtcaatgaagctcgcattacagtgtttaccgcatcttgccgttactcacaaactgtgatccaccacaagtcaagccattgcctctctgacacgccgtaagaattaatatgtaaactttgcgcgggttgactgcgatccgttcagtctcgtccgagggcacaatcctattcccatttgtatgttcagctaacttctacccatcccccgaagttaagtaggtcgtgagatgccatggaggctctcgttcatcccgtgggacatcaagcttccccttgataaagcaccccgctcgggtgtagcagagaagacgccttctgaattgtgcaatccctccaccttatctaagcttgctaccaataattagcatttttgccttgcgacagacctcctacttagattgccacacattgagctagtcagtgagcgataagcttgacgcgctttcaagggtcgcgagtacgtgaactaaggctccggacaggactatatacttgggtttgatctcgccccgacaactgcaaacctcaacttttttagattatatggttagccgaagttgcacgaggtggcgtccgcggactgctccccgagtgtggctctttcatctgacaacgtgcaacccctatcgcggccgattgtttctgcggacgatgttgtcctcatagtttgggcatgtttcccttgtaggtgtgaaaccacttagcttcgcgccgtagtcccaatgaaaaacctatggactttgttttgggtagcaccaggaatctgaaccgtgtgaatgtggacgtcgcgcgcgtagacctttatctccggttcaagctagggatgtggctgcatgctacgttgtcacacctacactgctcgaagtaaatatgcgaagcgcgcggcctggccggaggcgttccgcgccgccacgtgttcgttaactgttgattggtggcacataagcaatatcgtagtccgtcaaattcagctctgttatcccgggcgttatgtgtcaaatggcgtagaacgggattgactgtttgacggtagggtgacctaagccagatgctacacaattaggcttgtacatattgtcgttagaacgcggctacaattaatacataaccttatgtatcatacacatacgatttaggtgacactatagaatacacggaattaattc

SEQ ID NO: 17 - DNA, 8051 bp, Artificial Sequence
  Recombinant synthesis
tagctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggtaaattctgtcattttactagggtgatgaaattcccaagcaacaccatccttttcagataagggcactgaggctgagagaggagctgaaacctacccggggtcaccacacacaggtggcaaggctgggaccagaaaccaggactgttgactgcagcccggtattcattctttccatagcccacagggctgtcaaagaccccagggcctagtcagaggctcctccttcctggagagttcctggcacagaagttgaagctcagcacagccccctaacccccaactctctctgcaaggcctcaggggtcagaacactggtggagcagatcctttagcctctggattttagggccatggtagagggggtgttgccctaaattccagccctggtctcagcccaacaccctccaagaagaaattagaggggccatggccaggctgtgctagccgttgcttctgagcagattacaagaagggactaagacaaggactcctttgtggaggtcctggcttagggagtcaagtgacggcggctcagcactcacgtgggcagtgccagcctctaagagtgggcaggggcactggccacagagtcccagggagtcccaccagcctagtcgccagaccgggtgccgcagggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattggcaccatgagcaagggcgaggaactgttcactggcgtggtcccaattctcgtggaactggatggcgatgtgaatgggcacaaattttctgtcagcggagagggtgaaggtgatgccacatacggaaagctcaccctgaaattcatctgcaccactggaaagctccctgtgccatggccaacactggtcactaccctgacctatggcgtgcagtgcttttccagatacccagaccatatgaagcagcatgactttttcaagagcgccatgcccgagggctatgtgcaggagagaaccatctttttcaaagatgacgggaactacaagacccgcgctgaagtcaagttcgaaggtgacaccctggtgaatagaatcgagctgaagggcattgactttaaggaggatggaaacattctcggccacaagctggaatacaactataactcccacaatgtgtacatcatggccgacaagcaaaagaatggcatcaaggtcaacttcaagatcagacacaacattgaggatggatccgtgcagctggccgaccattatcaacagaacactccaatcggcgacggccctgtgctcctcccagacaaccattacctgtccacccagtctgccctgtctaaagatcccaacgaaaagagagaccacatggtcctgctggagtttgtgaccgctgctgggatcacacatggcatggacgagctgtacaagtgaaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccggcggccgcgcaccgtcgactcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctcgactagagcatggctacgtagataagtagcatggcgggttaatcattaactacaaggaacccctagtgatggagttggccactccctctctgcgcgctcgctcgctcactgaggccgggcgaccaaaggtcgcccgacgcccgggcggcctcagtgagcgagcgagcgcgcagagctttttgcaaaagcctaggcctccaaaaaagcctcctcactacttctggaatagctcagaggccgaggcggcctcggcctctgcataaataaaaaaaattagtcagccatggggcggagaatgggcggaactgggcggagttaggggcgggatgggcggagttaggggcgggactatggttgctgactaattgagatgcatgctttgcatacttctgcctgctggggagcctggggactttccacacctggttgctgactaattgagatgcatgctttgcatacttctgcctgctggggagcctggggactttccacaccctaactgacacacattccacagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcctgcaaaccacgttgtgtctcaaaatctctgatgttacattgcacaagataaaaatatatcatcatgaacaataaaactgtctgcttacataaacagtaatacaaggggtgttatgagccatattcaacgggaaacgtcttgctcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaagggcggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatctggctagcgatgaccctgctgattggttcgctgaccatttccgggtgcgggacggcgttaccagaaactcagaaggttcgtccaaccaaaccgactctgacggcagtttacgagagagatgatagggtctgcttcagggtgaccgatgtaaccatatacttaggctggatcttctcccgcgaattttaaccctcaccaactacgagatatgaggtaagccaaaaaagcacgtagtggcgctctccgactgttcccaaattgtaacttatcgttccgtgaaggccagagttacttcccggccctttccatgcgcgcaccataccctcctagttccccggttatctttccgaagtgggagtgagcgaacctccgtttacgtcttgttaccaatgatgtagctatgcactttgtacagggtgccaacgggtttcacaattcacagatagtggggatcccggcaaagggcctatatttgcggtccaacttaggcgtaaacctcgatgctacctactcagacccacctcgcgcggggtaaataaggcactcatcccagctggttcttggcgttctacgcagcgacatgtttattaacagttgtctggcagcacaaaacttttaccatggtcgtagaagccccccagagttagttcatacctaatgccacaaatgtgacaggacgccgatgggtaccggactttaggtcgagcacagttcggtaacggagagaccctgcggcgtacttcattatgtatatggaacgtgcccaagtgacgccaggcaagtctcagctggttcctgtgttagctcgagggtagacatacgagctgattgaacatgggttgggggcctcgaaccgtcgaggaccccatagtacctcggagaccaagtagggcagcctatagtttgaagcagaactatttcggggggcgagccctcatcgtctcttctgcggatgactcaacacgctagggacgtgaagtcgattccttcgatggttataaatcaaagactcagagtgctgtctggagcgtgaatctaacggtacgtatctcgattgctcggtcgcttttcgcactccgcgaaagttcgtaccgctcattcactaggttgcgaagcctatgctgatatatgaatccaaactagagcagggctcttaagattcggagttgtaaatacttaatactccaatcggcttttacgtgcaccaccgcgggcggctgacaagggtctcacatcgagaaacaagacagttccgggctggaagtagcgccggctaaggaagacgcctggtacggcaggactatgaaaccagtacaaaggcaacatcctcacttgggtgaacggaaacgcagtattatggttactttttggatacgtgaaacatatcccatggtagtccttagacttgggagtctatcacccctagggcccatatctggaaatagacgccaggttgaatccgtatttggaggtacgatggaacagtctgggtgggacgtgcttcatttataccctgcgcaggctggaccgaggaccgcaaggtgcggcggtgcacaagcaattgacaactaaccaccgtgtattcattatggtaccaggaactttaagccgagtcaatgaagctcgcattacagtgtttaccgcatcttgccgttactcacaaactgtgatccaccacaagtcaagccattgcctctctgacacgccgtaagaattaatatgtaaactttgcgcgggttgactgcgatccgttcagtctcgtccgagggcacaatcctattcccatttgtatgttcagctaacttctacccatcccccgaagttaagtaggtcgtgagatgccatggaggctctcgttcatcccgtgggacatcaagcttccccttgataaagcaccccgctcgggtgtagcagagaagacgccttctgaattgtgcaatccctccaccttatctaagcttgctaccaataattagcatttttgccttgcgacagacctcctacttagattgccacacattgagctagtcagtgagcgataagcttgacgcgctttcaagggtcgcgagtacgtgaactaaggctccggacaggactatatacttgggtttgatctcgccccgacaactgcaaacctcaacttttttagattatatggttagccgaagttgcacgaggtggcgtccgcggactgctccccgagtgtggctctttcatctgacaacgtgcaacccctatcgcggccgattgtttctgcggacgatgttgtcctcatagtttgggcatgtttcccttgtaggtgtgaaaccacttagcttcgcgccgtagtcccaatgaaaaacctatggactttgttttgggtagcaccaggaatctgaaccgtgtgaatgtggacgtcgcgcgcgtagacctttatctccggttcaagctagggatgtggctgcatgctacgttgtcacacctacactgctcgaagtaaatatgcgaagcgcgcggcctggccggaggcgttccgcgccgccacgtgttcgttaactgttgattggtggcacataagcaatatcgtagtccgtcaaattcagctctgttatcccgggcgttatgtgtcaaatggcgtagaacgggattgactgtttgacggtagggtgacctaagccagatgctacacaattaggcttgtacatattgtcgttagaacgcggctacaattaatacataaccttatgtatcatacacatacgatttaggtgacactatagaatacacggaattaattc

(19) United States
(12) Patent Application Publication
(10) Pub. No.: US 2019/0307900 A1
(43) Pub. Date: Oct. 10, 2019

(54) COMPOSITIONS AND METHODS FOR TREATING MACULAR DYSTROPHY

(71) Applicants: Oxford University Innovation Limited, Oxford (GB); NightstaRx Limited, London (GB)

(72) Inventors: Robert MACLAREN, Oxford (GB); Christina MARTINEZ-FERNANDEZ DE LA CAMAR, Oxford (GB); Gregory S. ROBINSON, Waltham, MA (US)

(21) Appl. No.: 16/376,808

(22) Filed: Apr. 5, 2019

Related U.S. Application Data
(60) Provisional application No. 62/653,131, filed on Apr. 5, 2018.

(51) Int. Cl. A61K 48/00; A61K 9/00; A61P 27/02

(52) U.S. Cl. CPC A61K 48/0058; A61K 9/0048; A61P 27/02; A61K 48/0091; A61K 9/0019

(57) ABSTRACT

The disclosure provides composition comprising a nucleic acid sequence comprising (a) a sequence encoding a vitelliform macular dystrophy-2 (VMD2) promoter, and (b) a sequence encoding a Bestrophin-1 (BEST1) protein as well as the use of these compositions for the treatment of macular dystrophy in a subject comprising administration of the composition to an eye of a subject via a subretinal or a suprachoroidal route.

### RELATED APPLICATIONS

This application claims the benefit of provisional application U.S. Ser. No. 62/653,131, filed Apr. 5, 2018, the contents of which are herein incorporated by reference in their entirety.

### INCORPORATION OF SEQUENCE LISTING

The contents of the text file named “NIGH-011/002 WO_SeqList.txt,” which was created on Apr. 4, 2019 and is 72 KB in size, are hereby incorporated by reference in their entirety.

### FIELD OF THE DISCLOSURE

The invention relates to the fields of molecular biology, neurobiology and gene therapy treatments for degenerative eye diseases.

### BACKGROUND

Macular degeneration is a medical condition, which may result in blurred or no vision in the center of the visual field. In macular degeneration, the photoreceptors in the part of the retina called the macula, which is responsible for central vision, degenerate or die. In some cases, macular degeneration is caused by mutations in the Bestrophin-1 gene (BEST1, also called VMD2). There is currently no treatment for this devastating disease. There is thus a long felt need in the art for additional therapeutic approaches to treat macular degeneration. The disclosure provides compositions and methods of treatment for macular degeneration.

### SUMMARY

The disclosure provides a composition comprising a nucleic acid sequence comprising: (a) a sequence encoding a vitelliform macular dystrophy-2 (VMD2) promoter, and (b) a sequence encoding a Bestrophin-1 (BEST1) protein. In some embodiments, the sequence encoding the VMD2 promoter encodes a human VMD2 promoter. In some embodiments, the sequence encoding the BEST1 protein encodes a human BEST1 protein. In some embodiments, the sequence encoding the BEST1 protein comprises a coding sequence. In some embodiments, the sequence encoding the BEST1 protein comprises a cDNA sequence.

The disclosure provides a composition comprising a nucleic acid sequence comprising: (a) a sequence encoding a ubiquitous promoter, and (b) a sequence encoding a Bestrophin-1 (BEST1) protein. In some embodiments, the sequence encoding the BEST1 protein encodes a human BEST1 protein. In some embodiments, the sequence encoding the BEST1 protein comprises a coding sequence. In some embodiments, the sequence encoding the BEST1 protein comprises a cDNA sequence. In some embodiments, the sequence encoding a ubiquitous promoter comprises a sequence encoding a CAG promoter.

In some embodiments of the compositions of the disclosure, the nucleic acid sequence further comprises: (c) a sequence encoding a posttranscriptional regulatory element (PRE). In some embodiments, the sequence encoding the PRE comprises a sequence isolated or derived from a naturally occurring sequence. In some embodiments, the sequence encoding the PRE comprises a sequence isolated or derived from a non-naturally-occurring sequence. In some embodiments, the sequence encoding the PRE comprises a sequence isolated or derived from a viral sequence. In some embodiments, the sequence encoding the PRE comprises a sequence isolated or derived from a woodchuck hepatitis virus (WPRE).

In some embodiments of the compositions of the disclosure, the nucleic acid sequence further comprises: (d) a sequence encoding a polyadenylation (polyA) signal. In some embodiments, the sequence encoding the polyA signal comprises a sequence isolated or derived from a naturally occurring sequence. In some embodiments, the sequence encoding the polyA signal comprises a sequence isolated or derived from a non-naturally-occurring sequence. In some embodiments, the sequence encoding the polyA signal comprises a sequence isolated or derived from a mammalian sequence. In some embodiments, the sequence encoding the polyA signal comprises a sequence isolated or derived from a human sequence. In some embodiments, the sequence encoding the polyA signal comprises a sequence isolated or derived from a mammalian Bovine Growth Hormone (BGH) gene.

In some embodiments of the compositions of the disclosure, the nucleic acid sequence further comprises: (e) a sequence encoding a 5′ untranslated region (UTR). In some embodiments, the sequence encoding the 5′ UTR comprises a sequence isolated or derived from a naturally occurring sequence. In some embodiments, the sequence encoding the 5′ UTR comprises a sequence isolated or derived from a non-naturally-occurring sequence. In some embodiments, the sequence encoding the 5′ UTR comprises a sequence isolated or derived from a mammalian sequence. In some embodiments, the sequence encoding the 5′ UTR comprises a sequence isolated or derived from a human sequence. In some embodiments, the sequence encoding the 5′ UTR comprises a sequence isolated or derived from a viral sequence.

In some embodiments of the compositions of the disclosure, the nucleic acid sequence further comprises: (f) a sequence encoding an intron, and (g) a sequence encoding an exon, wherein the sequence encoding the intron and the sequence encoding the exon are operably linked. In some embodiments, the intron is located between the sequence encoding the VMD2 promoter and the sequence encoding the exon, wherein the sequence encoding the exon is located between the sequence encoding the intron and the sequence encoding the 5′ UTR, and wherein the sequence encoding the intron is spliced by a mammalian cell. In some embodiments, the sequence encoding the exon comprises a sequence isolated or derived from a mammalian gene. In some embodiments, the sequence encoding the exon comprises a sequence isolated or derived from a rabbit (Oryctolagus cuniculus) beta globin gene. In some embodiments, the sequence encoding the intron comprises a non-naturally occurring sequence. In some embodiments, the sequence encoding the intron comprises a fusion sequence. In some embodiments, the sequence encoding the intron comprises a sequence encoding a splice donor site, and a sequence encoding a splice branch point and acceptor site. In some embodiments, the sequence encoding the splice donor site comprises a sequence isolated or derived from a vertebrate gene. In some embodiments, the sequence encoding the splice donor site comprises a sequence isolated or derived from a chicken (Gallus gallus) beta actin gene (CBA). In some embodiments, the sequence encoding the splice branch point and acceptor site comprises a sequence isolated or derived from a vertebrate gene. In some embodiments, the sequence encoding the splice branch point and acceptor site comprises a sequence isolated or derived from a rabbit (Oryctolagus cuniculus) beta globin gene.

In some embodiments of the compositions of the disclosure, the sequence encoding the 5′ UTR comprises a sequence encoding a Kozak sequence or a portion thereof. In some embodiments, the sequence encoding a Kozak sequence has at least 50% identity to the nucleic acid sequence of GCCRCCATGG. In some embodiments, the sequence encoding a Kozak sequence comprises or consists of the nucleic acid sequence of GGCACCATGA.

In some embodiments of the compositions of the disclosure, the sequence encoding the human VMD2 promoter comprises or consists of

| (SEQ ID NO: 1) | |
|---|---|
| 1 | AATTCTGTCA TTTTACTAGG GTGATGAAAT TCCCAAGCAA CACCATCCTT TTCAGATAAG |
| 61 | GGCACTGAGG CTGAGAGAGG AGCTGAAACC TACCCGGGGT CACCACACAC AGGTGGCAAG |
| 121 | GCTGGGACCA GAAACCAGGA CTGTTGACTG CAGCCCGGTA TTCATTCTTT CCATAGCCCA |
| 181 | CAGGGCTGTC AAAGACCCCA GGGCCTAGTC AGAGGCTCCT CCTTCCTGGA GAGTTCCTGG |
| 241 | CACAGAAGTT GAAGCTCAGC ACAGCCCCCT AACCCCCAAC TCTCTCTGCA AGGCCTCAGG |
| 301 | GGTCAGAACA CTGGTGGAGC AGATCCTTTA GCCTCTGGAT TTTAGGGCCA TGGTAGAGGG |
| 361 | GGTGTTGCCC TAAATTCCAG CCCTGGTCTC AGCCCAACAC CCTCCAAGAA GAAATTAGAG |
| 421 | GGGCCATGGC CAGGCTGTGC TAGCCGTTGC TTCTGAGCAG ATTACAAGAA GGGACTAAGA |
| 481 | CAAGGACTCC TTTGTGGAGG TCCTGGCTTA GGGAGTCAAG TGACGGCGGC TCAGCACTCA |
| 541 | CGTGGGCAGT GCCAGCCTCT AAGAGTGGGC AGGGGCACTG GCCACAGAGT CCCAGGGAGT |
| 601 | CCCACCAGCC TAGTCGCCAG ACC. |

In some embodiments of the compositions of the disclosure, the sequence encoding the CAG promoter comprises or consists of

| (SEQ ID NO: 2) | |
|---|---|
| 1 | CCATTGACGT CAATAATGAC GTATGTTCCC ATAGTAACGC CAATAGGGAC TTTCCATTGA |
| 61 | CGTCAATGGG TGGAGTATTT ACGGTAAACT GCCCACTTGG CAGTACATCA AGTGTATCAT |
| 121 | ATGCCAAGTA CGCCCCCTAT TGACGTCAAT GACGGTAAAT GGCCCGCCTG GCATTATGCC |
| 181 | CAGTACATGA CCTTATGGGA CTTTCCTACT TGGCAGTACA TCTACGTATT AGTCATCGCT |
| 241 | ATTACCATGG TCGAGGTGAG CCCCACGTTC TGCTTCACTC TCCCCATCTC CCCCCCCTCC |
| 301 | CCACCCCCAA TTTTGTATTT ATTTATTTTT TAATTATTTT GTGCAGCGAT GGGGGCGGGG |
| 361 | GGGGGGGGGG GGCGCGCGCC AGGCGGGGCG GGGCGGGGCG AGGGGCGGGG CGGGGCGAGG |
| 421 | CGGAGAGGTG CGGCGGCAGC CAATCAGAGC GGCGCGCTCC GAAAGTTTCC TTTTATGGCG |
| 481 | AGGCGGCGGC GGCGGCGGCC CTATAAAAAG CGAAGCGCGC GGCGGGCGGG AGTCGCTGCG |
| 541 | CGCTGCCTTC GCCCCGTGCC CCGCTCCGCC GCCGCCTCGC GCCGCCCGCC CCGGCTCTGA |
| 601 | CTGACCGCGT TACTCCCACA GGTGAGCGGG CGGGACGGCC CTTCTCCTCC GGGCTGTAAT |
| 661 | TAGCGCTTGG TTTAATGACG GCTTGTTTCT TTTCTGTGGC TGCGTGAAAG CCTTGAGGGG |
| 721 | CTCCGGGAGG GCCCTTTGTG CGGGGGGAGC GGCTCGGGGC TGTCCGCGGG GGGACGGCTG |
| 781 | CCTTCGGGGG GGACGGGGCA GGGCGGGGTT CGGCTTCTGG CGTGTGACCG GCGGCTCTAG |
| 841 | AGCCTCTGCT AACCATGTTC ATGCCTTCTT CTTTTTCCTA CAGCTCCTGG GCAACGTGCT |
| 901 | GGTTATTGTG CTGTCTCATC ATTTTGGCAA AGAATTGGAT C. |

In some embodiments of the compositions of the disclosure, the sequence encoding the human BEST1 protein comprises or consists of

| (SEQ ID NO: 3) | |
|---|---|
| 1 | ATGACCATCA CTTACACAAG CCAAGTGGCT AATGCCCGCT TAGGCTCCTT CTCCCGCCTG |
| 61 | CTGCTGTGCT GGCGGGGCAG CATCTACAAG CTGCTATATG GCGAGTTCTT AATCTTCCTG |
| 121 | CTCTGCTACT ACATCATCCG CTTTATTTAT AGGCTGGCCC TCACGGAAGA ACAACAGCTG |
| 181 | ATGTTTGAGA AACTGACTCT GTATTGCGAC AGCTACATCC AGCTCATCCC CATTTCCTTC |
| 241 | GTGCTGGGCT TCTACGTGAC GCTGGTCGTG ACCCGCTGGT GGAACCAGTA CGAGAACCTG |
| 301 | CCGTGGCCCG ACCGCCTCAT GAGCCTGGTG TCGGGCTTCG TCGAAGGCAA GGACGAGCAA |
| 361 | GGCCGGCTGC TGCGGCGCAC GCTCATCCGC TACGCCAACC TGGGCAACGT GCTCATCCTG |
| 421 | CGCAGCGTCA GCACCGCAGT CTACAAGCGC TTCCCCAGCG CCCAGCACCT GGTGCAAGCA |
| 481 | GGCTTTATGA CTCCGGCAGA ACACAAGCAG TTGGAGAAAC TGAGCCTACC ACACAACATG |
| 541 | TTCTGGGTGC CCTGGGTGTG GTTTGCCAAC CTGTCAATGA AGGCGTGGCT TGGAGGTCGA |
| 601 | ATCCGGGACC CTATCCTGCT CCAGAGCCTG CTGAACGAGA TGAACACCTT GCGTACTCAG |
| 661 | TGTGGACACC TGTATGCCTA CGACTGGATT AGTATCCCAC TGGTGTATAC ACAGGTGGTG |
| 721 | ACTGTGGCGG TGTACAGCTT CTTCCTGACT TGTCTAGTTG GGCGGCAGTT TCTGAACCCA |
| 781 | GCCAAGGCCT ACCCTGGCCA TGAGCTGGAC CTCGTTGTGC CCGTCTTCAC GTTCCTGCAG |
| 841 | TTCTTCTTCT ATGTTGGCTG GCTGAAGGTG GCAGAGCAGC TCATCAACCC CTTTGGAGAG |
| 901 | GATGATGATG ATTTTGAGAC CAACTGGATT GTCGACAGGA ATTTGCAGGT GTCCCTGTTG |
| 961 | GCTGTGGATG AGATGCACCA GGACCTGCCT CGGATGGAGC CGGACATGTA CTGGAATAAG |
| 1021 | CCCGAGCCAC AGCCCCCCTA CACAGCTGCT TCCGCCCAGT TCCGTCGAGC CTCCTTTATG |
| 1081 | GGCTCCACCT TCAACATCAG CCTGAACAAA GAGGAGATGG AGTTCCAGCC CAATCAGGAG |
| 1141 | GACGAGGAGG ATGCTCACGC TGGCATCATT GGCCGCTTCC TAGGCCTGCA GTCCCATGAT |
| 1201 | CACCATCCTC CCAGGGCAAA CTCAAGGACC AAACTACTGT GGCCCAAGAG GGAATCCCTT |
| 1261 | CTCCACGAGG GCCTGCCCAA AAACCACAAG GCAGCCAAAC AGAACGTTAG GGGCCAGGAA |
| 1321 | GACAACAAGG CCTGGAAGCT TAAGGCTGTG GACGCCTTCA AGTCTGCCCC ACTGTATCAG |
| 1381 | AGGCCAGGCT ACTACAGTGC CCCACAGACG CCCCTCAGCC CCACTCCCAT GTTCTTCCCC |
| 1441 | CTAGAACCAT CAGCGCCGTC AAAGCTTCAC AGTGTCACAG GCATAGACAC CAAAGACAAA |
| 1501 | AGCTTAAAGA CTGTGAGTTC TGGGGCCAAG AAAAGTTTTG AATTGCTCTC AGAGAGCGAT |
| 1561 | GGGGCCTTGA TGGAGCACCC AGAAGTATCT CAAGTGAGGA GGAAAACTGT GGAGTTTAAC |
| 1621 | CTGACGGATA TGCCAGAGAT CCCCGAAAAT CACCTCAAAG AACCTTTGGA ACAATCACCA |
| 1681 | ACCAACATAC ACACTACACT CAAAGATCAC ATGGATCCTT ATTGGGCCTT GGAAAACAGG |
| 1741 | GATGAAGCAC ATTCCTAA. |

The disclosure provides a vector comprising a composition of the disclosure. In some embodiments, the vector is a plasmid.

The disclosure provides a delivery vector comprising the vector of the disclosure. In some embodiments, the delivery vector is a viral delivery vector. In some embodiments, the delivery vector comprises a single stranded viral genome. In some embodiments, the delivery vector comprises a double stranded viral genome. In some embodiments, the delivery vector comprises an RNA molecule.

The disclosure provides a delivery vector comprising the vector of the disclosure. In some embodiments, the delivery vector comprises a sequence isolated or derived from an adeno-associated virus (AAV) vector. In some embodiments, the delivery vector comprises a sequence isolated or derived from an AAV vector of serotype AAV1, AAV2, AAV3, AAV4, AAV5, AAV6, AAV7, AAV8, AAV9, AAV10, AAV11 or any combination thereof. In some embodiments, the delivery vector comprises a sequence isolated or derived from an AAV vector of serotype AAV2. In some embodiments, the delivery vector comprises a sequence isolated or derived from an AAV vector of serotype AAV8. In some embodiments, the delivery vector comprises a sequence encoding a first inverted terminal repeat (ITR) and a second ITR isolated or derived from an AAV vector of serotype AAV2 and a sequence encoding a viral gene isolated or derived from an AAV vector of serotype AAV2. In some embodiments, the delivery vector comprises a sequence encoding a first inverted terminal repeat (ITR) and a second ITR isolated or derived from an AAV vector of serotype AAV8 and a sequence encoding a viral gene isolated or derived from an AAV vector of serotype AAV8. In some embodiments, the delivery vector comprises a sequence encoding a first inverted terminal repeat (ITR) and a second ITR isolated or derived from an AAV vector of serotype AAV2 and a sequence encoding a viral gene isolated or derived from an AAV vector of serotype AAV8.

The disclosure provides a pharmaceutical composition comprising a composition of the disclosure and a pharmaceutically-acceptable carrier. In some embodiments, the pharmaceutically-acceptable carrier comprises TMN200.

The disclosure provides a pharmaceutical composition comprising a vector of the disclosure a pharmaceutically acceptable carrier. In some embodiments, the pharmaceutically acceptable carrier comprises TMN200.

The disclosure provides a pharmaceutical composition comprising a delivery vector of the disclosure and a pharmaceutically acceptable carrier. In some embodiments, the pharmaceutically acceptable carrier comprises TMN200.

The disclosure provides a cell comprising a composition of the disclosure. The disclosure provides a cell comprising a vector of the disclosure. The disclosure provides a cell comprising a delivery vector of the disclosure. The disclosure provides a cell comprising a pharmaceutical composition of the disclosure. In some embodiments, the cell is a mammalian cell. In some embodiments, the mammalian cell is a non-human primate cell, a rodent cell, a mouse cell, a rat cell or a rabbit cell. In some embodiments, the cell is a human cell. In some embodiments, the human cell is a neuronal cell, a glial cell, a retinal cell, a photoreceptor cell, a rod cell, a cone cell or a cuboidal cell of the retinal pigment epithelium (RPE). In some embodiments, the human cell is a photoreceptor cell. In some embodiments, the human cell is an HEK293 cell or an ARPE19 cell. In some embodiments, the human cell is isolated or derived from an RPE of a human retina. In some embodiments, the cell is in vivo, in vitro, ex vivo or in situ.

The disclosure provides a method of treating macular dystrophy in a subject in need thereof, comprising administering to the subject a therapeutically effective amount of a composition of the disclosure.

The disclosure provides a method of treating macular dystrophy in a subject in need thereof, comprising administering to the subject a therapeutically effective amount of a composition comprising the vector of the disclosure.

The disclosure provides a method of treating macular dystrophy in a subject in need thereof, comprising administering to the subject a therapeutically effective amount of a composition comprising the delivery vector of the disclosure.

In some embodiments of the methods of the disclosure, the subject is a human. In some embodiments, the subject is a non-human primate, a dog, a cat, a rodent, a mouse, a rat, or a rabbit. In some embodiments, the subject has macular dystrophy.

In some embodiments of the methods of the disclosure, the subject has a mutation in one or both copies of a BEST1 gene. In some embodiments, the mutation is heritable as a dominant mutation. In some embodiments, the dominant mutation causes Best Vitelliform Macular Dystrophy (BVMD) in the subject. In some embodiments, the mutation is heritable as a recessive mutation. In some embodiments, the recessive mutation causes Autosomal Recessive Bestrophinopathy (ARB) in the subject. In some embodiments, the mutation occurs in a coding sequence of one or both copies of a BEST1 gene. In some embodiments, the mutation occurs in a non-coding sequence of one or both copies of a BEST1 gene. In some embodiments, the mutation comprises a substitution, an insertion, a deletion, an inversion, a translocation, a frameshift, or a combination thereof in one both copies of a BEST1 gene.

In some embodiments of the methods of the disclosure, administering comprises an injection or an infusion via a subretinal, a suprachoroidal or an intravitreal route. In some embodiments, administering comprises an injection or an infusion via a subretinal route. In some embodiments, administering comprises a two-step injection or a two-step infusion via a subretinal route.

In some embodiments of the methods of the disclosure, the therapeutically effective amount is formulated in a volume of between 10 and 200 μL, inclusive of the endpoints. In some embodiments, the therapeutically effective amount is formulated in a volume of between 10 and 50 between 50 and 100 μL, between 100 and 150 μL or between 150 and 200 μL, inclusive of the endpoints, for each range. In some embodiments, the therapeutically effective amount is formulated in a volume of between 70 and 120 μL, inclusive of the endpoints, and wherein the administering comprises an injection or an infusion via a subretinal route. In some embodiments, the therapeutically effective amount is formulated in a volume of 100 μL and wherein the administering comprises an injection or an infusion via a subretinal route.

In some embodiments of the methods of the disclosure, the therapeutically effective amount comprises a concentration of an AAV delivery vector of at least 1×10¹⁰ DRP/mL, at least 1×10¹¹ DRP/mL, at least 1×10¹² DRP/mL, at least 2×10¹¹ DRP/mL, at least 5×10¹² DRP/mL or at least 1.5×10¹³ DRP/mL. In some embodiments, the therapeutically effective amount comprises a concentration of an AAV delivery vector of at least 2×10¹¹ DRP/mL, at least 5×10¹² DRP/mL or at least 1.5×10¹³ DRP/mL. In some embodiments, the therapeutically effective amount comprises a concentration of an AAV delivery vector of at least 5×10¹¹ DRP/mL. In some embodiments, the therapeutically effective amount comprises a concentration of an AAV delivery vector of at least 1.5×10¹¹ DRP/mL.

In some embodiments of the methods of the disclosure, the therapeutically effective amount comprises a dose of 2×10⁸ genome particles (gp), 5×10⁸ gp, 1.5×10⁹ gp, 2×10⁹ gp, 5×10⁹ gp, 2×10¹⁰ gp, 5×10¹⁰ gp, 6×10¹⁰ gp, 1.2×10¹¹ gp, 1.5×10¹¹ gp, 2×10¹¹ gp, 4.5×10¹¹ gp, 5×10¹¹ gp, 1.2×10¹² gp, 1.5×10¹² gp, 2×10¹² gp or 5×10¹² gp. In some embodiments, the subject is a mouse and wherein the therapeutically effective amount comprises a dose of 5×10⁸ gp, 1.5×10⁹ gp or 5×10⁹ gp. In some embodiments, the subject is a non-human primate and wherein the therapeutically effective amount comprises a dose of 1.2×10¹¹ gp, 4.5×10¹¹ gp or 1.2×10¹² gp of AAV viral particles. In some embodiments, the subject is human and wherein the therapeutically effective amount comprises a dose of 5×10¹⁰ gp, 1.5×10¹¹ gp, 5×10¹¹ gp or 1.5×10¹² gp of AAV viral particles.

In some embodiments of the methods of the disclosure, the composition further comprises a TMN200 buffer.

The disclosure provides a composition of the disclosure for use in treating macular dystrophy in a subject in need thereof.

The disclosure provides a vector of the disclosure for use in treating macular dystrophy in a subject in need thereof.

The disclosure provides a delivery vector of the disclosure for use in treating macular dystrophy in a subject in need thereof.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1A is a map of a plasmid encoding VMD2.IntEx.BEST1.WPRE.pA construct with AAV2 ITRs. FIG. 1B is a map of a plasmid encoding VMD2.IntEx.BEST1.WPRE.pA construct with AAV2 ITRs.

FIG. 2A is a map of a plasmid encoding VMD2.BEST1.WPRE.pA construct with AAV2 ITRs. FIG. 2B is a map of a plasmid encoding VMD2.BEST1.WPRE.pA construct with AAV2 ITRs.

FIG. 3A-3C are a series of three maps of two plasmids encoding CAG.BEST1.WPRE.pA with AAV2 ITRs. FIG. 3A is a map of a CAG.BEST.WPRE.pA plasmid with an AmpR selectable marker. FIG. 3B is a map of a CAG.BEST.WPRE.pA plasmid with an AmpR selectable marker. FIG. 3C is a map of a CAG.BEST.WPRE.pA plasmid with a KanR selectable marker and a stuffer sequence.

FIG. 4 is a map of a plasmid encoding VMD2.GFP.WPRE.pA with AAV2 ITRs.

FIG. 5 is a map of a plasmid encoding VMD2.Int.Ex.GFP.WPRE.pA with AAV2 ITRs.

FIG. 6A-6B are each a series of images, 6 images (FIG. 6A) and 3 images (FIG. 6B), showing BEST1 expression in HEK293 cells transduced with AAV.CAG.BEST1.pA, AAV.CAG.BEST1.WPRE.pA and an untransduced control. Cells are stained with Hoechst blue dye and anti-hBestrophin-1. Bestrophin-1 protein is localized throughout the cytosol

FIG. 7A-7B is a picture of a Western Blot (FIG. 7A) and a bar graph (FIG. 7B), respectively, showing the expression of Bestrophin-1 protein and a beta-actin control in HEK293 cells transduced with AAV.CAG.BEST1.pA (sample 1) or AAV.CAG.BEST1.WPRE.pA (sample 2) or a negative control (sample 3). Plasmid-transfected HEK293 cells were used as a positive control. FIG. 7B shows the quantification of Bestrophin-1 protein expression in HEK293 cells transduced with AAV.CAG.BEST1.pA (n=9) or AAV.CAG.BEST1.WPRE.pA (n=9) or an untransduced negative control (n=8). The Y-axis shows the normalized LiCor Value. Error bars are ±SEM. *** indicates p<0.001 when compared to the un-transduced control.

FIG. 8A-8B is a single plot (FIG. 8A) and a series of four plots (FIG. 8B), respectively, showing whole-cell patch clamp recording data from HEK293 cells transduced with AAV2/2 CAG.BEST1.pA, AAV2/2 CAG.BEST1.WPRE.pA or AAV2/2 CAG.GFP.WPRE.pA vectors, as well as an untransduced control. In FIG. 8A, Current (pA) is plotted on the X-axis from −140 to 500 in increments of 20, while Voltage (in mV) is plotted on the Y-axis from −200 to 500 in units of 100. In FIG. 8B, the current waveforms are shown. Current (I)/Voltage (V) plots of HEK293 transduced with the different vectors and an untransduced control are shown, clockwise from the top left: AAV2/2 CAG.BEST1.pA, AAV2/2 CAG.BEST1.WPRE.pA, untransduced control and AAV2/2 CAG.GFP.WPRE.pA. The inset scale bar (center) shows 250 pA on the Y-axis and 100 milliseconds on the X-axis.

FIG. 9A-9B are a bar graph (FIG. 9A) and a dot plot (FIG. 9B) showing the chord conductance of HEK293 cells transduced with AAV2/2 CAG.BEST1.pA (n=10), AAV2/2 CAG.BEST1.WPRE.pA (n=10), AAV2/2 CAG.GFP.WPRE.pA (n=11) and an untransduced control (n=10). Chord conductance is plotted on the Y-axis from 0 to 10 in units of 2. **** indicates p<0.0001, * indicates p<0.05, ns stands for not significant.

FIG. 10A-10B are a pair of flow charts showing two embodiments of an experimental procedure for assaying BEST1 expression in differentiated ARPE19 cells. FIG. 10A shows an experimental procedure for assaying BEST1 expression in transfected differentiated ARPE19 cells. FIG. 10B shows an experimental procedure for assaying BEST1 expression in transfected and/or transduced differentiated ARPE19 cells.

FIG. 11A-11B is a series of 16 images (FIG. 11A) and 6 images (FIG. 11B) showing BEST1 and ZO-1 immunostaining of transfected ARPE19 cells that were differentiated for 1 month. (FIG. 11A) The rows, top to bottom show ARPE19 cells with the following constructs: untransfected control, CAG.BEST1.WPRE, VMD2.BEST1.WPRE and VMD2.IntEx.BEST1.WPRE. The columns from left to right show: nuclei stained with Hoechst in blue, ZO-1 staining in green (ZO-1 is a marker of the cytoplasmic membrane surface of intercellular tight junctions), BEST1 in red, and a merged image (Hoechst, ZO-1, BEST1). Scale bars show 100 microns (μm). (FIG. 11B) Shown in the top row are ARPE19 cells transfected with VMD2.BEST1.WPRE.pA. Shown in the bottom row are ARPE19 cells transfected with VMD2.IntEx.BEST1.WPRE.pA. The images from left to right show ZO-1 (green) and BEST1 (red), and a merged image (Hoechst, ZO-1 and BEST1). The scale bar in the merged images indicates 25 μm.

FIG. 12A-12B is a series of 16 images (FIG. 12A) and 9 images (FIG. 12B) showing BEST1 and ZO-immunostaining of transfected ARPE19 cells that were differentiated for 3 months. (FIG. 12A) The rows, top to bottom show ARPE19 cells with the following constructs: untransfected control, CAG.BEST1.WPRE, VMD2.BEST1.WPRE and VMD2.IntEx.BEST1.WPRE. The columns from left to right show: nuclei stained with Hoechst in blue, ZO-1 staining in green, BEST1 in red, and a merged image (Hoechst, ZO-1, BEST1). Scale bars show 100 microns (μm). (FIG. 12B) Representative images of FIG. 12A at higher magnification. The rows from top to bottom show CAG.BEST1.WPRE, VMD2.BEST1.WPRE and VMD2.IntEx.BEST1.WPRE. The columns from left to right show staining for ZO-1 in green, BEST1 in red and a merged image including Hoechst in blue. The scale bar in the merged images indicates 25 μm.

FIG. 13A-13B shows two series of 8 images each showing GFP fluorescence in ARPE19 cells differentiated for 4 months, pre-treated with 400 nM doxorubicin and transduced with (FIG. 13A) AAV2/2.CAG.GFP.WPRE or (FIG. 13B) AAV2/2.VMD2.InEx.GFP.WPRE at 3 different multiplicities of infection (MOI). The MOIs used were 2, 4 and 8×10⁴ genome particles (gp)/cell. The scale bars in the negative control (untransduced and untreated cells) indicates 50 am. The top row in each panel indicates untreated control cells, the bottom row are cells pre-treated with 400 nM doxorubicin.

FIG. 14 is a series of 20 images showing BEST1 and ZO-1 immunostaining of ARPE19 cells differentiated for 4 months, pre-treated with 400 nM doxorubicin and transduced with AAV2/2.CAG.BEST1.WPRE and AAV2/2.VMD2.InEx.BEST1.WPRE at two different MOIs: 1 and 4×10⁴ gp/cell. The rows, top to bottom show ARPE19 cells with the following viral vectors: untransduced control, AAV2/2.CAG.BEST1.WPRE at a MOI 10,000 gp/cell, AAV2/2.CAG.BEST1.WPRE at a MOI 40,000 gp/cell, AAV2/2.VMD2.InEx.BEST1.WPRE at a MOI 10,000 gp/cell and AAV2/2.VMD2.InEx.BEST1.WPRE at a MOI 40,000 gp/cell. The columns from left to right show: nuclei stained with Hoechst in blue, ZO-1 staining in green, BEST1 in red, and a merged image (Hoechst, ZO-1, BEST1). Scale bars show 50 m.

FIG. 15 is a table outlining a 4/8 week in vivo pilot study protocol in mice.

FIG. 16 is a series of 6 optical coherence tomography (OCT) images of mouse eyes four weeks after being injected with sham, VMD2.BEST1.WPRE or VMD2.IntEx.BEST1.WPRE AAV constructs. The columns, from left to right, show mice injected with a sham, with VMD2.BEST1.WPRE and with VMD2.IntEx.BEST1.WPRE AAV constructs.

FIG. 17 is a series of 3 OCT images of mouse eyes four weeks after being injected with, from left to right: sham, VMD2.BEST1.WPRE or VMD2.IntEx.BEST1.WPRE AAV constructs. Indicated morphological structures are the retinal ganglion cell (RGC), inner plexiform layer (IPL), the inner nuclear layer (INL), the outer plexiform layer (OPL), the outer nuclear layer (ONL), the retinal pigmented epithelium (RPE). Blue and red arrows indicate retinal thicknesses.

FIG. 18 is a series of 12 OCT images of mouse eyes four and eight weeks after being injected with sham, VMD2.BEST1.WPRE or VMD2.IntEx.BEST1.WPRE AAV constructs (columns, from left to right). Mid-sagittal and off-center views are shown in alternating rows. The top two rows are animals imaged at 4 weeks post injection, and the bottom two rows are animals imaged at 8 weeks post injection.

FIG. 19 is a series of 12 fluorescent microscopy images of mouse eyes four weeks after being injected with sham, VMD2.BEST1.WPRE or VMD2.IntEx.BEST1.WPRE AAV constructs and stained with anti BEST1 (green), anti Rhodopsin (red) and DAPI (blue). The rows show, from top to bottom, sham injected eyes, eyes injected VMD2.BEST1.WPRE or VMD2.IntEx.BEST1.WPRE AAV particles. The columns, from left to right, show anti BEST1 (green), anti Rhodopsin (red), DAPI (blue) and a merged image. The retinal pigment epithelium (RPE), photoreceptors (PR) and retinal ganglion cells (RGC) are indicated at bottom.

FIG. 20 is a series of 12 images of mouse eyes eight weeks after being injected with, in columns from left to right: sham, VMD2.BEST1.WPRE or VMD2.IntEx.BEST1.WPRE AAV particles and stained for BEST1 (green), Rhodopsin (red) and DAPI (blue). The rows, from top to bottom, are a merged image, anti-BEST1 (also called huBEST1), anti-Rhodopsin and a bright field image.

FIG. 21 is an image of a western blot showing BEST1 protein expression in dissected mouse RPE and choroid complex four weeks after injection with sham, CAG.BEST1.WPRE, VMD2.BEST1.WPRE or VMD2.IntEx.BEST1.WPRE AAV constructs. The blue arrow indicates a recombinant human Bestrophin-1 protein, while the red arrow indicates the suggested size of the BEST1 protein. CAG.BEST1.WPRE was used as a control. CAG is a strong promoter with a constitutive expression in mammalian cells. It is an hybrid between the cytomegalovirus (CMV) enhancer element, the chicken beta-actin promoter (CBA) and the splice acceptor of the rabbit beta-globin gene.

FIG. 22 is a table outlining a 4/13 week in vivo proof of concept (PoC) study protocol in mice.

FIG. 23 is a series of 20 OCT images of mouse eyes four weeks and 13 weeks after being injected with sham, VMD2.IntEx.BEST1.WPRE or VMD2.BEST1.WPRE AAV constructs at two different dosages (1×10⁸ GC/μL/eye and 1×10⁹ GC/μL/eye). Mid-sagittal (top row) and off-center (bottom row) views are shown in alternating rows.

FIG. 24 is a series of 20 microscopy images of mouse eyes four weeks after being injected with sham, VMD2.IntEx.BEST1.WPRE or VMD2.BEST1.WPRE AAV constructs at two different dosages (1×10⁸ GC/μL/eye and 1×10⁹ GC/μL/eye), and stained with anti-BEST1 (huBEST1, green), anti-Rhodopsin (red) and DAPI (blue). Also shown are bright field images (bottom row). The columns, from left to right, show VMD2.IntEx.BEST1.WPRE at 1×10⁸ GC/μL/eye, VMD2.IntEx.BEST1.WPRE at 1×10⁹ GC/μL/eye, VMD2.BEST1.WPRE at 1×10⁸ GC/μL/eye and VMD2.BEST1.WPRE at 1×10⁹ GC/μL/eye. Rows, from top to bottom show: a merged image, anti-BEST1, anti-Rhodopsin and bright field. Anatomical structures indicated in the upper left image are the inner nuclear layer (INL), the outer nuclear layer (ONL), the outer segment (OS), the retinal pigment epithelium (RPE) and the choroid.

FIG. 25A-25B are a pair of images of western blots looking at BEST1 protein expression in cells or injected mouse RPE and choroid complex. FIG. 25A is a western blot showing the expression of Bestrophin-1 protein and a beta-actin control in HEK293 and ARPE-19 cells transfected with pCAG.BEST1.WPRE, pVMD2.BEST1.WPRE and pVMD2.InEx.BEST1.WPRE or an untransfected sample as negative control. FIG. 25B is a western blot showing BEST1 protein in isolated RPE and choroid samples from mice injected with either a high does (1×10⁹ GC/μL/eye) or low dose (1×10⁸ GC/μL/eye) of either VMD2.IntEx.BEST1.WPRE or VMD2.BEST1.WPRE AAV particles.

FIG. 26 is a table showing a study design for assaying human BEST1 expression by immunohistochemistry and western blot in mice injected with AAV2/2. VMD2.InEx.BEST1.WPRE.

FIG. 27 is a table showing a protocol for a proposed good laboratory practice (GLP) study to assess potential toxicity in mice.

FIG. 28 is a table showing a protocol for the evaluation of toxicity assessment study materials at 4 weeks.

FIG. 29 is a table showing a protocol for a proposed good laboratory practice (GLP) study to assess potential toxicity in non-human primates.

FIG. 30 is a table showing a dosing regimen in mouse, non-human primate and human equivalent doses in genome particles (gp) using a BEST1 AAV viral vector of the disclosure. The BEST1 AAV viral vector for the proposed doses is at a concentration of 2×10¹² DRP/mL and made according to current good manufacturing practice (GMP) standards.

FIG. 31 is a table showing a dosing regimen and the required concentrations of DNAse resistant particles (DRP) and number of genome particles (gp) per dose in mouse, non-human primate and human of a BEST1 AAV viral vector of the disclosure.

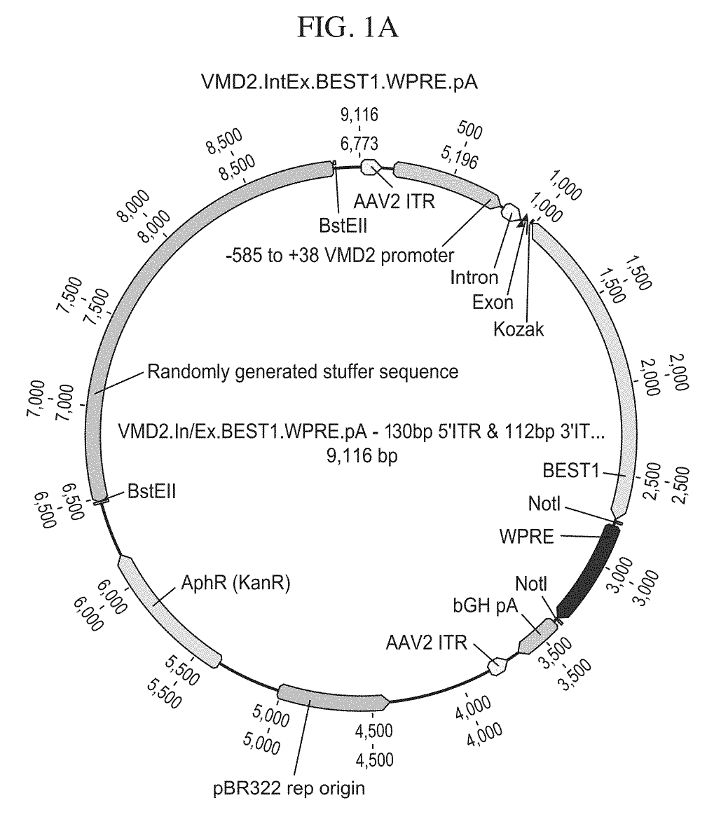

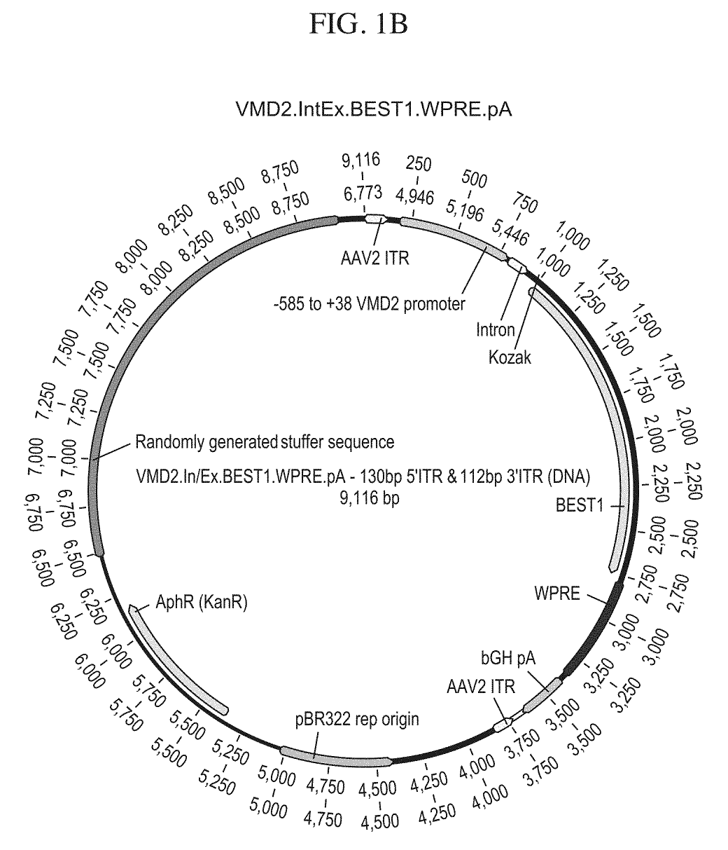

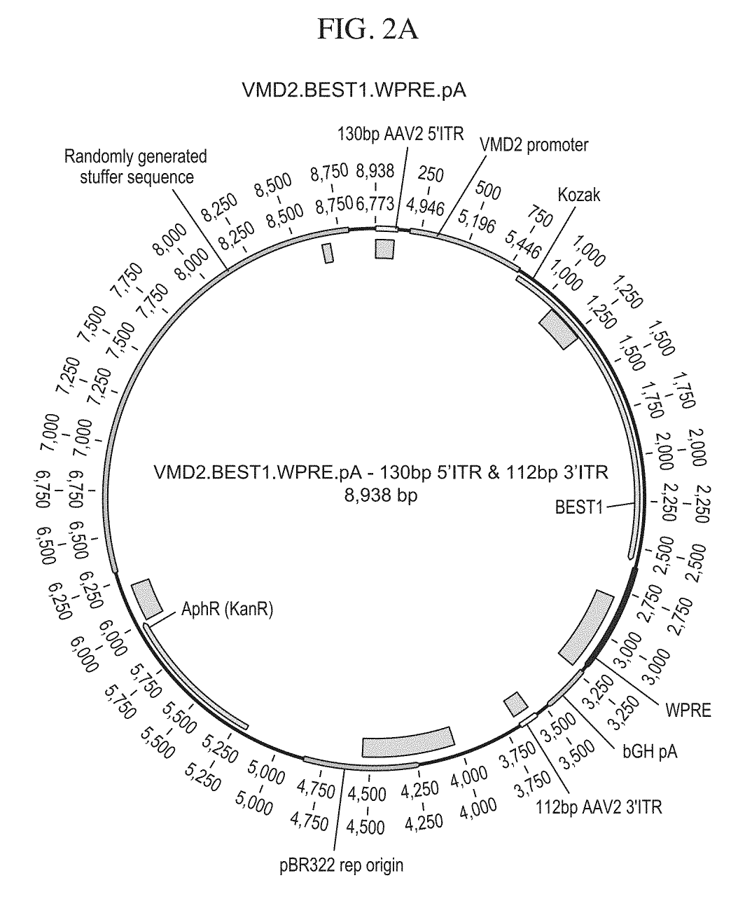

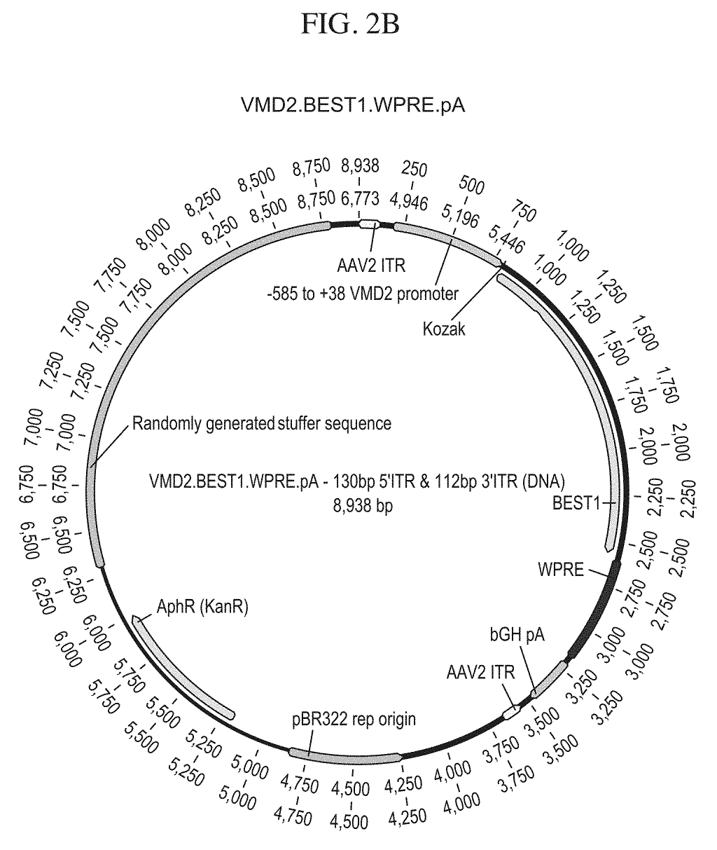

### DETAILED DESCRIPTION

The disclosure relates to the finding that in many cases macular degeneration may be caused by mutations in or the abnormal function of the protein Bestrophin-1 (BEST1, also known as VMD2). The macula is a region near the center of the retina, and is responsible for central, high-resolution color vision. The fovea, located near the center of the macula, contains the largest concentration of cone cell photoreceptors in the eye. Mutations in a gene called Bestrophin-1 (BEST1, or human BEST1 (hBEST1), also known as VMD2) are associated with at least five distinct retinal degeneration diseases, called bestrinopathies. Bestrinopathies comprise best vitelliform macular dystrophy (BVMD), autosomal recessive bestrophinopathy, adult-onset vitelliform macular dystrophy, autosomal dominant vitreoretinochoroidopathy and retinitis pigmentosa. These mutations can be either dominant (for example, BVMD) or recessive. Best Vitelliform Macular Dystrophy (BVMD) and Autosomal Recessive Bestrophinopathy may cause macular degeneration with an onset in late childhood or adolescence. However, in some cases, macular degeneration begins in adulthood. However, regardless of age of onset, bestrinopathies can have a devastating effect on vision, and there is currently no known effective treatment. Given the key role that BEST1 function plays in bestrophinopathies, one approach to the treatment of bestophinopathy is to deliver a functional BEST1 protein to the affected cells of the patient.

### Bestrophin-1 (BEST1)

Bestrophin-1 (BEST1) is an integral membrane protein found primarily in the retinal pigment epithelium of the eye (RPE) and predominantly localizes to the basolateral plasma membrane. BEST1 protein is thought to function as an ion channel and a regulator of intracellular calcium signaling. Human BEST1 can be found in the NCBI database with accession numbers NP_004174.1 and NM_004183.3, the contents of which are incorporated by reference in their entirety herein.

In some embodiments of the compositions of the disclosure, a sequence encoding a BEST1 protein of the disclosure comprises or consists of an amino acid sequence having at least 90%, at least 95%, at least 96%, at least 97%, at least 98% or at least 99% identity to the sequence of:

| (SEQ ID NO: 4) | |
|---|---|
| 1 | MTITYTSQVA NARLGSFSRL LLCWRGSIYK LLYGEFLIFL LCYYIIRFIY RLALTEEQQL |
| 61 | MFEKLTLYCD SYIQLIPISF VLGFYVTLVV TRWWNQYENL PWPDRLMSLV SGFVEGKDEQ |
| 121 | GRLLRRTLIR YANLGNVLIL RSVSTAVYKR FPSAQHLVQA GFMTPAEHKQ LEKLSLPHNM |
| 181 | FWVPWVWFAN LSMKAWLGGR IRDPILLQSL LNEMNTLRTQ CGHLYAYDWI SIPLVYTQVV |
| 241 | TVAVYSFFLT CLVGRQFLNP AKAYPGHELD LVVPVFTFLQ FFFYVGWLKV AEQLINPFGE |
| 301 | DDDDFETNWI VDRNLQVSLL AVDEMHQDLP RMEPDMYWNK PEPQPPYTAA SAQFRRASFM |
| 361 | GSTFNISLNK EEMEFQPNQE DEEDAHAGII GRFLGLQSHD HHPPRANSRT KLLWPKRESL |
| 421 | LHEGLPKNHK AAKQNVRGQE DNKAWKLKAV DAFKSAPLYQ RPGYYSAPQT PLSPTPMFFP |
| 481 | LEPSAPSKLH SVTGIDTKDK SLKTVSSGAK KSFELLSESD GALMEHPEVS QVRRKTVEFN |
| 541 | LTDMPEIPEN HLKEPLEQSP TNIHTTLKDH MDPYWALENR DEAHS. |

In some embodiments of the compositions of the disclosure, a sequence encoding a BEST1 protein of the disclosure comprises or consists of the amino acid sequence:

| (SEQ ID NO: 4) | |
|---|---|
| 1 | MTITYTSQVA NARLGSFSRL LLCWRGSIYK LLYGEFLIFL LCYYIIRFIY RLALTEEQQL |
| 61 | MFEKLTLYCD SYIQLIPISF VLGFYVTLVV TRWWNQYENL PWPDRLMSLV SGFVEGKDEQ |
| 121 | GRLLRRTLIR YANLGNVLIL RSVSTAVYKR FPSAQHLVQA GFMTPAEHKQ LEKLSLPHNM |
| 181 | FWVPWVWFAN LSMKAWLGGR IRDPILLQSL LNEMNTLRTQ CGHLYAYDWI SIPLVYTQVV |
| 241 | TVAVYSFFLT CLVGRQFLNP AKAYPGHELD LVVPVFTFLQ FFFYVGWLKV AEQLINPFGE |
| 301 | DDDDFETNWI VDRNLQVSLL AVDEMHQDLP RMEPDMYWNK PEPQPPYTAA SAQFRRASFM |
| 361 | GSTFNISLNK EEMEFQPNQE DEEDAHAGII GRFLGLQSHD HHPPRANSRT KLLWPKRESL |
| 421 | LHEGLPKNHK AAKQNVRGQE DNKAWKLKAV DAFKSAPLYQ RPGYYSAPQT PLSPTPMFFP |
| 481 | LEPSAPSKLH SVTGIDTKDK SLKTVSSGAK KSFELLSESD GALMEHPEVS QVRRKTVEFN |
| 541 | LTDMPEIPEN HLKEPLEQSP TNIHTTLKDH MDPYWALENR DEAHS. |

In some embodiments of the compositions of the disclosure, a nucleic acid sequence encoding a BEST1 protein of the disclosure comprises or consists of a nucleic acid having at least 80%, at least 90%, at least 95%, at least 96%, at least 97%, at least 98% or at least 99% identity to the nucleic acid sequence of:

| (SEQ ID NO: 3) | |
|---|---|
| 1 | ATGACCATCA CTTACACAAG CCAAGTGGCT AATGCCCGCT TAGGCTCCTT CTCCCGCCTG |
| 61 | CTGCTGTGCT GGCGGGGCAG CATCTACAAG CTGCTATATG GCGAGTTCTT AATCTTCCTG |
| 121 | CTCTGCTACT ACATCATCCG CTTTATTTAT AGGCTGGCCC TCACGGAAGA ACAACAGCTG |
| 181 | ATGTTTGAGA AACTGACTCT GTATTGCGAC AGCTACATCC AGCTCATCCC CATTTCCTTC |
| 241 | GTGCTGGGCT TCTACGTGAC GCTGGTCGTG ACCCGCTGGT GGAACCAGTA CGAGAACCTG |
| 301 | CCGTGGCCCG ACCGCCTCAT GAGCCTGGTG TCGGGCTTCG TCGAAGGCAA GGACGAGCAA |
| 361 | GGCCGGCTGC TGCGGCGCAC GCTCATCCGC TACGCCAACC TGGGCAACGT GCTCATCCTG |
| 421 | CGCAGCGTCA GCACCGCAGT CTACAAGCGC TTCCCCAGCG CCCAGCACCT GGTGCAAGCA |
| 481 | GGCTTTATGA CTCCGGCAGA ACACAAGCAG TTGGAGAAAC TGAGCCTACC ACACAACATG |
| 541 | TTCTGGGTGC CCTGGGTGTG GTTTGCCAAC CTGTCAATGA AGGCGTGGCT TGGAGGTCGA |
| 601 | ATCCGGGACC CTATCCTGCT CCAGAGCCTG CTGAACGAGA TGAACACCTT GCGTACTCAG |
| 661 | TGTGGACACC TGTATGCCTA CGACTGGATT AGTATCCCAC TGGTGTATAC ACAGGTGGTG |
| 721 | ACTGTGGCGG TGTACAGCTT CTTCCTGACT TGTCTAGTTG GGCGGCAGTT TCTGAACCCA |
| 781 | GCCAAGGCCT ACCCTGGCCA TGAGCTGGAC CTCGTTGTGC CCGTCTTCAC GTTCCTGCAG |
| 841 | TTCTTCTTCT ATGTTGGCTG GCTGAAGGTG GCAGAGCAGC TCATCAACCC CTTTGGAGAG |
| 901 | GATGATGATG ATTTTGAGAC CAACTGGATT GTCGACAGGA ATTTGCAGGT GTCCCTGTTG |
| 961 | GCTGTGGATG AGATGCACCA GGACCTGCCT CGGATGGAGC CGGACATGTA CTGGAATAAG |
| 1021 | CCCGAGCCAC AGCCCCCCTA CACAGCTGCT TCCGCCCAGT TCCGTCGAGC CTCCTTTATG |
| 1081 | GGCTCCACCT TCAACATCAG CCTGAACAAA GAGGAGATGG AGTTCCAGCC CAATCAGGAG |
| 1141 | GACGAGGAGG ATGCTCACGC TGGCATCATT GGCCGCTTCC TAGGCCTGCA GTCCCATGAT |
| 1201 | CACCATCCTC CCAGGGCAAA CTCAAGGACC AAACTACTGT GGCCCAAGAG GGAATCCCTT |
| 1261 | CTCCACGAGG GCCTGCCCAA AAACCACAAG GCAGCCAAAC AGAACGTTAG GGGCCAGGAA |
| 1321 | GACAACAAGG CCTGGAAGCT TAAGGCTGTG GACGCCTTCA AGTCTGCCCC ACTGTATCAG |
| 1381 | AGGCCAGGCT ACTACAGTGC CCCACAGACG CCCCTCAGCC CCACTCCCAT GTTCTTCCCC |
| 1441 | CTAGAACCAT CAGCGCCGTC AAAGCTTCAC AGTGTCACAG GCATAGACAC CAAAGACAAA |
| 1501 | AGCTTAAAGA CTGTGAGTTC TGGGGCCAAG AAAAGTTTTG AATTGCTCTC AGAGAGCGAT |
| 1561 | GGGGCCTTGA TGGAGCACCC AGAAGTATCT CAAGTGAGGA GGAAAACTGT GGAGTTTAAC |
| 1621 | CTGACGGATA TGCCAGAGAT CCCCGAAAAT CACCTCAAAG AACCTTTGGA ACAATCACCA |
| 1681 | ACCAACATAC ACACTACACT CAAAGATCAC ATGGATCCTT ATTGGGCCTT GGAAAACAGG |
| 1741 | GATGAAGCAC ATTCCTAA. |

In some embodiments of the compositions of the disclosure, a nucleic acid sequence encoding a BEST1 protein of the disclosure comprises or consists of the nucleic acid sequence:

| (SEQ ID NO: 3) | |
|---|---|
| 1 | ATGACCATCA CTTACACAAG CCAAGTGGCT AATGCCCGCT TAGGCTCCTT CTCCCGCCTG |
| 61 | CTGCTGTGCT GGCGGGGCAG CATCTACAAG CTGCTATATG GCGAGTTCTT AATCTTCCTG |
| 121 | CTCTGCTACT ACATCATCCG CTTTATTTAT AGGCTGGCCC TCACGGAAGA ACAACAGCTG |
| 181 | ATGTTTGAGA AACTGACTCT GTATTGCGAC AGCTACATCC AGCTCATCCC CATTTCCTTC |
| 241 | GTGCTGGGCT TCTACGTGAC GCTGGTCGTG ACCCGCTGGT GGAACCAGTA CGAGAACCTG |
| 301 | CCGTGGCCCG ACCGCCTCAT GAGCCTGGTG TCGGGCTTCG TCGAAGGCAA GGACGAGCAA |
| 361 | GGCCGGCTGC TGCGGCGCAC GCTCATCCGC TACGCCAACC TGGGCAACGT GCTCATCCTG |
| 421 | CGCAGCGTCA GCACCGCAGT CTACAAGCGC TTCCCCAGCG CCCAGCACCT GGTGCAAGCA |
| 481 | GGCTTTATGA CTCCGGCAGA ACACAAGCAG TTGGAGAAAC TGAGCCTACC ACACAACATG |
| 541 | TTCTGGGTGC CCTGGGTGTG GTTTGCCAAC CTGTCAATGA AGGCGTGGCT TGGAGGTCGA |
| 601 | ATCCGGGACC CTATCCTGCT CCAGAGCCTG CTGAACGAGA TGAACACCTT GCGTACTCAG |
| 661 | TGTGGACACC TGTATGCCTA CGACTGGATT AGTATCCCAC TGGTGTATAC ACAGGTGGTG |
| 721 | ACTGTGGCGG TGTACAGCTT CTTCCTGACT TGTCTAGTTG GGCGGCAGTT TCTGAACCCA |
| 781 | GCCAAGGCCT ACCCTGGCCA TGAGCTGGAC CTCGTTGTGC CCGTCTTCAC GTTCCTGCAG |
| 841 | TTCTTCTTCT ATGTTGGCTG GCTGAAGGTG GCAGAGCAGC TCATCAACCC CTTTGGAGAG |
| 901 | GATGATGATG ATTTTGAGAC CAACTGGATT GTCGACAGGA ATTTGCAGGT GTCCCTGTTG |
| 961 | GCTGTGGATG AGATGCACCA GGACCTGCCT CGGATGGAGC CGGACATGTA CTGGAATAAG |
| 1021 | CCCGAGCCAC AGCCCCCCTA CACAGCTGCT TCCGCCCAGT TCCGTCGAGC CTCCTTTATG |
| 1081 | GGCTCCACCT TCAACATCAG CCTGAACAAA GAGGAGATGG AGTTCCAGCC CAATCAGGAG |
| 1141 | GACGAGGAGG ATGCTCACGC TGGCATCATT GGCCGCTTCC TAGGCCTGCA GTCCCATGAT |
| 1201 | CACCATCCTC CCAGGGCAAA CTCAAGGACC AAACTACTGT GGCCCAAGAG GGAATCCCTT |
| 1261 | CTCCACGAGG GCCTGCCCAA AAACCACAAG GCAGCCAAAC AGAACGTTAG GGGCCAGGAA |
| 1321 | GACAACAAGG CCTGGAAGCT TAAGGCTGTG GACGCCTTCA AGTCTGCCCC ACTGTATCAG |
| 1381 | AGGCCAGGCT ACTACAGTGC CCCACAGACG CCCCTCAGCC CCACTCCCAT GTTCTTCCCC |
| 1441 | CTAGAACCAT CAGCGCCGTC AAAGCTTCAC AGTGTCACAG GCATAGACAC CAAAGACAAA |
| 1501 | AGCTTAAAGA CTGTGAGTTC TGGGGCCAAG AAAAGTTTTG AATTGCTCTC AGAGAGCGAT |
| 1561 | GGGGCCTTGA TGGAGCACCC AGAAGTATCT CAAGTGAGGA GGAAAACTGT GGAGTTTAAC |
| 1621 | CTGACGGATA TGCCAGAGAT CCCCGAAAAT CACCTCAAAG AACCTTTGGA ACAATCACCA |
| 1681 | ACCAACATAC ACACTACACT CAAAGATCAC ATGGATCCTT ATTGGGCCTT GGAAAACAGG |
| 1741 | GATGAAGCAC ATTCCTAA. |

In some embodiments of the compositions of the disclosure, a nucleic acid sequence encoding a BEST1 protein of the disclosure comprises a codon optimized sequence. In some embodiments, the sequence has been codon optimized for expression in a mammalian cell. In some embodiments, the sequence has been codon optimized for expression in a human cell.

### BEST1 Expression

In some embodiments of the compositions of the disclosure, a nucleic acid sequence encoding a BEST1 protein of the disclosure further comprises a sequence encoding a regulatory element that enhances or increases BEST1 transcript or BEST1 protein expression. Exemplary regulatory element that enhances or increases BEST1 transcript or BEST1 protein expression include, but are not limited to, a promoter, an enhancer, a superenhancer, an intron, an exon, a combination of an intron and exon, a sequence encoding an untranslated region (e.g. a 5′ untranslated region (UTR) or a 3′ UTR), a sequence comprising a polyadenylation (polyA) signal, and a posttranscriptional regulatory element (PRE).

Exemplary promoters of the disclosure include, but are not limited to, those promoters capable of expressing a sequence encoding a BEST1 protein or a BEST1 protein in a mammalian cell. Exemplary promoters of the disclosure include, but are not limited to, those promoters capable of expressing a sequence encoding a BEST1 protein or a BEST1 protein in a human cell. In some embodiments, the mammalian or the human cell may be in vivo, ex vivo, in vitro or in situ. In some embodiments, the promoter may be constitutively active. In some embodiments, the promoter may be cell-type specific. In some embodiments, the promoter may be inducible.

Exemplary constitutively active promoters of the disclosure include, but are not limited to, a viral promoter. Viral promoters of the disclosure may include, but are not limited to, a simian virus 40 (SV40) promoter, a cytomegalovirus (CMV) promoter, ubiquitin C (UBC) promoter, elongation factor-1 alpha (EF1A) promoter, phosphoglycerate kinase 1 (PGK) promoter and a CAG promoter (a combination of a (C) the cytomegalovirus (CMV) early enhancer element, (A) the promoter comprising the first exon and the first intron of chicken beta-actin gene, and (G) the splice acceptor of the rabbit beta-globin gene). In some embodiments, a CMV promoter is used to control expression of a nucleic acid sequence encoding a BEST1 protein of the disclosure. In some embodiments, a CAG promoter is used to control expression of a nucleic acid sequence encoding a BEST1 protein of the disclosure. Non-viral promoters of the disclosure may include, but are not limited to, a chicken beta actin (CBA) promoter. In some embodiments, the CBA promoter comprises the chicken beta actin the first exon and intron of the CBA gene. In some embodiments, the promoter comprises the chicken beta actin promoter and the cytomegalovirus early enhancer elements. In some embodiments, the promoter further comprises a rabbit beta globin splice acceptor sequence (the CAG promoter). In some embodiments, the CAG promoter comprises or consists of a nucleic acid sequence having at least 80%, at least 90%, at least 95%, at least 96%, at least 97%, at least 98% or at least 99% identity to the nucleic acid sequence of:

| (SEQ ID NO: 2) | |
|---|---|
| 1 | CCATTGACGT CAATAATGAC GTATGTTCCC ATAGTAACGC CAATAGGGAC TTTCCATTGA |
| 61 | CGTCAATGGG TGGAGTATTT ACGGTAAACT GCCCACTTGG CAGTACATCA AGTGTATCAT |
| 121 | ATGCCAAGTA CGCCCCCTAT TGACGTCAAT GACGGTAAAT GGCCCGCCTG GCATTATGCC |
| 181 | CAGTACATGA CCTTATGGGA CTTTCCTACT TGGCAGTACA TCTACGTATT AGTCATCGCT |
| 241 | ATTACCATGG TCGAGGTGAG CCCCACGTTC TGCTTCACTC TCCCCATCTC CCCCCCCTCC |
| 301 | CCACCCCCAA TTTTGTATTT ATTTATTTTT TAATTATTTT GTGCAGCGAT GGGGGCGGGG |
| 361 | GGGGGGGGGG GGCGCGCGCC AGGCGGGGCG GGGCGGGGCG AGGGGCGGGG CGGGGCGAGG |
| 421 | CGGAGAGGTG CGGCGGCAGC CAATCAGAGC GGCGCGCTCC GAAAGTTTCC TTTTATGGCG |
| 481 | AGGCGGCGGC GGCGGCGGCC CTATAAAAAG CGAAGCGCGC GGCGGGCGGG AGTCGCTGCG |
| 541 | CGCTGCCTTC GCCCCGTGCC CCGCTCCGCC GCCGCCTCGC GCCGCCCGCC CCGGCTCTGA |
| 601 | CTGACCGCGT TACTCCCACA GGTGAGCGGG CGGGACGGCC CTTCTCCTCC GGGCTGTAAT |
| 661 | TAGCGCTTGG TTTAATGACG GCTTGTTTCT TTTCTGTGGC TGCGTGAAAG CCTTGAGGGG |
| 721 | CTCCGGGAGG GCCCTTTGTG CGGGGGGAGC GGCTCGGGGC TGTCCGCGGG GGGACGGCTG |
| 781 | CCTTCGGGGG GGACGGGGCA GGGCGGGGTT CGGCTTCTGG CGTGTGACCG GCGGCTCTAG |
| 841 | AGCCTCTGCT AACCATGTTC ATGCCTTCTT CTTTTTCCTA CAGCTCCTGG GCAACGTGCT |
| 901 | GGTTATTGTG CTGTCTCATC ATTTTGGCAA AGAATTGGAT C. |

Exemplary cell-type specific promoters of the disclosure include, but are not limited to, a promoter capable of expressing a nucleic acid or a protein in a neuron, a promoter capable of expressing a nucleic acid or a protein in a retinal cell, a promoter capable of expressing a nucleic acid or a protein in a photoreceptor, a promoter capable of expressing a nucleic acid or a protein in a rod cell, and a promoter capable of expressing a nucleic acid or a protein in a cone cell. In some embodiments, a sequence encoding a tissue specific promoter comprises a sequence encoding a human VMD2 gene (also known as Bestrophin-1). In some embodiments, a tissue specific promoter comprises a human VMD2 promoter (also known as Bestrophin-1). In some embodiments, the human VMD2 promoter comprises or consists of a nucleic acid sequence having at least 80%, at least 90%, at least 95%, at least 96%, at least 97%, at least 98%, at least 99% identity to the nucleic acid sequence of:

| (SEQ ID NO: 1) | |
|---|---|
| 1 | AATTCTGTCA TTTTACTAGG GTGATGAAAT TCCCAAGCAA CACCATCCTT TTCAGATAAG |
| 61 | GGCACTGAGG CTGAGAGAGG AGCTGAAACC TACCCGGGGT CACCACACAC AGGTGGCAAG |
| 121 | GCTGGGACCA GAAACCAGGA CTGTTGACTG CAGCCCGGTA TTCATTCTTT CCATAGCCCA |
| 181 | CAGGGCTGTC AAAGACCCCA GGGCCTAGTC AGAGGCTCCT CCTTCCTGGA GAGTTCCTGG |
| 241 | CACAGAAGTT GAAGCTCAGC ACAGCCCCCT AACCCCCAAC TCTCTCTGCA AGGCCTCAGG |
| 301 | GGTCAGAACA CTGGTGGAGC AGATCCTTTA GCCTCTGGAT TTTAGGGCCA TGGTAGAGGG |
| 361 | GGTGTTGCCC TAAATTCCAG CCCTGGTCTC AGCCCAACAC CCTCCAAGAA GAAATTAGAG |
| 421 | GGGCCATGGC CAGGCTGTGC TAGCCGTTGC TTCTGAGCAG ATTACAAGAA GGGACTAAGA |
| 481 | CAAGGACTCC TTTGTGGAGG TCCTGGCTTA GGGAGTCAAG TGACGGCGGC TCAGCACTCA |
| 541 | CGTGGGCAGT GCCAGCCTCT AAGAGTGGGC AGGGGCACTG GCCACAGAGT CCCAGGGAGT |
| 601 | CCCACCAGCC TAGTCGCCAG ACC. |

In some embodiments, the human VMD2 promoter comprises or consists of a nucleic acid sequence having 100% identity to the nucleic acid sequence of:

| (SEQ ID NO: 1) | |
|---|---|
| 1 | AATTCTGTCA TTTTACTAGG GTGATGAAAT TCCCAAGCAA CACCATCCTT TTCAGATAAG |
| 61 | GGCACTGAGG CTGAGAGAGG AGCTGAAACC TACCCGGGGT CACCACACAC AGGTGGCAAG |
| 121 | GCTGGGACCA GAAACCAGGA CTGTTGACTG CAGCCCGGTA TTCATTCTTT CCATAGCCCA |
| 181 | CAGGGCTGTC AAAGACCCCA GGGCCTAGTC AGAGGCTCCT CCTTCCTGGA GAGTTCCTGG |
| 241 | CACAGAAGTT GAAGCTCAGC ACAGCCCCCT AACCCCCAAC TCTCTCTGCA AGGCCTCAGG |
| 301 | GGTCAGAACA CTGGTGGAGC AGATCCTTTA GCCTCTGGAT TTTAGGGCCA TGGTAGAGGG |
| 361 | GGTGTTGCCC TAAATTCCAG CCCTGGTCTC AGCCCAACAC CCTCCAAGAA GAAATTAGAG |
| 421 | GGGCCATGGC CAGGCTGTGC TAGCCGTTGC TTCTGAGCAG ATTACAAGAA GGGACTAAGA |
| 481 | CAAGGACTCC TTTGTGGAGG TCCTGGCTTA GGGAGTCAAG TGACGGCGGC TCAGCACTCA |
| 541 | CGTGGGCAGT GCCAGCCTCT AAGAGTGGGC AGGGGCACTG GCCACAGAGT CCCAGGGAGT |
| 601 | CCCACCAGCC TAGTCGCCAG ACC. |

In some embodiments of the compositions of the disclosure, the nucleic acid sequence comprising a sequence encoding a BEST1 protein and a sequence encoding a promoter, further comprises an intron and an exon. The presence of an intron and an exon increases levels of protein expression. In some embodiments, the intron is positioned between the VMD2 promoter and the exon. In some embodiments, including those embodiments wherein the intron is positioned between the VMD2 promoter and the exon, the exon is positioned 5′ of the BEST coding sequence.

The exon may comprise a coding sequence, a non-coding sequence, or a combination of both. In some embodiments, the exon comprises non-coding sequence. In some embodiments, the exon is isolated or derived from a mammalian gene. In embodiments, the mammal is a rabbit (Oryctolagus cuniculus). In some embodiments, the mammalian gene comprises a rabbit beta globin gene. In some embodiments, the exon comprises a nucleic acid sequence having at least 80%, at least 90%, at least 95%, at least 96%, at least 97%, at least 98%, or at least 99% identity to the nucleic acid sequence of:

(SEQ ID NO: 6)

CTCCTGGGCA ACGTGCTGGT TATTGTGCTG TCTCATCATT
TTGGCAAAGA ATT.

In some embodiments, the exon comprises a nucleic acid sequence having 100% identify to the nucleic acid sequence of:

(SEQ ID NO: 6)

CTCCTGGGCA ACGTGCTGGT TATTGTGCTG TCTCATCATT
TTGGCAAAGA ATT.

Introns may comprise a splice donor site, a splice acceptor site or a branch point. Introns may comprise a splice donor site, a splice acceptor site and a branch point. Exemplary splice acceptor sites comprise nucleotides “GT” (“GU” in the pre-mRNA) at the 5′ end of the intron. Exemplary splice acceptor sites comprise an “AG” at the 3′ end of the intron. In some embodiments, the branch point comprises an adenosine (A) between 20 and 40 nucleotides, inclusive of the endpoints, upstream of the 3′ end of the intron. The intron may be an artificial or non-naturally occurring sequence. Alternatively, the intron may be isolated or derived from a vertebrate gene. The intron may comprise a sequence encoding a fusion of two sequences, each of which may be isolated or derived from a plurality of vertebrate genes. In some embodiments, a vertebrate gene contributing to the intron nucleic acid sequence comprises a chicken (Gallus gallus) gene. In some embodiments, the chicken gene comprises the chicken beta actin gene. In some embodiments, a vertebrate gene contributing to the intron nucleic acid sequence comprises a rabbit (Oryctolagus cuniculus) gene. In some embodiments, the rabbit gene comprises the rabbit beta globin gene. In some embodiments, the intron comprises a nucleic acid sequence having at least 80%, at least 90%, at least 95%, at least 96%, at least 97%, at least 98%, or at least 99% identity to the nucleic acid sequence of:

| (SEQ ID NO: 7) | |
|---|---|
| 1 | GTGCCGCAGG GGGACGGCTG CCTTCGGGGG GGACGGGGCA GGGCGGGGTT |
| | CGGCTTCTGG |
| 61 | CGTGTGACCG GCGGCTCTAG AGCCTCTGCT AACCATGTTC ATGCCTTCTT |
| | CTTTTTCCTA |
| 121 | CAG. |

In some embodiments, the intron comprises a nucleic acid sequence having 100% identify to the nucleic acid sequence of:

| (SEQ ID NO: 7) | |
|---|---|
| 1 | GTGCCGCAGG GGGACGGCTG CCTTCGGGGG GGACGGGGCA GGGCGGGGTT |
| | CGGCTTCTGG |
| 61 | CGTGTGACCG GCGGCTCTAG AGCCTCTGCT AACCATGTTC ATGCCTTCTT |
| | CTTTTTCCTA |
| 121 | CAG. |

Kozak sequences are short sequence motifs that are recognized by the ribosome as the translation start site. Kozak sequences may be positioned immediately upstream, or surrounding the translational start site. In vertebrates, the Kozak consensus sequence comprises a sequence of having at least 50% identity to the consensus sequence of gccRccATGG, where R represents an A or G, and the ATG encoding the start methionine is bolded. An exemplary Kozak sequence of the disclosure comprises a sequence of GGCACCATGA. In some embodiments, the nucleic acid comprising a nucleic acid sequence encoding BEST1, further comprises a sequence encoding a 5′ untranslated sequence (5′ UTR). In some embodiments, the 5′ UTR comprises a Kozak sequence. In some embodiments, the 5′ UTR comprises a portion of a Kozak sequence. In some embodiments, the 5′ UTR comprises at least 50%, at least 60%, at least 70% or at least 80% of a Kozak sequence.

In some embodiments, the nucleic acid comprising a nucleic acid sequence encoding BEST1, further comprises a nucleic acid sequence encoding transcriptional response element (PRE). Exemplary PREs comprise a Woodchuck PRE (WPRE), which is derived from the Woodchuck hepatitis virus. In some embodiments, a sequence encoding a WPRE is positioned 3′ of the nucleic acid sequence encoding BEST1. In some embodiments, a sequence encoding a WPRE is positioned between the nucleic acid sequence encoding BEST1 and the sequence encoding a polyA signal. In some embodiments, a sequence encoding a WPRE comprises a nucleic acid sequence having at least 80%, at least 90%, at least 95%, at least 96%, at least 97%, at least 98%, or at least 99% identity to the nucleic acid sequence of:

| (SEQ ID NO: 8) | |
|---|---|
| 1 | ATCGATAATC AACCTCTGGA TTACAAAATT TGTGAAAGAT TGACTGGTAT TCTTAACTAT |
| 61 | GTTGCTCCTT TTACGCTATG TGGATACGCT GCTTTAATGC CTTTGTATCA TGCTATTGCT |
| 121 | TCCCGTATGG CTTTCATTTT CTCCTCCTTG TATAAATCCT GGTTGCTGTC TCTTTATGAG |
| 181 | GAGTTGTGGC CCGTTGTCAG GCAACGTGGC GTGGTGTGCA CTGTGTTTGC TGACGCAACC |
| 241 | CCCACTGGTT GGGGCATTGC CACCACCTGT CAGCTCCTTT CCGGGACTTT CGCTTTCCCC |
| 301 | CTCCCTATTG CCACGGCGGA ACTCATCGCC GCCTGCCTTG CCCGCTGCTG GACAGGGGCT |
| 361 | CGGCTGTTGG GCACTGACAA TTCCGTGGTG TTGTCGGGGA AATCATCGTC CTTTCCTTGG |
| 421 | CTGCTCGCCT GTGTTGCCAC CTGGATTCTG CGCGGGACGT CCTTCTGCTA CGTCCCTTCG |
| 481 | GCCCTCAATC CAGCGGACCT TCCTTCCCGC GGCCTGCTGC CGGCTCTGCG GCCTCTTCCG |
| 541 | CGTCTTCGCC TTCGCCCTCA GACGAGTCGG ATCTCCCTTT GGGCCGCCTC CCC. |

In some embodiments, a sequence encoding a WPRE comprises a nucleic acid sequence having 100% identity to the nucleic acid sequence of:

| (SEQ ID NO: 8) | |
|---|---|
| 1 | ATCGATAATC AACCTCTGGA TTACAAAATT TGTGAAAGAT TGACTGGTAT TCTTAACTAT |
| 61 | GTTGCTCCTT TTACGCTATG TGGATACGCT GCTTTAATGC CTTTGTATCA TGCTATTGCT |
| 121 | TCCCGTATGG CTTTCATTTT CTCCTCCTTG TATAAATCCT GGTTGCTGTC TCTTTATGAG |
| 181 | GAGTTGTGGC CCGTTGTCAG GCAACGTGGC GTGGTGTGCA CTGTGTTTGC TGACGCAACC |
| 241 | CCCACTGGTT GGGGCATTGC CACCACCTGT CAGCTCCTTT CCGGGACTTT CGCTTTCCCC |
| 301 | CTCCCTATTG CCACGGCGGA ACTCATCGCC GCCTGCCTTG CCCGCTGCTG GACAGGGGCT |
| 361 | CGGCTGTTGG GCACTGACAA TTCCGTGGTG TTGTCGGGGA AATCATCGTC CTTTCCTTGG |
| 421 | CTGCTCGCCT GTGTTGCCAC CTGGATTCTG CGCGGGACGT CCTTCTGCTA CGTCCCTTCG |
| 481 | GCCCTCAATC CAGCGGACCT TCCTTCCCGC GGCCTGCTGC CGGCTCTGCG GCCTCTTCCG |
| 541 | CGTCTTCGCC TTCGCCCTCA GACGAGTCGG ATCTCCCTTT GGGCCGCCTC CCC. |

In some embodiments, the nucleic acid comprising a nucleic acid sequence encoding BEST1, further comprises a sequence encoding a polyadenylation (polyA) signal. The polyA signal facilitates nuclear export, enhances translation and increases mRNA stability. In some embodiments, the sequence encoding the polyA signal comprises a synthetic or an artificial sequence. In some embodiments, the sequence encoding the polyA signal comprises a sequence isolated or derived from a mammalian gene. In some embodiments, the mammalian gene is a human gene. In some embodiments, the mammalian gene is a bovine growth hormone gene (BGH). In some embodiments, the sequence encoding the polyA signal comprises a nucleic acid sequence having at least 80%, at least 90%, at least 95%, at least 96%, at least 97%, at least 98%, at least 99% identity to the nucleic acid sequence of:

| (SEQ ID NO: 9) | |
|---|---|
| 1 | CGCTGATCAG CCTCGACTGT GCCTTCTAGT TGCCAGCCAT CTGTTGTTTG CCCCTCCCCC |
| 61 | GTGCCTTCCT TGACCCTGGA AGGTGCCACT CCCACTGTCC TTTCCTAATA AAATGAGGAA |
| 121 | ATTGCATCGC ATTGTCTGAG TAGGTGTCAT TCTATTCTGG GGGGTGGGGT GGGGCAGGAC |
| 181 | AGCAAGGGGG AGGATTGGGA AGACAATAGC AGGCATGCTG GGGATGCGGT GGGCTCTATG |
| 241 | GCTTCTGAGG CGGAAAGAAC CAGCTGGGG. |

In some embodiments, the sequence encoding the polyA signal comprises a nucleic acid sequence having 100% identity to the nucleic acid sequence of:

| (SEQ ID NO: 9) | |
|---|---|
| 1 | CGCTGATCAG CCTCGACTGT GCCTTCTAGT TGCCAGCCAT CTGTTGTTTG CCCCTCCCCC |
| 61 | GTGCCTTCCT TGACCCTGGA AGGTGCCACT CCCACTGTCC TTTCCTAATA AAATGAGGAA |
| 121 | ATTGCATCGC ATTGTCTGAG TAGGTGTCAT TCTATTCTGG GGGGTGGGGT GGGGCAGGAC |
| 181 | AGCAAGGGGG AGGATTGGGA AGACAATAGC AGGCATGCTG GGGATGCGGT GGGCTCTATG |
| 241 | GCTTCTGAGG CGGAAAGAAC CAGCTGGGG. |

### AAV Vectors

A vector may comprise the nucleic acid comprising a nucleic acid sequence encoding BEST1. In some embodiments of the compositions of the disclosure, the vector may be a viral delivery vector. Viral delivery vectors of the disclosure may contain sequences necessary for packaging a nucleic acid sequence of the disclosure into a viral delivery system for delivery to a target cell or tissue. Typical viral delivery vectors of the disclosure include, but are not limited to, lentiviral, retroviral or adeno-associated viral (AAV) vectors.

An AAV viral delivery system of the disclosure may be in the form of a mature AAV particle or virion, i.e. nucleic acid surrounded by an AAV protein capsid. In some embodiments, the AAV viral delivery vector may comprise an AAV genome or a derivative thereof.

An AAV genome is a nucleic acid sequence which encodes functions needed for production of an AAV particle. These functions include those operating in the replication and packaging cycle of AAV in a host cell, including encapsidation of the AAV genome into an AAV particle. Naturally occurring AAVs are replication-deficient and rely on the provision of helper functions in trans for completion of a replication and packaging cycle. In preferred embodiments, an AAV genome of a vector of the disclosure is replication-deficient.

The AAV genome may be in single-stranded form, either positive or negative-sense, or alternatively in double-stranded form. The use of a double-stranded form allows bypass of the DNA replication step in the target cell and so can accelerate transgene expression. The AAV genome of a vector of the disclosure may be single-stranded form.

The AAV genome may be from any naturally derived serotype, isolate or clade of AAV. Thus, the AAV genome may be the full genome of a naturally occurring AAV. As is known to the person skilled in the art, AAVs occurring in nature may be classified according to various biological systems.

AAVs are referred to in terms of their serotype. A serotype corresponds to a variant subspecies of AAV which, owing to its profile of expression of capsid surface antigens, has a distinctive reactivity which can be used to distinguish it from other variant subspecies. A virus having a particular AAV serotype does not efficiently cross-react with neutralizing antibodies specific for any other AAV serotype. AAV serotypes include AAV1, AAV2, AAV3, AAV4, AAV5, AAV6, AAV7, AAV8, AAV9, AAV10 and AAV11, and also recombinant serotypes, such as Rec2 and Rec3, recently identified from primate brain. Any of these AAV serotypes may be used in the invention. Thus, in some embodiments, an AAV vector of the invention may be derived from an AAV1, AAV2, AAV3, AAV4, AAV5, AAV6, AAV7, AAV8, AAV9, AAV10, AAV11, Rec2 or Rec3 AAV.

Reviews of AAV serotypes may be found in Choi et al. (2005) Cur. Gene There. 5: 299-310 and Wu et al. (2006) Molecular Therapy 14: 316-27. The sequences of AAV genomes or of elements of AAV genomes including ITR sequences, rep or cap genes may be derived from the following accession numbers for AAV whole genome sequences: Adeno-associated virus 1 NC_002077, AF063497; Adeno-associated virus 2 NC_001401; Adeno-associated virus 3 NC_001729; Adeno-associated virus 3B NC_001863; Adeno-associated virus 4 NC_001829; Adeno-associated virus 5 Y18065, AF085716; Adeno-associated virus 6 NC_001862; Avian AAV ATCC VR-865 AY186198, AY629583, NC_004828; Avian AAV strain DA-1 NC_006263, AY629583; Bovine AAV NC_005889, AY388617.

AAV may also be referred to in terms of clades or clones. This refers to the phylogenetic relationship of naturally derived AAVs, as well as to a phylogenetic group of AAVs which can be traced back to a common ancestor, and includes all descendants thereof.

Additionally, AAVs may be referred to in terms of a specific isolate, i.e. a genetic isolate of a specific AAV found in nature. The term genetic isolate describes a population of AAVs which has undergone limited genetic mixing with other naturally occurring AAVs, thereby defining a recognizably distinct population at a genetic level.

The AAV serotype determines the tissue specificity of infection (or tropism) of an AAV virus. Accordingly, preferred AAV serotypes for use in AAVs administered to patients in accordance with the invention are those which have natural tropism for or a high efficiency of infection of target cells within the eye. In one embodiment, AAV serotypes for use in the invention are those which infect cells of the neurosensory retina, retinal pigment epithelium and/or macula.

The AAV genome of a naturally derived serotype, isolate or clade of AAV comprises at least one inverted terminal repeat sequence (ITR). An ITR sequence acts in cis to provide a functional origin of replication and allows for integration and excision of the vector from the genome of a cell.

An AAV viral delivery vector may include at least one inverted terminal repeat sequence (ITR), preferably more than one ITR, such as two ITRs or more. One or more of the ITRs may be derived from AAV genomes having different serotypes, or may be a chimeric or mutant ITR. A preferred mutant ITR is one having a deletion of a trs (terminal resolution site). This deletion allows for continued replication of the genome to generate a single-stranded genome which contains both coding and complementary sequences, i.e. a self-complementary AAV genome. This allows for bypass of DNA replication in the target cell, and so enables accelerated transgene expression.

The inclusion of one or more ITRs is preferred to aid concatamer formation of a viral delivery vector of the invention in the nucleus of a host cell, for example following the conversion of single-stranded vector DNA into double-stranded DNA by the action of host cell DNA polymerases. The formation of such episomal concatamers protects the vector construct during the life of the host cell, thereby allowing for prolonged expression of the transgene in vivo.

In some embodiments, ITR elements are the only sequences retained from the native AAV genome in the viral delivery vector. Thus, in some embodiments, a viral delivery vector does not include either the rep or cap genes of the native genome and, furthermore, lacks any other sequences of the native genome. This is preferred for the reasons described above, and also to reduce the possibility of integration of the vector into the host cell genome.

Additionally, reducing the size of the AAV genome allows for increased flexibility in incorporating other sequence elements (such as regulatory elements) within the vector in addition to the transgene. In some embodiments, the viral delivery vector of the disclosure comprises sequences encoding AAV2 ITRs. In some embodiments, the sequences encoding the two AAV2 ITRs may comprise or consist of a nucleic acid sequence of:

| (SEQ ID NO: 10) | |
|---|---|
| 1 | ctgcgcgctc gctcgctcac tgaggccgcc cgggcaaagc ccgggcgtcg ggcgaccttt |
| 61 | ggtcgcccgg cctcagtgag cgagcgagcg cgcagagagg gagtggccaa ctccatcact |
| 121 | aggggttcct tgtagttaat gatt. |

and/or

| (SEQ ID NO: 11) | |
|---|---|
| 1 | tgcgcgctcg ctcgctcact gaggccgggc gaccaaaggt cgcccgacgc ccgggctttg |
| 61 | cccgggcggc ctcagtgagc gagcgagcgc gcagagcttt ttgcaaaagc ctaggcctcc |
| 121 | aaaaaagcct cctcactact tctgg. |

The AAV genome may comprise a nucleic acid sequence of about 4.7 kb in length. Thus, in those embodiments where the nucleic acid sequence to be delivered by an AAV viral vector is less than 4.7 kb in length, a stuffer or filler sequence may be used. The presence of a stuffer sequence can, in some embodiments, aid in AAV viral vector packaging into the viral particle. In some embodiments, the stuffer sequence comprises a random sequence. An exemplary stuffer sequence of the disclosure may comprise or consist of the nucleic acid sequence of:

| (SEQ ID NO: 12) | |
|---|---|
| 1 | GATGTAACCA TATACTTAGG CTGGATCTTC TCCCGCGAAT TTTAACCCTC ACCAACTACG |
| 61 | AGATATGAGG TAAGCCAAAA AAGCACGTAG TGGCGCTCTC CGACTGTTCC CAAATTGTAA |
| 121 | CTTATCGTTC CGTGAAGGCC AGAGTTACTT CCCGGCCCTT TCCATGCGCG CACCATACCC |
| 181 | TCCTAGTTCC CCGGTTATCT TTCCGAAGTG GGAGTGAGCG AACCTCCGTT TACGTCTTGT |
| 241 | TACCAATGAT GTAGCTATGC ACTTTGTACA GGGTGCCAAC GGGTTTCACA ATTCACAGAT |
| 301 | AGTGGGGATC CCGGCAAAGG GCCTATATTT GCGGTCCAAC TTAGGCGTAA ACCTCGATGC |
| 361 | TACCTACTCA GACCCACCTC GCGCGGGGTA AATAAGGCAC TCATCCCAGC TGGTTCTTGG |
| 421 | CGTTCTACGC AGCGACATGT TTATTAACAG TTGTCTGGCA GCACAAAACT TTTACCATGG |
| 481 | TCGTAGAAGC CCCCCAGAGT TAGTTCATAC CTAATGCCAC AAATGTGACA GGACGCCGAT |
| 541 | GGGTACCGGA CTTTAGGTCG AGCACAGTTC GGTAACGGAG AGACCCTGCG GCGTACTTCA |
| 601 | TTATGTATAT GGAACGTGCC CAAGTGACGC CAGGCAAGTC TCAGCTGGTT CCTGTGTTAG |
| 661 | CTCGAGGGTA GACATACGAG CTGATTGAAC ATGGGTTGGG GGCCTCGAAC CGTCGAGGAC |
| 721 | CCCATAGTAC CTCGGAGACC AAGTAGGGCA GCCTATAGTT TGAAGCAGAA CTATTTCGGG |
| 781 | GGGCGAGCCC TCATCGTCTC TTCTGCGGAT GACTCAACAC GCTAGGGACG TGAAGTCGAT |
| 841 | TCCTTCGATG GTTATAAATC AAAGACTCAG AGTGCTGTCT GGAGCGTGAA TCTAACGGTA |
| 901 | CGTATCTCGA TTGCTCGGTC GCTTTTCGCA CTCCGCGAAA GTTCGTACCG CTCATTCACT |
| 961 | AGGTTGCGAA GCCTATGCTG ATATATGAAT CCAAACTAGA GCAGGGCTCT TAAGATTCGG |
| 1021 | AGTTGTAAAT ACTTAATACT CCAATCGGCT TTTACGTGCA CCACCGCGGG CGGCTGACAA |
| 1081 | GGGTCTCACA TCGAGAAACA AGACAGTTCC GGGCTGGAAG TAGCGCCGGC TAAGGAAGAC |
| 1141 | GCCTGGTACG GCAGGACTAT GAAACCAGTA CAAAGGCAAC ATCCTCACTT GGGTGAACGG |
| 1201 | AAACGCAGTA TTATGGTTAC TTTTTGGATA CGTGAAACAT ATCCCATGGT AGTCCTTAGA |
| 1261 | CTTGGGAGTC TATCACCCCT AGGGCCCATA TCTGGAAATA GACGCCAGGT TGAATCCGTA |
| 1321 | TTTGGAGGTA CGATGGAACA GTCTGGGTGG GACGTGCTTC ATTTATACCC TGCGCAGGCT |
| 1381 | GGACCGAGGA CCGCAAGGTG CGGCGGTGCA CAAGCAATTG ACAACTAACC ACCGTGTATT |
| 1441 | CATTATGGTA CCAGGAACTT TAAGCCGAGT CAATGAAGCT CGCATTACAG TGTTTACCGC |
| 1501 | ATCTTGCCGT TACTCACAAA CTGTGATCCA CCACAAGTCA AGCCATTGCC TCTCTGACAC |
| 1561 | GCCGTAAGAA TTAATATGTA AACTTTGCGC GGGTTGACTG CGATCCGTTC AGTCTCGTCC |
| 1621 | GAGGGCACAA TCCTATTCCC ATTTGTATGT TCAGCTAACT TCTACCCATC CCCCGAAGTT |
| 1681 | AAGTAGGTCG TGAGATGCCA TGGAGGCTCT CGTTCATCCC GTGGGACATC AAGCTTCCCC |
| 1741 | TTGATAAAGC ACCCCGCTCG GGTGTAGCAG AGAAGACGCC TTCTGAATTG TGCAATCCCT |
| 1801 | CCACCTTATC TAAGCTTGCT ACCAATAATT AGCATTTTTG CCTTGCGACA GACCTCCTAC |
| 1861 | TTAGATTGCC ACACATTGAG CTAGTCAGTG AGCGATAAGC TTGACGCGCT TTCAAGGGTC |
| 1921 | GCGAGTACGT GAACTAAGGC TCCGGACAGG ACTATATACT TGGGTTTGAT CTCGCCCCGA |
| 1981 | CAACTGCAAA CCTCAACTTT TTTAGATTAT ATGGTTAGCC GAAGTTGCAC GAGGTGGCGT |
| 2041 | CCGCGGACTG CTCCCCGAGT GTGGCTCTTT CATCTGACAA CGTGCAACCC CTATCGCGGC |
| 2101 | CGATTGTTTC TGCGGACGAT GTTGTCCTCA TAGTTTGGGC ATGTTTCCCT TGTAGGTGTG |
| 2161 | AAACCACTTA GCTTCGCGCC GTAGTCCCAA TGAAAAACCT ATGGACTTTG TTTTGGGTAG |
| 2221 | CACCAGGAAT CTGAACCGTG TGAATGTGGA CGTCGCGCGC GTAGACCTTT ATCTCCGGTT |
| 2281 | CAAGCTAGGG ATGTGGCTGC ATGCTACGTT GTCACACCTA CACTGCTCGA AGTAAATATG |
| 2341 | CGAAGCGCGC GGCCTGGCCG GAGGCGTTCC GCGCCGCCAC GTGTTCGTTA ACTGTTGATT |
| 2401 | GGTGGCACAT AAGCAATATC GTAGTCCGTC AAATTCAGCT CTGTTATCCC GGGCGTTATG |
| 2461 | TGTCAAATGG CGTAGAACGG GATTGACTGT TTGACGGTAG. |

In some embodiments, the AAV viral delivery vector comprises a nucleic acid sequence comprising a sequence encoding a VMD2 promoter, a sequence encoding a BEST1 protein, and a sequence encoding a WPRE. An exemplary AAV viral delivery vector of the disclosure comprising this nucleic acid sequence (VMD2.BEST1.WPRE.pA) comprises or consists of the nucleic acid sequence of:

| (SEQ ID NO: 13) | |
|---|---|
| 1 | TAGCTGCGCG CTCGCTCGCT CACTGAGGCC GCCCGGGCAA AGCCCGGGCG TCGGGCGACC |
| 61 | TTTGGTCGCC CGGCCTCAGT GAGCGAGCGA GCGCGCAGAG AGGGAGTGGC CAACTCCATC |
| 121 | ACTAGGGGTT CCTTGTAGTT AATGATTAAC CCGCCATGCT ACTTATCTAC GTAGCCATGC |
| 181 | TCTAGGTAAA TTCTGTCATT TTACTAGGGT GATGAAATTC CCAAGCAACA CCATCCTTTT |
| 241 | CAGATAAGGG CACTGAGGCT GAGAGAGGAG CTGAAACCTA CCCGGGGTCA CCACACACAG |
| 301 | GTGGCAAGGC TGGGACCAGA AACCAGGACT GTTGACTGCA GCCCGGTATT CATTCTTTCC |
| 361 | ATAGCCCACA GGGCTGTCAA AGACCCCAGG GCCTAGTCAG AGGCTCCTCC TTCCTGGAGA |
| 421 | GTTCCTGGCA CAGAAGTTGA AGCTCAGCAC AGCCCCCTAA CCCCCAACTC TCTCTGCAAG |
| 481 | GCCTCAGGGG TCAGAACACT GGTGGAGCAG ATCCTTTAGC CTCTGGATTT TAGGGCCATG |
| 541 | GTAGAGGGGG TGTTGCCCTA AATTCCAGCC CTGGTCTCAG CCCAACACCC TCCAAGAAGA |
| 601 | AATTAGAGGG GCCATGGCCA GGCTGTGCTA GCCGTTGCTT CTGAGCAGAT TACAAGAAGG |
| 661 | GACTAAGACA AGGACTCCTT TGTGGAGGTC CTGGCTTAGG GAGTCAAGTG ACGGCGGCTC |
| 721 | AGCACTCACG TGGGCAGTGC CAGCCTCTAA GAGTGGGCAG GGGCACTGGC CACAGAGTCC |
| 781 | CAGGGAGTCC CACCAGCCTA GTCGCCAGAC CGGCACCATG ACCATCACTT ACACAAGCCA |
| 841 | AGTGGCTAAT GCCCGCTTAG GCTCCTTCTC CCGCCTGCTG CTGTGCTGGC GGGGCAGCAT |
| 901 | CTACAAGCTG CTATATGGCG AGTTCTTAAT CTTCCTGCTC TGCTACTACA TCATCCGCTT |
| 961 | TATTTATAGG CTGGCCCTCA CGGAAGAACA ACAGCTGATG TTTGAGAAAC TGACTCTGTA |
| 1021 | TTGCGACAGC TACATCCAGC TCATCCCCAT TTCCTTCGTG CTGGGCTTCT ACGTGACGCT |
| 1081 | GGTCGTGACC CGCTGGTGGA ACCAGTACGA GAACCTGCCG TGGCCCGACC GCCTCATGAG |
| 1141 | CCTGGTGTCG GGCTTCGTCG AAGGCAAGGA CGAGCAAGGC CGGCTGCTGC GGCGCACGCT |
| 1201 | CATCCGCTAC GCCAACCTGG GCAACGTGCT CATCCTGCGC AGCGTCAGCA CCGCAGTCTA |
| 1261 | CAAGCGCTTC CCCAGCGCCC AGCACCTGGT GCAAGCAGGC TTTATGACTC CGGCAGAACA |
| 1321 | CAAGCAGTTG GAGAAACTGA GCCTACCACA CAACATGTTC TGGGTGCCCT GGGTGTGGTT |
| 1381 | TGCCAACCTG TCAATGAAGG CGTGGCTTGG AGGTCGAATC CGGGACCCTA TCCTGCTCCA |
| 1441 | GAGCCTGCTG AACGAGATGA ACACCTTGCG TACTCAGTGT GGACACCTGT ATGCCTACGA |
| 1501 | CTGGATTAGT ATCCCACTGG TGTATACACA GGTGGTGACT GTGGCGGTGT ACAGCTTCTT |
| 1561 | CCTGACTTGT CTAGTTGGGC GGCAGTTTCT GAACCCAGCC AAGGCCTACC CTGGCCATGA |
| 1621 | GCTGGACCTC GTTGTGCCCG TCTTCACGTT CCTGCAGTTC TTCTTCTATG TTGGCTGGCT |
| 1681 | GAAGGTGGCA GAGCAGCTCA TCAACCCCTT TGGAGAGGAT GATGATGATT TTGAGACCAA |
| 1741 | CTGGATTGTC GACAGGAATT TGCAGGTGTC CCTGTTGGCT GTGGATGAGA TGCACCAGGA |
| 1801 | CCTGCCTCGG ATGGAGCCGG ACATGTACTG GAATAAGCCC GAGCCACAGC CCCCCTACAC |
| 1861 | AGCTGCTTCC GCCCAGTTCC GTCGAGCCTC CTTTATGGGC TCCACCTTCA ACATCAGCCT |
| 1921 | GAACAAAGAG GAGATGGAGT TCCAGCCCAA TCAGGAGGAC GAGGAGGATG CTCACGCTGG |
| 1981 | CATCATTGGC CGCTTCCTAG GCCTGCAGTC CCATGATCAC CATCCTCCCA GGGCAAACTC |
| 2041 | AAGGACCAAA CTACTGTGGC CCAAGAGGGA ATCCCTTCTC CACGAGGGCC TGCCCAAAAA |
| 2101 | CCACAAGGCA GCCAAACAGA ACGTTAGGGG CCAGGAAGAC AACAAGGCCT GGAAGCTTAA |
| 2161 | GGCTGTGGAC GCCTTCAAGT CTGCCCCACT GTATCAGAGG CCAGGCTACT ACAGTGCCCC |
| 2221 | ACAGACGCCC CTCAGCCCCA CTCCCATGTT CTTCCCCCTA GAACCATCAG CGCCGTCAAA |
| 2281 | GCTTCACAGT GTCACAGGCA TAGACACCAA AGACAAAAGC TTAAAGACTG TGAGTTCTGG |
| 2341 | GGCCAAGAAA AGTTTTGAAT TGCTCTCAGA GAGCGATGGG GCCTTGATGG AGCACCCAGA |
| 2401 | AGTATCTCAA GTGAGGAGGA AAACTGTGGA GTTTAACCTG ACGGATATGC CAGAGATCCC |
| 2461 | CGAAAATCAC CTCAAAGAAC CTTTGGAACA ATCACCAACC AACATACACA CTACACTCAA |
| 2521 | AGATCACATG GATCCTTATT GGGCCTTGGA AAACAGGGAT GAAGCACATT CCTAATCTAG |
| 2581 | CGGCCGCGAA TTCGATATCA AGCTTATCGA TAATCAACCT CTGGATTACA AAATTTGTGA |
| 2641 | AAGATTGACT GGTATTCTTA ACTATGTTGC TCCTTTTACG CTATGTGGAT ACGCTGCTTT |
| 2701 | AATGCCTTTG TATCATGCTA TTGCTTCCCG TATGGCTTTC ATTTTCTCCT CCTTGTATAA |
| 2761 | ATCCTGGTTG CTGTCTCTTT ATGAGGAGTT GTGGCCCGTT GTCAGGCAAC GTGGCGTGGT |
| 2821 | GTGCACTGTG TTTGCTGACG CAACCCCCAC TGGTTGGGGC ATTGCCACCA CCTGTCAGCT |
| 2881 | CCTTTCCGGG ACTTTCGCTT TCCCCCTCCC TATTGCCACG GCGGAACTCA TCGCCGCCTG |
| 2941 | CCTTGCCCGC TGCTGGACAG GGGCTCGGCT GTTGGGCACT GACAATTCCG TGGTGTTGTC |
| 3001 | GGGGAAATCA TCGTCCTTTC CTTGGCTGCT CGCCTGTGTT GCCACCTGGA TTCTGCGCGG |
| 3061 | GACGTCCTTC TGCTACGTCC CTTCGGCCCT CAATCCAGCG GACCTTCCTT CCCGCGGCCT |
| 3121 | GCTGCCGGCT CTGCGGCCTC TTCCGCGTCT TCGCCTTCGC CCTCAGACGA GTCGGATCTC |
| 3181 | CCTTTGGGCC GCCTCCCCGG CGGCCGCGCA CCGTCGACTC GCTGATCAGC CTCGACTGTG |
| 3241 | CCTTCTAGTT GCCAGCCATC TGTTGTTTGC CCCTCCCCCG TGCCTTCCTT GACCCTGGAA |
| 3301 | GGTGCCACTC CCACTGTCCT TTCCTAATAA AATGAGGAAA TTGCATCGCA TTGTCTGAGT |
| 3361 | AGGTGTCATT CTATTCTGGG GGGTGGGGTG GGGCAGGACA GCAAGGGGGA GGATTGGGAA |
| 3421 | GACAATAGCA GGCATGCTGG GGATGCGGTG GGCTCTATGG CTTCTGAGGC GGAAAGAACC |
| 3481 | AGCTGGGGCT CGACTAGAGC ATGGCTACGT AGATAAGTAG CATGGCGGGT TAATCATTAA |
| 3541 | CTACAAGGAA CCCCTAGTGA TGGAGTTGGC CACTCCCTCT CTGCGCGCTC GCTCGCTCAC |
| 3601 | TGAGGCCGGG CGACCAAAGG TCGCCCGACG CCCGGGCGGC CTCAGTGAGC GAGCGAGCGC |
| 3661 | GCAGAGCTTT TTGCAAAAGC CTAGGCCTCC AAAAAAGCCT CCTCACTACT TCTGGAATAG |
| 3721 | CTCAGAGGCC GAGGCGGCCT CGGCCTCTGC ATAAATAAAA AAAATTAGTC AGCCATGGGG |
| 3781 | CGGAGAATGG GCGGAACTGG GCGGAGTTAG GGGCGGGATG GGCGGAGTTA GGGGCGGGAC |
| 3841 | TATGGTTGCT GACTAATTGA GATGCATGCT TTGCATACTT CTGCCTGCTG GGGAGCCTGG |
| 3901 | GGACTTTCCA CACCTGGTTG CTGACTAATT GAGATGCATG CTTTGCATAC TTCTGCCTGC |
| 3961 | TGGGGAGCCT GGGGACTTTC CACACCCTAA CTGACACACA TTCCACAGCT GCATTAATGA |
| 4021 | ATCGGCCAAC GCGCGGGGAG AGGCGGTTTG CGTATTGGGC GCTCTTCCGC TTCCTCGCTC |
| 4081 | ACTGACTCGC TGCGCTCGGT CGTTCGGCTG CGGCGAGCGG TATCAGCTCA CTCAAAGGCG |
| 4141 | GTAATACGGT TATCCACAGA ATCAGGGGAT AACGCAGGAA AGAACATGTG AGCAAAAGGC |
| 4201 | CAGCAAAAGG CCAGGAACCG TAAAAAGGCC GCGTTGCTGG CGTTTTTCCA TAGGCTCCGC |
| 4261 | CCCCCTGACG AGCATCACAA AAATCGACGC TCAAGTCAGA GGTGGCGAAA CCCGACAGGA |
| 4321 | CTATAAAGAT ACCAGGCGTT TCCCCCTGGA AGCTCCCTCG TGCGCTCTCC TGTTCCGACC |
| 4381 | CTGCCGCTTA CCGGATACCT GTCCGCCTTT CTCCCTTCGG GAAGCGTGGC GCTTTCTCAT |
| 4441 | AGCTCACGCT GTAGGTATCT CAGTTCGGTG TAGGTCGTTC GCTCCAAGCT GGGCTGTGTG |
| 4501 | CACGAACCCC CCGTTCAGCC CGACCGCTGC GCCTTATCCG GTAACTATCG TCTTGAGTCC |
| 4561 | AACCCGGTAA GACACGACTT ATCGCCACTG GCAGCAGCCA CTGGTAACAG GATTAGCAGA |
| 4621 | GCGAGGTATG TAGGCGGTGC TACAGAGTTC TTGAAGTGGT GGCCTAACTA CGGCTACACT |
| 4681 | AGAAGAACAG TATTTGGTAT CTGCGCTCTG CTGAAGCCAG TTACCTTCGG AAAAAGAGTT |
| 4741 | GGTAGCTCTT GATCCGGCAA ACAAACCACC GCTGGTAGCG GTGGTTTTTT TGTTTGCAAG |
| 4801 | CAGCAGATTA CGCGCAGAAA AAAAGGATCT CAAGAAGATC CTTTGATCTT TTCTACGGGG |
| 4861 | TCTGACGCTC AGTGGAACGA AAACTCACGT TAAGGGATTT TGGTCATGAG ATTATCAAAA |
| 4921 | AGGATCTTCA CCTAGATCCT TTTAAATTAA AAATGAAGTT TTAAATCAAT CTAAAGTATA |
| 4981 | TATGAGTAAA CTTGGTCTGA CAGTTACCAA TGCTTAATCA GTGAGGCACC TATCTCAGCG |
| 5041 | ATCTGTCTAT TTCGTTCATC CATAGTTGCC TGACTCCTGC AAACCACGTT GTGTCTCAAA |
| 5101 | ATCTCTGATG TTACATTGCA CAAGATAAAA ATATATCATC ATGAACAATA AAACTGTCTG |
| 5161 | CTTACATAAA CAGTAATACA AGGGGTGTTA TGAGCCATAT TCAACGGGAA ACGTCTTGCT |
| 5221 | CGAGGCCGCG ATTAAATTCC AACATGGATG CTGATTTATA TGGGTATAAA TGGGCTCGCG |
| 5281 | ATAATGTCGG GCAATCAGGT GCGACAATCT ATCGATTGTA TGGGAAGCCC GATGCGCCAG |
| 5341 | AGTTGTTTCT GAAACATGGC AAAGGTAGCG TTGCCAATGA TGTTACAGAT GAGATGGTCA |
| 5401 | GACTAAACTG GCTGACGGAA TTTATGCCTC TTCCGACCAT CAAGCATTTT ATCCGTACTC |
| 5461 | CTGATGATGC ATGGTTACTC ACCACTGCGA TCCCCGGGAA AACAGCATTC CAGGTATTAG |
| 5521 | AAGAATATCC TGATTCAGGT GAAAATATTG TTGATGCGCT GGCAGTGTTC CTGCGCCGGT |
| 5581 | TGCATTCGAT TCCTGTTTGT AATTGTCCTT TTAACAGCGA TCGCGTATTT CGTCTCGCTC |
| 5641 | AGGCGCAATC ACGAATGAAT AACGGTTTGG TTGATGCGAG TGATTTTGAT GACGAGCGTA |
| 5701 | ATGGCTGGCC TGTTGAACAA GTCTGGAAAG AAATGCATAA GCTTTTGCCA TTCTCACCGG |
| 5761 | ATTCAGTCGT CACTCATGGT GATTTCTCAC TTGATAACCT TATTTTTGAC GAGGGGAAAT |
| 5821 | TAATAGGTTG TATTGATGTT GGACGAGTCG GAATCGCAGA CCGATACCAG GATCTTGCCA |
| 5881 | TCCTATGGAA CTGCCTCGGT GAGTTTTCTC CTTCATTACA GAAACGGCTT TTTCAAAAAT |
| 5941 | ATGGTATTGA TAATCCTGAT ATGAATAAAT TGCAGTTTCA TTTGATGCTC GATGAGTTTT |
| 6001 | TCTAAGGGCG GCCTGCCACC ATACCCACGC CGAAACAAGC GCTCATGAGC CCGAAGTGGC |
| 6061 | GAGCCCGATC TTCCCCATCG GTGATGTCGG CGATATAGGC GCCAGCAACC GCACCTGTGG |
| 6121 | CGCCGGTGAT GCCGGCCACG ATGCGTCCGG CGTAGAGGAT CTGGCTAGCG ATGACCCTGC |
| 6181 | TGATTGGTTC GCTGACCATT TCCGGGTGCG GGACGGCGTT ACCAGAAACT CAGAAGGTTC |
| 6241 | GTCCAACCAA ACCGACTCTG ACGGCAGTTT ACGAGAGAGA TGATAGGGTC TGCTTCAGGG |
| 6301 | TGACCGATGT AACCATATAC TTAGGCTGGA TCTTCTCCCG CGAATTTTAA CCCTCACCAA |
| 6361 | CTACGAGATA TGAGGTAAGC CAAAAAAGCA CGTAGTGGCG CTCTCCGACT GTTCCCAAAT |
| 6421 | TGTAACTTAT CGTTCCGTGA AGGCCAGAGT TACTTCCCGG CCCTTTCCAT GCGCGCACCA |
| 6481 | TACCCTCCTA GTTCCCCGGT TATCTTTCCG AAGTGGGAGT GAGCGAACCT CCGTTTACGT |
| 6541 | CTTGTTACCA ATGATGTAGC TATGCACTTT GTACAGGGTG CCAACGGGTT TCACAATTCA |
| 6601 | CAGATAGTGG GGATCCCGGC AAAGGGCCTA TATTTGCGGT CCAACTTAGG CGTAAACCTC |
| 6661 | GATGCTACCT ACTCAGACCC ACCTCGCGCG GGGTAAATAA GGCACTCATC CCAGCTGGTT |
| 6721 | CTTGGCGTTC TACGCAGCGA CATGTTTATT AACAGTTGTC TGGCAGCACA AAACTTTTAC |
| 6781 | CATGGTCGTA GAAGCCCCCC AGAGTTAGTT CATACCTAAT GCCACAAATG TGACAGGACG |
| 6841 | CCGATGGGTA CCGGACTTTA GGTCGAGCAC AGTTCGGTAA CGGAGAGACC CTGCGGCGTA |
| 6901 | CTTCATTATG TATATGGAAC GTGCCCAAGT GACGCCAGGC AAGTCTCAGC TGGTTCCTGT |
| 6961 | GTTAGCTCGA GGGTAGACAT ACGAGCTGAT TGAACATGGG TTGGGGGCCT CGAACCGTCG |
| 7021 | AGGACCCCAT AGTACCTCGG AGACCAAGTA GGGCAGCCTA TAGTTTGAAG CAGAACTATT |
| 7081 | TCGGGGGGCG AGCCCTCATC GTCTCTTCTG CGGATGACTC AACACGCTAG GGACGTGAAG |
| 7141 | TCGATTCCTT CGATGGTTAT AAATCAAAGA CTCAGAGTGC TGTCTGGAGC GTGAATCTAA |
| 7201 | CGGTACGTAT CTCGATTGCT CGGTCGCTTT TCGCACTCCG CGAAAGTTCG TACCGCTCAT |
| 7261 | TCACTAGGTT GCGAAGCCTA TGCTGATATA TGAATCCAAA CTAGAGCAGG GCTCTTAAGA |
| 7321 | TTCGGAGTTG TAAATACTTA ATACTCCAAT CGGCTTTTAC GTGCACCACC GCGGGCGGCT |
| 7381 | GACAAGGGTC TCACATCGAG AAACAAGACA GTTCCGGGCT GGAAGTAGCG CCGGCTAAGG |
| 7441 | AAGACGCCTG GTACGGCAGG ACTATGAAAC CAGTACAAAG GCAACATCCT CACTTGGGTG |
| 7501 | AACGGAAACG CAGTATTATG GTTACTTTTT GGATACGTGA AACATATCCC ATGGTAGTCC |
| 7561 | TTAGACTTGG GAGTCTATCA CCCCTAGGGC CCATATCTGG AAATAGACGC CAGGTTGAAT |
| 7621 | CCGTATTTGG AGGTACGATG GAACAGTCTG GGTGGGACGT GCTTCATTTA TACCCTGCGC |
| 7681 | AGGCTGGACC GAGGACCGCA AGGTGCGGCG GTGCACAAGC AATTGACAAC TAACCACCGT |
| 7741 | GTATTCATTA TGGTACCAGG AACTTTAAGC CGAGTCAATG AAGCTCGCAT TACAGTGTTT |
| 7801 | ACCGCATCTT GCCGTTACTC ACAAACTGTG ATCCACCACA AGTCAAGCCA TTGCCTCTCT |
| 7861 | GACACGCCGT AAGAATTAAT ATGTAAACTT TGCGCGGGTT GACTGCGATC CGTTCAGTCT |
| 7921 | CGTCCGAGGG CACAATCCTA TTCCCATTTG TATGTTCAGC TAACTTCTAC CCATCCCCCG |
| 7981 | AAGTTAAGTA GGTCGTGAGA TGCCATGGAG GCTCTCGTTC ATCCCGTGGG ACATCAAGCT |
| 8041 | TCCCCTTGAT AAAGCACCCC GCTCGGGTGT AGCAGAGAAG ACGCCTTCTG AATTGTGCAA |
| 8101 | TCCCTCCACC TTATCTAAGC TTGCTACCAA TAATTAGCAT TTTTGCCTTG CGACAGACCT |
| 8161 | CCTACTTAGA TTGCCACACA TTGAGCTAGT CAGTGAGCGA TAAGCTTGAC GCGCTTTCAA |
| 8221 | GGGTCGCGAG TACGTGAACT AAGGCTCCGG ACAGGACTAT ATACTTGGGT TTGATCTCGC |
| 8281 | CCCGACAACT GCAAACCTCA ACTTTTTTAG ATTATATGGT TAGCCGAAGT TGCACGAGGT |
| 8341 | GGCGTCCGCG GACTGCTCCC CGAGTGTGGC TCTTTCATCT GACAACGTGC AACCCCTATC |
| 8401 | GCGGCCGATT GTTTCTGCGG ACGATGTTGT CCTCATAGTT TGGGCATGTT TCCCTTGTAG |
| 8461 | GTGTGAAACC ACTTAGCTTC GCGCCGTAGT CCCAATGAAA AACCTATGGA CTTTGTTTTG |
| 8521 | GGTAGCACCA GGAATCTGAA CCGTGTGAAT GTGGACGTCG CGCGCGTAGA CCTTTATCTC |
| 8581 | CGGTTCAAGC TAGGGATGTG GCTGCATGCT ACGTTGTCAC ACCTACACTG CTCGAAGTAA |
| 8641 | ATATGCGAAG CGCGCGGCCT GGCCGGAGGC GTTCCGCGCC GCCACGTGTT CGTTAACTGT |
| 8701 | TGATTGGTGG CACATAAGCA ATATCGTAGT CCGTCAAATT CAGCTCTGTT ATCCCGGGCG |
| 8761 | TTATGTGTCA AATGGCGTAG AACGGGATTG ACTGTTTGAC GGTAGGGTGA CCTAAGCCAG |
| 8821 | ATGCTACACA ATTAGGCTTG TACATATTGT CGTTAGAACG CGGCTACAAT TAATACATAA |
| 8881 | CCTTATGTAT CATACACATA CGATTTAGGT GACACTATAG AATACACGGA ATTAATTC. |

TABLE 1
Features of the VMD2.BEST1.WPRE.pA plasmid sequence

| | | Mini- | Maxi- | | Direc- |
|---|---|---|---|---|---|
| Name | Type | mum | mum | Length | tion |
| 130 bp AAV2 | LTR | 4 | 133 | 130 | forward |
| 5′ITR | | | | | |
| VMD2 promoter | promoter | 189 | 811 | 623 | forward |
| Kozak | Kozak | 812 | 821 | 10 | forward |
| BEST1 | CDS | 818 | 2,575 | 1,758 | forward |
| WPRE | WPRE | 2,606 | 3,198 | 593 | forward |
| bGH pA | polyA_signal | 3,220 | 3,488 | 269 | forward |
| 112 bp AAV2 | LTR | 3,546 | 3,657 | 112 | reverse |
| 3′ITR | | | | | |
| pBR322 rep | rep_origin | 4,230 | 4,849 | 620 | reverse |
| origin | | | | | |
| AphR (KanR) | CDS | 5,190 | 6,005 | 816 | forward |
| Randomly | Stuffer | 6,306 | 8,805 | 2,500 | none |
| generated | | | | | |
| stuffer sequence | | | | | |

In some embodiments, the AAV viral delivery vector comprising a nucleic acid sequence comprising a sequence encoding a VMD2 promoter, a sequence encoding a BEST1 protein, a sequence encoding an intron, a sequence encoding an exon and a sequence encoding a WPRE. An exemplary AAV viral delivery vector of the disclosure comprises a nucleic acid sequence encoding a VMD2.IntEx.BEST1.WPRE.pA sequence comprising or consisting of the nucleic acid sequence of:

| (SEQ ID NO: 14) | |
|---|---|
| 1 | TAGCTGCGCG CTCGCTCGCT CACTGAGGCC GCCCGGGCAA AGCCCGGGCG TCGGGCGACC |
| 61 | TTTGGTCGCC CGGCCTCAGT GAGCGAGCGA GCGCGCAGAG AGGGAGTGGC CAACTCCATC |
| 121 | ACTAGGGGTT CCTTGTAGTT AATGATTAAC CCGCCATGCT ACTTATCTAC GTAGCCATGC |
| 181 | TCTAGGTAAA TTCTGTCATT TTACTAGGGT GATGAAATTC CCAAGCAACA CCATCCTTTT |
| 241 | CAGATAAGGG CACTGAGGCT GAGAGAGGAG CTGAAACCTA CCCGGGGTCA CCACACACAG |
| 301 | GTGGCAAGGC TGGGACCAGA AACCAGGACT GTTGACTGCA GCCCGGTATT CATTCTTTCC |
| 361 | ATAGCCCACA GGGCTGTCAA AGACCCCAGG GCCTAGTCAG AGGCTCCTCC TTCCTGGAGA |
| 421 | GTTCCTGGCA CAGAAGTTGA AGCTCAGCAC AGCCCCCTAA CCCCCAACTC TCTCTGCAAG |
| 481 | GCCTCAGGGG TCAGAACACT GGTGGAGCAG ATCCTTTAGC CTCTGGATTT TAGGGCCATG |
| 541 | GTAGAGGGGG TGTTGCCCTA AATTCCAGCC CTGGTCTCAG CCCAACACCC TCCAAGAAGA |
| 601 | AATTAGAGGG GCCATGGCCA GGCTGTGCTA GCCGTTGCTT CTGAGCAGAT TACAAGAAGG |
| 661 | GACTAAGACA AGGACTCCTT TGTGGAGGTC CTGGCTTAGG GAGTCAAGTG ACGGCGGCTC |
| 721 | AGCACTCACG TGGGCAGTGC CAGCCTCTAA GAGTGGGCAG GGGCACTGGC CACAGAGTCC |
| 781 | CAGGGAGTCC CACCAGCCTA GTCGCCAGAC CGGGTGCCGC AGGGGGACGG CTGCCTTCGG |
| 841 | GGGGGACGGG GCAGGGCGGG GTTCGGCTTC TGGCGTGTGA CCGGCGGCTC TAGAGCCTCT |
| 901 | GCTAACCATG TTCATGCCTT CTTCTTTTTC CTACAGCTCC TGGGCAACGT GCTGGTTATT |
| 961 | GTGCTGTCTC ATCATTTTGG CAAAGAATTG GCACCATGAC CATCACTTAC ACAAGCCAAG |
| 1021 | TGGCTAATGC CCGCTTAGGC TCCTTCTCCC GCCTGCTGCT GTGCTGGCGG GGCAGCATCT |
| 1081 | ACAAGCTGCT ATATGGCGAG TTCTTAATCT TCCTGCTCTG CTACTACATC ATCCGCTTTA |
| 1141 | TTTATAGGCT GGCCCTCACG GAAGAACAAC AGCTGATGTT TGAGAAACTG ACTCTGTATT |
| 1201 | GCGACAGCTA CATCCAGCTC ATCCCCATTT CCTTCGTGCT GGGCTTCTAC GTGACGCTGG |
| 1261 | TCGTGACCCG CTGGTGGAAC CAGTACGAGA ACCTGCCGTG GCCCGACCGC CTCATGAGCC |
| 1321 | TGGTGTCGGG CTTCGTCGAA GGCAAGGACG AGCAAGGCCG GCTGCTGCGG CGCACGCTCA |
| 1381 | TCCGCTACGC CAACCTGGGC AACGTGCTCA TCCTGCGCAG CGTCAGCACC GCAGTCTACA |
| 1441 | AGCGCTTCCC CAGCGCCCAG CACCTGGTGC AAGCAGGCTT TATGACTCCG GCAGAACACA |
| 1501 | AGCAGTTGGA GAAACTGAGC CTACCACACA ACATGTTCTG GGTGCCCTGG GTGTGGTTTG |
| 1561 | CCAACCTGTC AATGAAGGCG TGGCTTGGAG GTCGAATCCG GGACCCTATC CTGCTCCAGA |
| 1621 | GCCTGCTGAA CGAGATGAAC ACCTTGCGTA CTCAGTGTGG ACACCTGTAT GCCTACGACT |
| 1681 | GGATTAGTAT CCCACTGGTG TATACACAGG TGGTGACTGT GGCGGTGTAC AGCTTCTTCC |
| 1741 | TGACTTGTCT AGTTGGGCGG CAGTTTCTGA ACCCAGCCAA GGCCTACCCT GGCCATGAGC |
| 1801 | TGGACCTCGT TGTGCCCGTC TTCACGTTCC TGCAGTTCTT CTTCTATGTT GGCTGGCTGA |
| 1861 | AGGTGGCAGA GCAGCTCATC AACCCCTTTG GAGAGGATGA TGATGATTTT GAGACCAACT |
| 1921 | GGATTGTCGA CAGGAATTTG CAGGTGTCCC TGTTGGCTGT GGATGAGATG CACCAGGACC |
| 1981 | TGCCTCGGAT GGAGCCGGAC ATGTACTGGA ATAAGCCCGA GCCACAGCCC CCCTACACAG |
| 2041 | CTGCTTCCGC CCAGTTCCGT CGAGCCTCCT TTATGGGCTC CACCTTCAAC ATCAGCCTGA |
| 2101 | ACAAAGAGGA GATGGAGTTC CAGCCCAATC AGGAGGACGA GGAGGATGCT CACGCTGGCA |
| 2161 | TCATTGGCCG CTTCCTAGGC CTGCAGTCCC ATGATCACCA TCCTCCCAGG GCAAACTCAA |
| 2221 | GGACCAAACT ACTGTGGCCC AAGAGGGAAT CCCTTCTCCA CGAGGGCCTG CCCAAAAACC |
| 2281 | ACAAGGCAGC CAAACAGAAC GTTAGGGGCC AGGAAGACAA CAAGGCCTGG AAGCTTAAGG |
| 2341 | CTGTGGACGC CTTCAAGTCT GCCCCACTGT ATCAGAGGCC AGGCTACTAC AGTGCCCCAC |
| 2401 | AGACGCCCCT CAGCCCCACT CCCATGTTCT TCCCCCTAGA ACCATCAGCG CCGTCAAAGC |
| 2461 | TTCACAGTGT CACAGGCATA GACACCAAAG ACAAAAGCTT AAAGACTGTG AGTTCTGGGG |
| 2521 | CCAAGAAAAG TTTTGAATTG CTCTCAGAGA GCGATGGGGC CTTGATGGAG CACCCAGAAG |
| 2581 | TATCTCAAGT GAGGAGGAAA ACTGTGGAGT TTAACCTGAC GGATATGCCA GAGATCCCCG |
| 2641 | AAAATCACCT CAAAGAACCT TTGGAACAAT CACCAACCAA CATACACACT ACACTCAAAG |
| 2701 | ATCACATGGA TCCTTATTGG GCCTTGGAAA ACAGGGATGA AGCACATTCC TAATCTAGCG |
| 2761 | GCCGCGAATT CGATATCAAG CTTATCGATA ATCAACCTCT GGATTACAAA ATTTGTGAAA |
| 2821 | GATTGACTGG TATTCTTAAC TATGTTGCTC CTTTTACGCT ATGTGGATAC GCTGCTTTAA |
| 2881 | TGCCTTTGTA TCATGCTATT GCTTCCCGTA TGGCTTTCAT TTTCTCCTCC TTGTATAAAT |
| 2941 | CCTGGTTGCT GTCTCTTTAT GAGGAGTTGT GGCCCGTTGT CAGGCAACGT GGCGTGGTGT |
| 3001 | GCACTGTGTT TGCTGACGCA ACCCCCACTG GTTGGGGCAT TGCCACCACC TGTCAGCTCC |
| 3061 | TTTCCGGGAC TTTCGCTTTC CCCCTCCCTA TTGCCACGGC GGAACTCATC GCCGCCTGCC |
| 3121 | TTGCCCGCTG CTGGACAGGG GCTCGGCTGT TGGGCACTGA CAATTCCGTG GTGTTGTCGG |
| 3181 | GGAAATCATC GTCCTTTCCT TGGCTGCTCG CCTGTGTTGC CACCTGGATT CTGCGCGGGA |
| 3241 | CGTCCTTCTG CTACGTCCCT TCGGCCCTCA ATCCAGCGGA CCTTCCTTCC CGCGGCCTGC |
| 3301 | TGCCGGCTCT GCGGCCTCTT CCGCGTCTTC GCCTTCGCCC TCAGACGAGT CGGATCTCCC |
| 3361 | TTTGGGCCGC CTCCCCGGCG GCCGCGCACC GTCGACTCGC TGATCAGCCT CGACTGTGCC |
| 3421 | TTCTAGTTGC CAGCCATCTG TTGTTTGCCC CTCCCCCGTG CCTTCCTTGA CCCTGGAAGG |
| 3481 | TGCCACTCCC ACTGTCCTTT CCTAATAAAA TGAGGAAATT GCATCGCATT GTCTGAGTAG |
| 3541 | GTGTCATTCT ATTCTGGGGG GTGGGGTGGG GCAGGACAGC AAGGGGGAGG ATTGGGAAGA |
| 3601 | CAATAGCAGG CATGCTGGGG ATGCGGTGGG CTCTATGGCT TCTGAGGCGG AAAGAACCAG |
| 3661 | CTGGGGCTCG ACTAGAGCAT GGCTACGTAG ATAAGTAGCA TGGCGGGTTA ATCATTAACT |
| 3721 | ACAAGGAACC CCTAGTGATG GAGTTGGCCA CTCCCTCTCT GCGCGCTCGC TCGCTCACTG |
| 3781 | AGGCCGGGCG ACCAAAGGTC GCCCGACGCC CGGGCGGCCT CAGTGAGCGA GCGAGCGCGC |
| 3841 | AGAGCTTTTT GCAAAAGCCT AGGCCTCCAA AAAAGCCTCC TCACTACTTC TGGAATAGCT |
| 3901 | CAGAGGCCGA GGCGGCCTCG GCCTCTGCAT AAATAAAAAA AATTAGTCAG CCATGGGGCG |
| 3961 | GAGAATGGGC GGAACTGGGC GGAGTTAGGG GCGGGATGGG CGGAGTTAGG GGCGGGACTA |
| 4021 | TGGTTGCTGA CTAATTGAGA TGCATGCTTT GCATACTTCT GCCTGCTGGG GAGCCTGGGG |
| 4081 | ACTTTCCACA CCTGGTTGCT GACTAATTGA GATGCATGCT TTGCATACTT CTGCCTGCTG |
| 4141 | GGGAGCCTGG GGACTTTCCA CACCCTAACT GACACACATT CCACAGCTGC ATTAATGAAT |
| 4201 | CGGCCAACGC GCGGGGAGAG GCGGTTTGCG TATTGGGCGC TCTTCCGCTT CCTCGCTCAC |
| 4261 | TGACTCGCTG CGCTCGGTCG TTCGGCTGCG GCGAGCGGTA TCAGCTCACT CAAAGGCGGT |
| 4321 | AATACGGTTA TCCACAGAAT CAGGGGATAA CGCAGGAAAG AACATGTGAG CAAAAGGCCA |
| 4381 | GCAAAAGGCC AGGAACCGTA AAAAGGCCGC GTTGCTGGCG TTTTTCCATA GGCTCCGCCC |
| 4441 | CCCTGACGAG CATCACAAAA ATCGACGCTC AAGTCAGAGG TGGCGAAACC CGACAGGACT |
| 4501 | ATAAAGATAC CAGGCGTTTC CCCCTGGAAG CTCCCTCGTG CGCTCTCCTG TTCCGACCCT |
| 4561 | GCCGCTTACC GGATACCTGT CCGCCTTTCT CCCTTCGGGA AGCGTGGCGC TTTCTCATAG |
| 4621 | CTCACGCTGT AGGTATCTCA GTTCGGTGTA GGTCGTTCGC TCCAAGCTGG GCTGTGTGCA |
| 4681 | CGAACCCCCC GTTCAGCCCG ACCGCTGCGC CTTATCCGGT AACTATCGTC TTGAGTCCAA |
| 4741 | CCCGGTAAGA CACGACTTAT CGCCACTGGC AGCAGCCACT GGTAACAGGA TTAGCAGAGC |
| 4801 | GAGGTATGTA GGCGGTGCTA CAGAGTTCTT GAAGTGGTGG CCTAACTACG GCTACACTAG |
| 4861 | AAGAACAGTA TTTGGTATCT GCGCTCTGCT GAAGCCAGTT ACCTTCGGAA AAAGAGTTGG |
| 4921 | TAGCTCTTGA TCCGGCAAAC AAACCACCGC TGGTAGCGGT GGTTTTTTTG TTTGCAAGCA |
| 4981 | GCAGATTACG CGCAGAAAAA AAGGATCTCA AGAAGATCCT TTGATCTTTT CTACGGGGTC |
| 5041 | TGACGCTCAG TGGAACGAAA ACTCACGTTA AGGGATTTTG GTCATGAGAT TATCAAAAAG |
| 5101 | GATCTTCACC TAGATCCTTT TAAATTAAAA ATGAAGTTTT AAATCAATCT AAAGTATATA |
| 5161 | TGAGTAAACT TGGTCTGACA GTTACCAATG CTTAATCAGT GAGGCACCTA TCTCAGCGAT |
| 5221 | CTGTCTATTT CGTTCATCCA TAGTTGCCTG ACTCCTGCAA ACCACGTTGT GTCTCAAAAT |
| 5281 | CTCTGATGTT ACATTGCACA AGATAAAAAT ATATCATCAT GAACAATAAA ACTGTCTGCT |
| 5341 | TACATAAACA GTAATACAAG GGGTGTTATG AGCCATATTC AACGGGAAAC GTCTTGCTCG |
| 5401 | AGGCCGCGAT TAAATTCCAA CATGGATGCT GATTTATATG GGTATAAATG GGCTCGCGAT |
| 5461 | AATGTCGGGC AATCAGGTGC GACAATCTAT CGATTGTATG GGAAGCCCGA TGCGCCAGAG |
| 5521 | TTGTTTCTGA AACATGGCAA AGGTAGCGTT GCCAATGATG TTACAGATGA GATGGTCAGA |
| 5581 | CTAAACTGGC TGACGGAATT TATGCCTCTT CCGACCATCA AGCATTTTAT CCGTACTCCT |
| 5641 | GATGATGCAT GGTTACTCAC CACTGCGATC CCCGGGAAAA CAGCATTCCA GGTATTAGAA |
| 5701 | GAATATCCTG ATTCAGGTGA AAATATTGTT GATGCGCTGG CAGTGTTCCT GCGCCGGTTG |
| 5761 | CATTCGATTC CTGTTTGTAA TTGTCCTTTT AACAGCGATC GCGTATTTCG TCTCGCTCAG |
| 5821 | GCGCAATCAC GAATGAATAA CGGTTTGGTT GATGCGAGTG ATTTTGATGA CGAGCGTAAT |
| 5881 | GGCTGGCCTG TTGAACAAGT CTGGAAAGAA ATGCATAAGC TTTTGCCATT CTCACCGGAT |
| 5941 | TCAGTCGTCA CTCATGGTGA TTTCTCACTT GATAACCTTA TTTTTGACGA GGGGAAATTA |
| 6001 | ATAGGTTGTA TTGATGTTGG ACGAGTCGGA ATCGCAGACC GATACCAGGA TCTTGCCATC |
| 6061 | CTATGGAACT GCCTCGGTGA GTTTTCTCCT TCATTACAGA AACGGCTTTT TCAAAAATAT |
| 6121 | GGTATTGATA ATCCTGATAT GAATAAATTG CAGTTTCATT TGATGCTCGA TGAGTTTTTC |
| 6181 | TAAGGGCGGC CTGCCACCAT ACCCACGCCG AAACAAGCGC TCATGAGCCC GAAGTGGCGA |
| 6241 | GCCCGATCTT CCCCATCGGT GATGTCGGCG ATATAGGCGC CAGCAACCGC ACCTGTGGCG |
| 6301 | CCGGTGATGC CGGCCACGAT GCGTCCGGCG TAGAGGATCT GGCTAGCGAT GACCCTGCTG |
| 6361 | ATTGGTTCGC TGACCATTTC CGGGTGCGGG ACGGCGTTAC CAGAAACTCA GAAGGTTCGT |
| 6421 | CCAACCAAAC CGACTCTGAC GGCAGTTTAC GAGAGAGATG ATAGGGTCTG CTTCAGGGTG |
| 6481 | ACCGATGTAA CCATATACTT AGGCTGGATC TTCTCCCGCG AATTTTAACC CTCACCAACT |
| 6541 | ACGAGATATG AGGTAAGCCA AAAAAGCACG TAGTGGCGCT CTCCGACTGT TCCCAAATTG |
| 6601 | TAACTTATCG TTCCGTGAAG GCCAGAGTTA CTTCCCGGCC CTTTCCATGC GCGCACCATA |
| 6661 | CCCTCCTAGT TCCCCGGTTA TCTTTCCGAA GTGGGAGTGA GCGAACCTCC GTTTACGTCT |
| 6721 | TGTTACCAAT GATGTAGCTA TGCACTTTGT ACAGGGTGCC AACGGGTTTC ACAATTCACA |
| 6781 | GATAGTGGGG ATCCCGGCAA AGGGCCTATA TTTGCGGTCC AACTTAGGCG TAAACCTCGA |
| 6841 | TGCTACCTAC TCAGACCCAC CTCGCGCGGG GTAAATAAGG CACTCATCCC AGCTGGTTCT |
| 6901 | TGGCGTTCTA CGCAGCGACA TGTTTATTAA CAGTTGTCTG GCAGCACAAA ACTTTTACCA |
| 6961 | TGGTCGTAGA AGCCCCCCAG AGTTAGTTCA TACCTAATGC CACAAATGTG ACAGGACGCC |
| 7021 | GATGGGTACC GGACTTTAGG TCGAGCACAG TTCGGTAACG GAGAGACCCT GCGGCGTACT |
| 7081 | TCATTATGTA TATGGAACGT GCCCAAGTGA CGCCAGGCAA GTCTCAGCTG GTTCCTGTGT |
| 7141 | TAGCTCGAGG GTAGACATAC GAGCTGATTG AACATGGGTT GGGGGCCTCG AACCGTCGAG |
| 7201 | GACCCCATAG TACCTCGGAG ACCAAGTAGG GCAGCCTATA GTTTGAAGCA GAACTATTTC |
| 7261 | GGGGGGCGAG CCCTCATCGT CTCTTCTGCG GATGACTCAA CACGCTAGGG ACGTGAAGTC |
| 7321 | GATTCCTTCG ATGGTTATAA ATCAAAGACT CAGAGTGCTG TCTGGAGCGT GAATCTAACG |
| 7381 | GTACGTATCT CGATTGCTCG GTCGCTTTTC GCACTCCGCG AAAGTTCGTA CCGCTCATTC |
| 7441 | ACTAGGTTGC GAAGCCTATG CTGATATATG AATCCAAACT AGAGCAGGGC TCTTAAGATT |
| 7501 | CGGAGTTGTA AATACTTAAT ACTCCAATCG GCTTTTACGT GCACCACCGC GGGCGGCTGA |
| 7561 | CAAGGGTCTC ACATCGAGAA ACAAGACAGT TCCGGGCTGG AAGTAGCGCC GGCTAAGGAA |
| 7621 | GACGCCTGGT ACGGCAGGAC TATGAAACCA GTACAAAGGC AACATCCTCA CTTGGGTGAA |
| 7681 | CGGAAACGCA GTATTATGGT TACTTTTTGG ATACGTGAAA CATATCCCAT GGTAGTCCTT |
| 7741 | AGACTTGGGA GTCTATCACC CCTAGGGCCC ATATCTGGAA ATAGACGCCA GGTTGAATCC |
| 7801 | GTATTTGGAG GTACGATGGA ACAGTCTGGG TGGGACGTGC TTCATTTATA CCCTGCGCAG |
| 7861 | GCTGGACCGA GGACCGCAAG GTGCGGCGGT GCACAAGCAA TTGACAACTA ACCACCGTGT |
| 7921 | ATTCATTATG GTACCAGGAA CTTTAAGCCG AGTCAATGAA GCTCGCATTA CAGTGTTTAC |
| 7981 | CGCATCTTGC CGTTACTCAC AAACTGTGAT CCACCACAAG TCAAGCCATT GCCTCTCTGA |
| 8041 | CACGCCGTAA GAATTAATAT GTAAACTTTG CGCGGGTTGA CTGCGATCCG TTCAGTCTCG |
| 8101 | TCCGAGGGCA CAATCCTATT CCCATTTGTA TGTTCAGCTA ACTTCTACCC ATCCCCCGAA |
| 8161 | GTTAAGTAGG TCGTGAGATG CCATGGAGGC TCTCGTTCAT CCCGTGGGAC ATCAAGCTTC |
| 8221 | CCCTTGATAA AGCACCCCGC TCGGGTGTAG CAGAGAAGAC GCCTTCTGAA TTGTGCAATC |
| 8281 | CCTCCACCTT ATCTAAGCTT GCTACCAATA ATTAGCATTT TTGCCTTGCG ACAGACCTCC |
| 8341 | TACTTAGATT GCCACACATT GAGCTAGTCA GTGAGCGATA AGCTTGACGC GCTTTCAAGG |
| 8401 | GTCGCGAGTA CGTGAACTAA GGCTCCGGAC AGGACTATAT ACTTGGGTTT GATCTCGCCC |
| 8461 | CGACAACTGC AAACCTCAAC TTTTTTAGAT TATATGGTTA GCCGAAGTTG CACGAGGTGG |
| 8521 | CGTCCGCGGA CTGCTCCCCG AGTGTGGCTC TTTCATCTGA CAACGTGCAA CCCCTATCGC |
| 8581 | GGCCGATTGT TTCTGCGGAC GATGTTGTCC TCATAGTTTG GGCATGTTTC CCTTGTAGGT |
| 8641 | GTGAAACCAC TTAGCTTCGC GCCGTAGTCC CAATGAAAAA CCTATGGACT TTGTTTTGGG |
| 8701 | TAGCACCAGG AATCTGAACC GTGTGAATGT GGACGTCGCG CGCGTAGACC TTTATCTCCG |
| 8761 | GTTCAAGCTA GGGATGTGGC TGCATGCTAC GTTGTCACAC CTACACTGCT CGAAGTAAAT |
| 8821 | ATGCGAAGCG CGCGGCCTGG CCGGAGGCGT TCCGCGCCGC CACGTGTTCG TTAACTGTTG |
| 8881 | ATTGGTGGCA CATAAGCAAT ATCGTAGTCC GTCAAATTCA GCTCTGTTAT CCCGGGCGTT |
| 8941 | ATGTGTCAAA TGGCGTAGAA CGGGATTGAC TGTTTGACGG TAGGGTGACC TAAGCCAGAT |
| 9001 | GCTACACAAT TAGGCTTGTA CATATTGTCG TTAGAACGCG GCTACAATTA ATACATAACC |
| 9061 | TTATGTATCA TACACATACG ATTTAGGTGA CACTATAGAA TACACGGAAT TAATTC. |

TABLE 2
Features of the VMD2.IntEx.BEST1.WPRE.pA plasmid sequence

| | | Mini- | Maxi- | | Direc- |
|---|---|---|---|---|---|
| Name | Type | mum | mum | Length | tion |
| AAV2 ITR | LTR | 4 | 133 | 130 | forward |
| −585 to +38 | promoter | 189 | 811 | 623 | forward |
| VMD2 promoter | | | | | |
| Intron | intron | 814 | 936 | 123 | forward |
| Exon | exon | 937 | 989 | 53 | forward |
| Kozak | Kozak | 990 | 999 | 10 | forward |
| BEST1 | CDS | 996 | 2753 | 1758 | forward |
| NotI | RBS | 2758 | 2765 | 8 | none |
| WPRE | WPRE | 2784 | 3376 | 593 | forward |
| NotI | RBS | 3378 | 3385 | 8 | none |
| bGH pA | polyA_signal | 3398 | 3666 | 269 | forward |
| AAV2 ITR | LTR | 3724 | 3844 | 121 | reverse |
| pBR322 rep origin | rep_origin | 4408 | 5027 | 620 | reverse |
| AphR (KanR) | CDS | 5368 | 6183 | 816 | forward |
| BstEII | RBS | 6477 | 6483 | 7 | none |
| Randomly generated | Stuffer | 6484 | 8983 | 2500 | none |
| stuffer sequence | | | | | |
| BstEII | RBS | 8984 | 8990 | 7 | none |

In some embodiments, the AAV viral delivery vector comprises a nucleic acid sequence comprising a sequence encoding a CAG promoter, a sequence encoding a BEST1 protein and a sequence encoding a WPRE. An exemplary AAV viral delivery vector of the disclosure comprising a nucleic acid sequence encoding a CAG.BEST1.WPRE.pA sequence comprises or consists of the nucleic acid sequence of:

| (SEQ Id NO: 15) | |
|---|---|
| 1 | TAGCTGCGCG CTCGCTCGCT CACTGAGGCC GCCCGGGCAA AGCCCGGGCG TCGGGCGACC |
| 61 | TTTGGTCGCC CGGCCTCAGT GAGCGAGCGA GCGCGCAGAG AGGGAGTGGC CAACTCCATC |
| 121 | ACTAGGGGTT CCTTGTAGTT AATGATTAAC CCGCCATGCT ACTTATCTAC GTAGCCATGC |
| 181 | TCTAGGTACC ATTGACGTCA ATAATGACGT ATGTTCCCAT AGTAACGCCA ATAGGGACTT |
| 241 | TCCATTGACG TCAATGGGTG GAGTATTTAC GGTAAACTGC CCACTTGGCA GTACATCAAG |
| 301 | TGTATCATAT GCCAAGTACG CCCCCTATTG ACGTCAATGA CGGTAAATGG CCCGCCTGGC |
| 361 | ATTATGCCCA GTACATGACC TTATGGGACT TTCCTACTTG GCAGTACATC TACGTATTAG |
| 421 | TCATCGCTAT TACCATGGTC GAGGTGAGCC CCACGTTCTG CTTCACTCTC CCCATCTCCC |
| 481 | CCCCCTCCCC ACCCCCAATT TTGTATTTAT TTATTTTTTA ATTATTTTGT GCAGCGATGG |
| 541 | GGGCGGGGGG GGGGGGGGGG CGCGCGCCAG GCGGGGCGGG GCGGGGCGAG GGGCGGGGCG |
| 601 | GGGCGAGGCG GAGAGGTGCG GCGGCAGCCA ATCAGAGCGG CGCGCTCCGA AAGTTTCCTT |
| 661 | TTATGGCGAG GCGGCGGCGG CGGCGGCCCT ATAAAAAGCG AAGCGCGCGG CGGGCGGGAG |
| 721 | TCGCTGCGCG CTGCCTTCGC CCCGTGCCCC GCTCCGCCGC CGCCTCGCGC CGCCCGCCCC |
| 781 | GGCTCTGACT GACCGCGTTA CTCCCACAGG TGAGCGGGCG GGACGGCCCT TCTCCTCCGG |
| 841 | GCTGTAATTA GCGCTTGGTT TAATGACGGC TTGTTTCTTT TCTGTGGCTG CGTGAAAGCC |
| 901 | TTGAGGGGCT CCGGGAGGGC CCTTTGTGCG GGGGGAGCGG CTCGGGGCTG TCCGCGGGGG |
| 961 | GACGGCTGCC TTCGGGGGGG ACGGGGCAGG GCGGGGTTCG GCTTCTGGCG TGTGACCGGC |
| 1021 | GGCTCTAGAG CCTCTGCTAA CCATGTTCAT GCCTTCTTCT TTTTCCTACA GCTCCTGGGC |
| 1081 | AACGTGCTGG TTATTGTGCT GTCTCATCAT TTTGGCAAAG AATTGGATCC GCGGCCGCAG |
| 1141 | CTTGGTACCG CCACCATGAC CATCACTTAC ACAAGCCAAG TGGCTAATGC CCGCTTAGGC |
| 1201 | TCCTTCTCCC GCCTGCTGCT GTGCTGGCGG GGCAGCATCT ACAAGCTGCT ATATGGCGAG |
| 1261 | TTCTTAATCT TCCTGCTCTG CTACTACATC ATCCGCTTTA TTTATAGGCT GGCCCTCACG |
| 1321 | GAAGAACAAC AGCTGATGTT TGAGAAACTG ACTCTGTATT GCGACAGTTA CATCCAGCTC |
| 1381 | ATCCCCATTT CCTTCGTGCT GGGCTTCTAC GTGACGCTGG TCGTGACCCG CTGGTGGAAC |
| 1441 | CAGTACGAGA ACCTGCCGTG GCCCGACCGC CTCATGAGCC TGGTGTCGGG CTTCGTCGAA |
| 1501 | GGCAAGGACG AGCAAGGCCG GCTGCTGCGG CGCACGCTCA TCCGCTACGC CAACCTGGGC |
| 1561 | AACGTGCTCA TCCTGCGCAG CGTCAGCACC GCAGTCTACA AGCGCTTCCC CAGCGCCCAG |
| 1621 | CACCTGGTGC AAGCAGGCTT TATGACTCCG GCAGAACACA AGCAGTTGGA GAAACTGAGC |
| 1681 | CTACCACACA ACATGTTCTG GGTGCCCTGG GTGTGGTTTG CCAACCTGTC AATGAAGGCG |
| 1741 | TGGCTTGGAG GTCGAATCCG GGACCCTATC CTGCTCCAGA GCCTGCTGAA CGAGATGAAC |
| 1801 | ACCTTGCGTA CTCAGTGTGG ACACCTGTAT GCCTACGACT GGATTAGTAT CCCACTGGTG |
| 1861 | TATACACAGG TGGTGACTGT GGCGGTGTAC AGCTTCTTCC TGACTTGTCT AGTTGGGCGG |
| 1921 | CAGTTTCTGA ACCCAGCCAA GGCCTACCCT GGCCATGAGC TGGACCTCGT TGTGCCCGTC |
| 1981 | TTCACGTTCC TGCAGTTCTT CTTCTATGTT GGCTGGCTGA AGGTGGCAGA GCAGCTCATC |
| 2041 | AACCCCTTTG GAGAGGATGA TGATGATTTT GAGACCAACT GGATTGTCGA CAGGAATTTG |
| 2101 | CAGGTGTCCC TGTTGGCTGT GGATGAGATG CACCAGGACC TGCCTCGGAT GGAGCCGGAC |
| 2161 | ATGTACTGGA ATAAGCCCGA GCCACAGCCC CCCTACACAG CTGCTTCCGC CCAGTTCCGT |
| 2221 | CGAGCCTCCT TTATGGGCTC CACCTTCAAC ATCAGCCTGA ACAAAGAGGA GATGGAGTTC |
| 2281 | CAGCCCAATC AGGAGGACGA GGAGGATGCT CACGCTGGCA TCATTGGCCG CTTCCTAGGC |
| 2341 | CTGCAGTCCC ATGATCACCA TCCTCCCAGG GCAAACTCAA GGACCAAACT ACTGTGGCCC |
| 2401 | AAGAGGGAAT CCCTTCTCCA CGAGGGCCTG CCCAAAAACC ACAAGGCAGC CAAACAGAAC |
| 2461 | GTTAGGGGCC AGGAAGACAA CAAGGCCTGG AAGCTTAAGG CTGTGGACGC CTTCAAGTCT |
| 2521 | GCCCCACTGT ATCAGAGGCC AGGCTACTAC AGTGCCCCAC AGACGCCCCT CAGCCCCACT |
| 2581 | CCCATGTTCT TCCCCCTAGA ACCATCAGCG CCGTCAAAGC TTCACAGTGT CACAGGCATA |
| 2641 | GACACCAAAG ACAAAAGCTT AAAGACTGTG AGTTCTGGGG CCAAGAAAAG TTTTGAATTG |
| 2701 | CTCTCAGAGA GCGATGGGGC CTTGATGGAG CACCCAGAAG TATCTCAAGT GAGGAGGAAA |
| 2761 | ACTGTGGAGT TTAACCTGAC GGATATGCCA GAGATCCCCG AAAATCACCT CAAAGAACCT |
| 2821 | TTGGAACAAT CACCAACCAA CATACACACT ACACTCAAAG ATCACATGGA TCCTTATTGG |
| 2881 | GCCTTGGAAA ACAGGGATGA AGCACATTCC TAAGAGCTCA AGCTTATCGA TAATCAACCT |
| 2941 | CTGGATTACA AAATTTGTGA AAGATTGACT GGTATTCTTA ACTATGTTGC TCCTTTTACG |
| 3001 | CTATGTGGAT ACGCTGCTTT AATGCCTTTG TATCATGCTA TTGCTTCCCG TATGGCTTTC |
| 3061 | ATTTTCTCCT CCTTGTATAA ATCCTGGTTG CTGTCTCTTT ATGAGGAGTT GTGGCCCGTT |
| 3121 | GTCAGGCAAC GTGGCGTGGT GTGCACTGTG TTTGCTGACG CAACCCCCAC TGGTTGGGGC |
| 3181 | ATTGCCACCA CCTGTCAGCT CCTTTCCGGG ACTTTCGCTT TCCCCCTCCC TATTGCCACG |
| 3241 | GCGGAACTCA TCGCCGCCTG CCTTGCCCGC TGCTGGACAG GGGCTCGGCT GTTGGGCACT |
| 3301 | GACAATTCCG TGGTGTTGTC GGGGAAATCA TCGTCCTTTC CTTGGCTGCT CGCCTGTGTT |
| 3361 | GCCACCTGGA TTCTGCGCGG GACGTCCTTC TGCTACGTCC CTTCGGCCCT CAATCCAGCG |
| 3421 | GACCTTCCTT CCCGCGGCCT GCTGCCGGCT CTGCGGCCTC TTCCGCGTCT TCGCCTTCGC |
| 3481 | CCTCAGACGA GTCGGATCTC CCTTTGGGCC GCCTCCCCGC ATCGATACCG TCGACTCGCT |
| 3541 | GATCAGCCTC GACTGTGCCT TCTAGTTGCC AGCCATCTGT TGTTTGCCCC TCCCCCGTGC |
| 3601 | CTTCCTTGAC CCTGGAAGGT GCCACTCCCA CTGTCCTTTC CTAATAAAAT GAGGAAATTG |
| 3661 | CATCGCATTG TCTGAGTAGG TGTCATTCTA TTCTGGGGGG TGGGGTGGGG CAGGACAGCA |
| 3721 | AGGGGGAGGA TTGGGAAGAC AATAGCAGGC ATGCTGGGGA TGCGGTGGGC TCTATGGCTT |
| 3781 | CTGAGGCGGA AAGAACCAGC TGGGGCTCGA CTAGAGCATG GCTACGTAGA TAAGTAGCAT |
| 3841 | GGCGGGTTAA TCATTAACTA CAAGGAACCC CTAGTGATGG AGTTGGCCAC TCCCTCTCTG |
| 3901 | CGCGCTCGCT CGCTCACTGA GGCCGGGCGA CCAAAGGTCG CCCGACGCCC GGGCGGCCTC |
| 3961 | AGTGAGCGAG CGAGCGCGCA GAGCTTTTTG CAAAAGCCTA GGCCTCCAAA AAAGCCTCCT |
| 4021 | CACTACTTCT GGAATAGCTC AGAGGCCGAG GCGGCCTCGG CCTCTGCATA AATAAAAAAA |
| 4081 | ATTAGTCAGC CATGGGGCGG AGAATGGGCG GAACTGGGCG GAGTTAGGGG CGGGATGGGC |
| 4141 | GGAGTTAGGG GCGGGACTAT GGTTGCTGAC TAATTGAGAT GCATGCTTTG CATACTTCTG |
| 4201 | CCTGCTGGGG AGCCTGGGGA CTTTCCACAC CTGGTTGCTG ACTAATTGAG ATGCATGCTT |
| 4261 | TGCATACTTC TGCCTGCTGG GGAGCCTGGG GACTTTCCAC ACCCTAACTG ACACACATTC |
| 4321 | CACAGCTGCA TTAATGAATC GGCCAACGCG CGGGGAGAGG CGGTTTGCGT ATTGGGCGCT |
| 4381 | CTTCCGCTTC CTCGCTCACT GACTCGCTGC GCTCGGTCGT TCGGCTGCGG CGAGCGGTAT |
| 4441 | CAGCTCACTC AAAGGCGGTA ATACGGTTAT CCACAGAATC AGGGGATAAC GCAGGAAAGA |
| 4501 | ACATGTGAGC AAAAGGCCAG CAAAAGGCCA GGAACCGTAA AAAGGCCGCG TTGCTGGCGT |
| 4561 | TTTTCCATAG GCTCCGCCCC CCTGACGAGC ATCACAAAAA TCGACGCTCA AGTCAGAGGT |
| 4621 | GGCGAAACCC GACAGGACTA TAAAGATACC AGGCGTTTCC CCCTGGAAGC TCCCTCGTGC |
| 4681 | GCTCTCCTGT TCCGACCCTG CCGCTTACCG GATACCTGTC CGCCTTTCTC CCTTCGGGAA |
| 4741 | GCGTGGCGCT TTCTCATAGC TCACGCTGTA GGTATCTCAG TTCGGTGTAG GTCGTTCGCT |
| 4801 | CCAAGCTGGG CTGTGTGCAC GAACCCCCCG TTCAGCCCGA CCGCTGCGCC TTATCCGGTA |
| 4861 | ACTATCGTCT TGAGTCCAAC CCGGTAAGAC ACGACTTATC GCCACTGGCA GCAGCCACTG |
| 4921 | GTAACAGGAT TAGCAGAGCG AGGTATGTAG GCGGTGCTAC AGAGTTCTTG AAGTGGTGGC |
| 4981 | CTAACTACGG CTACACTAGA AGAACAGTAT TTGGTATCTG CGCTCTGCTG AAGCCAGTTA |
| 5041 | CCTTCGGAAA AAGAGTTGGT AGCTCTTGAT CCGGCAAACA AACCACCGCT GGTAGCGGTG |
| 5101 | GTTTTTTTGT TTGCAAGCAG CAGATTACGC GCAGAAAAAA AGGATCTCAA GAAGATCCTT |
| 5161 | TGATCTTTTC TACGGGGTCT GACGCTCAGT GGAACGAAAA CTCACGTTAA GGGATTTTGG |
| 5221 | TCATGAGATT ATCAAAAAGG ATCTTCACCT AGATCCTTTT AAATTAAAAA TGAAGTTTTA |
| 5281 | AATCAATCTA AAGTATATAT GAGTAAACTT GGTCTGACAG TTACCAATGC TTAATCAGTG |
| 5341 | AGGCACCTAT CTCAGCGATC TGTCTATTTC GTTCATCCAT AGTTGCCTGA CTCCCCGTCG |
| 5401 | TGTAGATAAC TACGATACGG GAGGGCTTAC CATCTGGCCC CAGTGCTGCA ATGATACCGC |
| 5461 | GAGACCCACG CTCACCGGCT CCAGATTTAT CAGCAATAAA CCAGCCAGCC GGAAGGGCCG |
| 5521 | AGCGCAGAAG TGGTCCTGCA ACTTTATCCG CCTCCATCCA GTCTATTAAT TGTTGCCGGG |
| 5581 | AAGCTAGAGT AAGTAGTTCG CCAGTTAATA GTTTGCGCAA CGTTGTTGCC ATTGCTACAG |
| 5641 | GCATCGTGGT GTCACGCTCG TCGTTTGGTA TGGCTTCATT CAGCTCCGGT TCCCAACGAT |
| 5701 | CAAGGCGAGT TACATGATCC CCCATGTTGT GCAAAAAAGC GGTTAGCTCC TTCGGTCCTC |
| 5761 | CGATCGTTGT CAGAAGTAAG TTGGCCGCAG TGTTATCACT CATGGTTATG GCAGCACTGC |
| 5821 | ATAATTCTCT TACTGTCATG CCATCCGTAA GATGCTTTTC TGTGACTGGT GAGTACTCAA |
| 5881 | CCAAGTCATT CTGAGAATAG TGTATGCGGC GACCGAGTTG CTCTTGCCCG GCGTCAATAC |
| 5941 | GGGATAATAC CGCGCCACAT AGCAGAACTT TAAAAGTGCT CATCATTGGA AAACGTTCTT |
| 6001 | CGGGGCGAAA ACTCTCAAGG ATCTTACCGC TGTTGAGATC CAGTTCGATG TAACCCACTC |
| 6061 | GTGCACCCAA CTGATCTTCA GCATCTTTTA CTTTCACCAG CGTTTCTGGG TGAGCAAAAA |
| 6121 | CAGGAAGGCA AAATGCCGCA AAAAAGGGAA TAAGGGCGAC ACGGAAATGT TGAATACTCA |
| 6181 | TACTCTTCCT TTTTCAATAT TATTGAAGCA TTTATCAGGG TTATTGTCTC ATGAGCGGAT |
| 6241 | ACATATTTGA ATGTATTTAG AAAAATAAAC AAATAGGGGT TCCGCGCACA TTTCCCCGAA |
| 6301 | AAGTGCCACC TGACGTCTAA GAAACCATTA TTATCATGAC ATTAACCTAT AAAAATAGGC |
| 6361 | GTATCACGAG GCCCTTTCGT CTCGCGCGTT TCGGTGATGA CGGTGAAAAC CTCTGACACA |
| 6421 | TGCAGCTCCC GGAGACGGTC ACAGCTTGTC TGTAAGCGGA TGCCGGGAGC AGACAAGCCC |
| 6481 | GTCAGGGCGC GTCAGCGGGT GTTGGCGGGT GTCGGGGCTG GCTTAACTAT GCGGCATCAG |
| 6541 | AGCAGATTGT ACTGAGAGTG CACCATTCGA CGCTCTCCCT TATGCGACTC CTGCATTAGG |
| 6601 | AAGCAGCCCA GTAGTAGGTT GAGGCCGTTG AGCACCGCCG CCGCAAGGAA TGGTGCATGC |
| 6661 | AAGGAGATGG CGCCCAACAG TCCCCCGGCC ACGGGGCCTG CCACCATACC CACGCCGAAA |
| 6721 | CAAGCGCTCA TGAGCCCGAA GTGGCGAGCC CGATCTTCCC CATCGGTGAT GTCGGCGATA |
| 6781 | TAGGCGCCAG CAACCGCACC TGTGGCGCCG GTGATGCCGG CCACGATGCG TCCGGCGTAG |
| 6841 | AGGATCTGGC TAGCGATGAC CCTGCTGATT GGTTCGCTGA CCATTTCCGG GTGCGGGACG |
| 6901 | GCGTTACCAG AAACTCAGAA GGTTCGTCCA ACCAAACCGA CTCTGACGGC AGTTTACGAG |
| 6961 | AGAGATGATA GGGTCTGCTT CAGTAAGCCA GATGCTACAC AATTAGGCTT GTACATATTG |
| 7021 | TCGTTAGAAC GCGGCTACAA TTAATACATA ACCTTATGTA TCATACACAT ACGATTTAGG |
| 7081 | TGACACTATA GAATACACGG AATTAATTC. |

TABLE 3
Features of the CAG.BEST1.WPRE.PA plasmid sequence

| | | Mini- | Maxi- | | Direc- |
|---|---|---|---|---|---|
| Name | Type | mum | mum | Length | tion |
| AAV2 ITR | repeat_region | 7,066 | 133 | 177 | forward |
| amp prom | promoter | 6,223 | 6,251 | 29 | reverse |
| AmpR gene | gene | 5,321 | 6,181 | 861 | reverse |
| Bla gene | gene | 5,321 | 5,983 | 663 | reverse |
| ColE1 origin | rep_origin | 4,503 | 5,176 | 674 | forward |
| rep origin | | | | | |
| SV40 origin | origin of | 4,059 | 4,136 | 78 | reverse |
| | replication | | | | |
| AAV2 ITR | repeat_region | 3,864 | 4,000 | 137 | reverse |
| bGH_PA term | terminator | 3,550 | 3,777 | 228 | forward |
| WPRE | misc_feature | 2,932 | 3,520 | 589 | forward |
| Exon 10 | exon | 2,895 | 2,913 | 19 | forward |
| Exon 9 | exon | 2,256 | 2,894 | 639 | forward |
| Exon 8 | exon | 2,104 | 2,255 | 152 | forward |
| Exon 7 | exon | 2,023 | 2,103 | 81 | forward |
| Exon 6 | exon | 1,870 | 2,022 | 153 | forward |
| Exon 5 | exon | 1,792 | 1,869 | 78 | forward |
| Exon 4 | exon | 1,637 | 1,791 | 155 | forward |
| Exon 3 | exon | 1,403 | 1,636 | 234 | forward |
| C > T | modified_base | 1,368 | 1,368 | 1 | forward |
| Exon 2 | exon | 1,308 | 1,402 | 95 | forward |
| hBEST1 CDS | CDS | 1,156 | 2,913 | 1,758 | forward |
| Exon 1 | exon | 1,156 | 1,307 | 152 | forward |
| Kozak | unsure | 1,147 | 1,155 | 9 | forward |
| | Editing History | <1133 | 1,138 | >6 | none |
| | Insertion | | | | |
| CAG promoter | promoter | 189 | 1,129 | 941 | forward |
| 5′ITR on REP1 | LTR | 64 | 183 | 120 | forward |
| official | | | | | |
| sequence file | | | | | |

In some embodiments of the compositions of the disclosure, a vector may comprise a sequence encoding a marker, which may be expressed in a cell when the cell is either in vitro or in vivo. For example, in a vector or nucleic acid sequence of the disclosure, a sequence encoding a marker may be used in place of or may replace a sequence encoding a BEST1 protein of the disclosure (e.g. a sequence comprising a coding sequence of a BEST1 gene). Exemplary markers of the disclosure include, but are not limited to, fluorophore proteins such as GFP, YFP or dsRED as well as various epitope tags such as FLAG, HA, His or Myc. The fluorophore or epitope tag may be fused to the BEST1 coding sequence, for example as an N or C terminal fusion, or may be used in place of BEST1 to characterize a vector of the disclosure. Exemplary uses for a vector containing a marker of the disclosure include, but are not limited to characterizing gene expression, for example levels of expression, or characterizing the cell type specificity of a vector of the disclosure.

An exemplary a vector of the disclosure comprising a marker includes VMD2.GFP.WPRE.pA. A nucleic acid sequence encoding a VMD2.GFP.WPRE.pA construct comprises or consists of:

| (SEQ ID NO: 16) | |
|---|---|
| 1 | TAGCTGCGCG CTCGCTCGCT CACTGAGGCC GCCCGGGCAA AGCCCGGGCG TCGGGCGACC |
| 61 | TTTGGTCGCC CGGCCTCAGT GAGCGAGCGA GCGCGCAGAG AGGGAGTGGC CAACTCCATC |
| 121 | ACTAGGGGTT CCTTGTAGTT AATGATTAAC CCGCCATGCT ACTTATCTAC GTAGCCATGC |
| 181 | TCTAGGTAAA TTCTGTCATT TTACTAGGGT GATGAAATTC CCAAGCAACA CCATCCTTTT |
| 241 | CAGATAAGGG CACTGAGGCT GAGAGAGGAG CTGAAACCTA CCCGGGGTCA CCACACACAG |
| 301 | GTGGCAAGGC TGGGACCAGA AACCAGGACT GTTGACTGCA GCCCGGTATT CATTCTTTCC |
| 361 | ATAGCCCACA GGGCTGTCAA AGACCCCAGG GCCTAGTCAG AGGCTCCTCC TTCCTGGAGA |
| 421 | GTTCCTGGCA CAGAAGTTGA AGCTCAGCAC AGCCCCCTAA CCCCCAACTC TCTCTGCAAG |
| 481 | GCCTCAGGGG TCAGAACACT GGTGGAGCAG ATCCTTTAGC CTCTGGATTT TAGGGCCATG |
| 541 | GTAGAGGGGG TGTTGCCCTA AATTCCAGCC CTGGTCTCAG CCCAACACCC TCCAAGAAGA |
| 601 | AATTAGAGGG GCCATGGCCA GGCTGTGCTA GCCGTTGCTT CTGAGCAGAT TACAAGAAGG |
| 661 | GACTAAGACA AGGACTCCTT TGTGGAGGTC CTGGCTTAGG GAGTCAAGTG ACGGCGGCTC |
| 721 | AGCACTCACG TGGGCAGTGC CAGCCTCTAA GAGTGGGCAG GGGCACTGGC CACAGAGTCC |
| 781 | CAGGGAGTCC CACCAGCCTA GTCGCCAGAC CGGCACCATG AGCAAGGGCG AGGAACTGTT |
| 841 | CACTGGCGTG GTCCCAATTC TCGTGGAACT GGATGGCGAT GTGAATGGGC ACAAATTTTC |
| 901 | TGTCAGCGGA GAGGGTGAAG GTGATGCCAC ATACGGAAAG CTCACCCTGA AATTCATCTG |
| 961 | CACCACTGGA AAGCTCCCTG TGCCATGGCC AACACTGGTC ACTACCCTGA CCTATGGCGT |
| 1021 | GCAGTGCTTT TCCAGATACC CAGACCATAT GAAGCAGCAT GACTTTTTCA AGAGCGCCAT |
| 1081 | GCCCGAGGGC TATGTGCAGG AGAGAACCAT CTTTTTCAAA GATGACGGGA ACTACAAGAC |
| 1141 | CCGCGCTGAA GTCAAGTTCG AAGGTGACAC CCTGGTGAAT AGAATCGAGC TGAAGGGCAT |
| 1201 | TGACTTTAAG GAGGATGGAA ACATTCTCGG CCACAAGCTG GAATACAACT ATAACTCCCA |
| 1261 | CAATGTGTAC ATCATGGCCG ACAAGCAAAA GAATGGCATC AAGGTCAACT TCAAGATCAG |
| 1321 | ACACAACATT GAGGATGGAT CCGTGCAGCT GGCCGACCAT TATCAACAGA ACACTCCAAT |
| 1381 | CGGCGACGGC CCTGTGCTCC TCCCAGACAA CCATTACCTG TCCACCCAGT CTGCCCTGTC |
| 1441 | TAAAGATCCC AACGAAAAGA GAGACCACAT GGTCCTGCTG GAGTTTGTGA CCGCTGCTGG |
| 1501 | GATCACACAT GGCATGGACG AGCTGTACAA GTGAAAGCTT ATCGATAATC AACCTCTGGA |
| 1561 | TTACAAAATT TGTGAAAGAT TGACTGGTAT TCTTAACTAT GTTGCTCCTT TTACGCTATG |
| 1621 | TGGATACGCT GCTTTAATGC CTTTGTATCA TGCTATTGCT TCCCGTATGG CTTTCATTTT |
| 1681 | CTCCTCCTTG TATAAATCCT GGTTGCTGTC TCTTTATGAG GAGTTGTGGC CCGTTGTCAG |
| 1741 | GCAACGTGGC GTGGTGTGCA CTGTGTTTGC TGACGCAACC CCCACTGGTT GGGGCATTGC |
| 1801 | CACCACCTGT CAGCTCCTTT CCGGGACTTT CGCTTTCCCC CTCCCTATTG CCACGGCGGA |
| 1861 | ACTCATCGCC GCCTGCCTTG CCCGCTGCTG GACAGGGGCT CGGCTGTTGG GCACTGACAA |
| 1921 | TTCCGTGGTG TTGTCGGGGA AATCATCGTC CTTTCCTTGG CTGCTCGCCT GTGTTGCCAC |
| 1981 | CTGGATTCTG CGCGGGACGT CCTTCTGCTA CGTCCCTTCG GCCCTCAATC CAGCGGACCT |
| 2041 | TCCTTCCCGC GGCCTGCTGC CGGCTCTGCG GCCTCTTCCG CGTCTTCGCC TTCGCCCTCA |
| 2101 | GACGAGTCGG ATCTCCCTTT GGGCCGCCTC CCCGGCGGCC GCGCACCGTC GACTCGCTGA |
| 2161 | TCAGCCTCGA CTGTGCCTTC TAGTTGCCAG CCATCTGTTG TTTGCCCCTC CCCCGTGCCT |
| 2221 | TCCTTGACCC TGGAAGGTGC CACTCCCACT GTCCTTTCCT AATAAAATGA GGAAATTGCA |
| 2281 | TCGCATTGTC TGAGTAGGTG TCATTCTATT CTGGGGGGTG GGGTGGGGCA GGACAGCAAG |
| 2341 | GGGGAGGATT GGGAAGACAA TAGCAGGCAT GCTGGGGATG CGGTGGGCTC TATGGCTTCT |
| 2401 | GAGGCGGAAA GAACCAGCTG GGGCTCGACT AGAGCATGGC TACGTAGATA AGTAGCATGG |
| 2461 | CGGGTTAATC ATTAACTACA AGGAACCCCT AGTGATGGAG TTGGCCACTC CCTCTCTGCG |
| 2521 | CGCTCGCTCG CTCACTGAGG CCGGGCGACC AAAGGTCGCC CGACGCCCGG GCGGCCTCAG |
| 2581 | TGAGCGAGCG AGCGCGCAGA GCTTTTTGCA AAAGCCTAGG CCTCCAAAAA AGCCTCCTCA |
| 2641 | CTACTTCTGG AATAGCTCAG AGGCCGAGGC GGCCTCGGCC TCTGCATAAA TAAAAAAAAT |
| 2701 | TAGTCAGCCA TGGGGCGGAG AATGGGCGGA ACTGGGCGGA GTTAGGGGCG GGATGGGCGG |
| 2761 | AGTTAGGGGC GGGACTATGG TTGCTGACTA ATTGAGATGC ATGCTTTGCA TACTTCTGCC |
| 2821 | TGCTGGGGAG CCTGGGGACT TTCCACACCT GGTTGCTGAC TAATTGAGAT GCATGCTTTG |
| 2881 | CATACTTCTG CCTGCTGGGG AGCCTGGGGA CTTTCCACAC CCTAACTGAC ACACATTCCA |
| 2941 | CAGCTGCATT AATGAATCGG CCAACGCGCG GGGAGAGGCG GTTTGCGTAT TGGGCGCTCT |
| 3001 | TCCGCTTCCT CGCTCACTGA CTCGCTGCGC TCGGTCGTTC GGCTGCGGCG AGCGGTATCA |
| 3061 | GCTCACTCAA AGGCGGTAAT ACGGTTATCC ACAGAATCAG GGGATAACGC AGGAAAGAAC |
| 3121 | ATGTGAGCAA AAGGCCAGCA AAAGGCCAGG AACCGTAAAA AGGCCGCGTT GCTGGCGTTT |
| 3181 | TTCCATAGGC TCCGCCCCCC TGACGAGCAT CACAAAAATC GACGCTCAAG TCAGAGGTGG |
| 3241 | CGAAACCCGA CAGGACTATA AAGATACCAG GCGTTTCCCC CTGGAAGCTC CCTCGTGCGC |
| 3301 | TCTCCTGTTC CGACCCTGCC GCTTACCGGA TACCTGTCCG CCTTTCTCCC TTCGGGAAGC |
| 3361 | GTGGCGCTTT CTCATAGCTC ACGCTGTAGG TATCTCAGTT CGGTGTAGGT CGTTCGCTCC |
| 3421 | AAGCTGGGCT GTGTGCACGA ACCCCCCGTT CAGCCCGACC GCTGCGCCTT ATCCGGTAAC |
| 3481 | TATCGTCTTG AGTCCAACCC GGTAAGACAC GACTTATCGC CACTGGCAGC AGCCACTGGT |
| 3541 | AACAGGATTA GCAGAGCGAG GTATGTAGGC GGTGCTACAG AGTTCTTGAA GTGGTGGCCT |
| 3601 | AACTACGGCT ACACTAGAAG AACAGTATTT GGTATCTGCG CTCTGCTGAA GCCAGTTACC |
| 3661 | TTCGGAAAAA GAGTTGGTAG CTCTTGATCC GGCAAACAAA CCACCGCTGG TAGCGGTGGT |
| 3721 | TTTTTTGTTT GCAAGCAGCA GATTACGCGC AGAAAAAAAG GATCTCAAGA AGATCCTTTG |
| 3781 | ATCTTTTCTA CGGGGTCTGA CGCTCAGTGG AACGAAAACT CACGTTAAGG GATTTTGGTC |
| 3841 | ATGAGATTAT CAAAAAGGAT CTTCACCTAG ATCCTTTTAA ATTAAAAATG AAGTTTTAAA |
| 3901 | TCAATCTAAA GTATATATGA GTAAACTTGG TCTGACAGTT ACCAATGCTT AATCAGTGAG |
| 3961 | GCACCTATCT CAGCGATCTG TCTATTTCGT TCATCCATAG TTGCCTGACT CCTGCAAACC |
| 4021 | ACGTTGTGTC TCAAAATCTC TGATGTTACA TTGCACAAGA TAAAAATATA TCATCATGAA |
| 4081 | CAATAAAACT GTCTGCTTAC ATAAACAGTA ATACAAGGGG TGTTATGAGC CATATTCAAC |
| 4141 | GGGAAACGTC TTGCTCGAGG CCGCGATTAA ATTCCAACAT GGATGCTGAT TTATATGGGT |
| 4201 | ATAAATGGGC TCGCGATAAT GTCGGGCAAT CAGGTGCGAC AATCTATCGA TTGTATGGGA |
| 4261 | AGCCCGATGC GCCAGAGTTG TTTCTGAAAC ATGGCAAAGG TAGCGTTGCC AATGATGTTA |
| 4321 | CAGATGAGAT GGTCAGACTA AACTGGCTGA CGGAATTTAT GCCTCTTCCG ACCATCAAGC |
| 4381 | ATTTTATCCG TACTCCTGAT GATGCATGGT TACTCACCAC TGCGATCCCC GGGAAAACAG |
| 4441 | CATTCCAGGT ATTAGAAGAA TATCCTGATT CAGGTGAAAA TATTGTTGAT GCGCTGGCAG |
| 4501 | TGTTCCTGCG CCGGTTGCAT TCGATTCCTG TTTGTAATTG TCCTTTTAAC AGCGATCGCG |
| 4561 | TATTTCGTCT CGCTCAGGCG CAATCACGAA TGAATAACGG TTTGGTTGAT GCGAGTGATT |
| 4621 | TTGATGACGA GCGTAATGGC TGGCCTGTTG AACAAGTCTG GAAAGAAATG CATAAGCTTT |
| 4681 | TGCCATTCTC ACCGGATTCA GTCGTCACTC ATGGTGATTT CTCACTTGAT AACCTTATTT |
| 4741 | TTGACGAGGG GAAATTAATA GGTTGTATTG ATGTTGGACG AGTCGGAATC GCAGACCGAT |
| 4801 | ACCAGGATCT TGCCATCCTA TGGAACTGCC TCGGTGAGTT TTCTCCTTCA TTACAGAAAC |
| 4861 | GGCTTTTTCA AAAATATGGT ATTGATAATC CTGATATGAA TAAATTGCAG TTTCATTTGA |
| 4921 | TGCTCGATGA GTTTTTCTAA GGGCGGCCTG CCACCATACC CACGCCGAAA CAAGCGCTCA |
| 4981 | TGAGCCCGAA GTGGCGAGCC CGATCTTCCC CATCGGTGAT GTCGGCGATA TAGGCGCCAG |
| 5041 | CAACCGCACC TGTGGCGCCG GTGATGCCGG CCACGATGCG TCCGGCGTAG AGGATCTGGC |
| 5101 | TAGCGATGAC CCTGCTGATT GGTTCGCTGA CCATTTCCGG GTGCGGGACG GCGTTACCAG |
| 5161 | AAACTCAGAA GGTTCGTCCA ACCAAACCGA CTCTGACGGC AGTTTACGAG AGAGATGATA |
| 5221 | GGGTCTGCTT CAGGGTGACC GATGTAACCA TATACTTAGG CTGGATCTTC TCCCGCGAAT |
| 5281 | TTTAACCCTC ACCAACTACG AGATATGAGG TAAGCCAAAA AAGCACGTAG TGGCGCTCTC |
| 5341 | CGACTGTTCC CAAATTGTAA CTTATCGTTC CGTGAAGGCC AGAGTTACTT CCCGGCCCTT |
| 5401 | TCCATGCGCG CACCATACCC TCCTAGTTCC CCGGTTATCT TTCCGAAGTG GGAGTGAGCG |
| 5461 | AACCTCCGTT TACGTCTTGT TACCAATGAT GTAGCTATGC ACTTTGTACA GGGTGCCAAC |
| 5521 | GGGTTTCACA ATTCACAGAT AGTGGGGATC CCGGCAAAGG GCCTATATTT GCGGTCCAAC |
| 5581 | TTAGGCGTAA ACCTCGATGC TACCTACTCA GACCCACCTC GCGCGGGGTA AATAAGGCAC |
| 5641 | TCATCCCAGC TGGTTCTTGG CGTTCTACGC AGCGACATGT TTATTAACAG TTGTCTGGCA |
| 5701 | GCACAAAACT TTTACCATGG TCGTAGAAGC CCCCCAGAGT TAGTTCATAC CTAATGCCAC |
| 5761 | AAATGTGACA GGACGCCGAT GGGTACCGGA CTTTAGGTCG AGCACAGTTC GGTAACGGAG |
| 5821 | AGACCCTGCG GCGTACTTCA TTATGTATAT GGAACGTGCC CAAGTGACGC CAGGCAAGTC |
| 5881 | TCAGCTGGTT CCTGTGTTAG CTCGAGGGTA GACATACGAG CTGATTGAAC ATGGGTTGGG |
| 5941 | GGCCTCGAAC CGTCGAGGAC CCCATAGTAC CTCGGAGACC AAGTAGGGCA GCCTATAGTT |
| 6001 | TGAAGCAGAA CTATTTCGGG GGGCGAGCCC TCATCGTCTC TTCTGCGGAT GACTCAACAC |
| 6061 | GCTAGGGACG TGAAGTCGAT TCCTTCGATG GTTATAAATC AAAGACTCAG AGTGCTGTCT |
| 6121 | GGAGCGTGAA TCTAACGGTA CGTATCTCGA TTGCTCGGTC GCTTTTCGCA CTCCGCGAAA |
| 6181 | GTTCGTACCG CTCATTCACT AGGTTGCGAA GCCTATGCTG ATATATGAAT CCAAACTAGA |
| 6241 | GCAGGGCTCT TAAGATTCGG AGTTGTAAAT ACTTAATACT CCAATCGGCT TTTACGTGCA |
| 6301 | CCACCGCGGG CGGCTGACAA GGGTCTCACA TCGAGAAACA AGACAGTTCC GGGCTGGAAG |
| 6361 | TAGCGCCGGC TAAGGAAGAC GCCTGGTACG GCAGGACTAT GAAACCAGTA CAAAGGCAAC |
| 6421 | ATCCTCACTT GGGTGAACGG AAACGCAGTA TTATGGTTAC TTTTTGGATA CGTGAAACAT |
| 6481 | ATCCCATGGT AGTCCTTAGA CTTGGGAGTC TATCACCCCT AGGGCCCATA TCTGGAAATA |
| 6541 | GACGCCAGGT TGAATCCGTA TTTGGAGGTA CGATGGAACA GTCTGGGTGG GACGTGCTTC |
| 6601 | ATTTATACCC TGCGCAGGCT GGACCGAGGA CCGCAAGGTG CGGCGGTGCA CAAGCAATTG |
| 6661 | ACAACTAACC ACCGTGTATT CATTATGGTA CCAGGAACTT TAAGCCGAGT CAATGAAGCT |
| 6721 | CGCATTACAG TGTTTACCGC ATCTTGCCGT TACTCACAAA CTGTGATCCA CCACAAGTCA |
| 6781 | AGCCATTGCC TCTCTGACAC GCCGTAAGAA TTAATATGTA AACTTTGCGC GGGTTGACTG |
| 6841 | CGATCCGTTC AGTCTCGTCC GAGGGCACAA TCCTATTCCC ATTTGTATGT TCAGCTAACT |
| 6901 | TCTACCCATC CCCCGAAGTT AAGTAGGTCG TGAGATGCCA TGGAGGCTCT CGTTCATCCC |
| 6961 | GTGGGACATC AAGCTTCCCC TTGATAAAGC ACCCCGCTCG GGTGTAGCAG AGAAGACGCC |
| 7021 | TTCTGAATTG TGCAATCCCT CCACCTTATC TAAGCTTGCT ACCAATAATT AGCATTTTTG |
| 7081 | CCTTGCGACA GACCTCCTAC TTAGATTGCC ACACATTGAG CTAGTCAGTG AGCGATAAGC |
| 7141 | TTGACGCGCT TTCAAGGGTC GCGAGTACGT GAACTAAGGC TCCGGACAGG ACTATATACT |
| 7201 | TGGGTTTGAT CTCGCCCCGA CAACTGCAAA CCTCAACTTT TTTAGATTAT ATGGTTAGCC |
| 7261 | GAAGTTGCAC GAGGTGGCGT CCGCGGACTG CTCCCCGAGT GTGGCTCTTT CATCTGACAA |
| 7321 | CGTGCAACCC CTATCGCGGC CGATTGTTTC TGCGGACGAT GTTGTCCTCA TAGTTTGGGC |
| 7381 | ATGTTTCCCT TGTAGGTGTG AAACCACTTA GCTTCGCGCC GTAGTCCCAA TGAAAAACCT |
| 7441 | ATGGACTTTG TTTTGGGTAG CACCAGGAAT CTGAACCGTG TGAATGTGGA CGTCGCGCGC |
| 7501 | GTAGACCTTT ATCTCCGGTT CAAGCTAGGG ATGTGGCTGC ATGCTACGTT GTCACACCTA |
| 7561 | CACTGCTCGA AGTAAATATG CGAAGCGCGC GGCCTGGCCG GAGGCGTTCC GCGCCGCCAC |
| 7621 | GTGTTCGTTA ACTGTTGATT GGTGGCACAT AAGCAATATC GTAGTCCGTC AAATTCAGCT |
| 7681 | CTGTTATCCC GGGCGTTATG TGTCAAATGG CGTAGAACGG GATTGACTGT TTGACGGTAG |
| 7741 | GGTGACCTAA GCCAGATGCT ACACAATTAG GCTTGTACAT ATTGTCGTTA GAACGCGGCT |
| 7801 | ACAATTAATA CATAACCTTA TGTATCATAC ACATACGATT TAGGTGACAC TATAGAATAC |
| 7861 | ACGGAATTAA TTC. |

TABLE 4
Features of the VMD2.GFP.WPRE.pA plasmid sequence

| | | Mini- | Maxi- | | Direc- |
|---|---|---|---|---|---|
| Name | Type | mum | mum | Length | tion |
| Randomly | Stuffer | 5,241 | 7,740 | 2,500 | none |
| generated | | | | | |
| stuffer sequence | | | | | |
| AphR (KanR) | CDS | 4,125 | 4,940 | 816 | forward |
| pBR322 rep | rep_origin | 3,165 | 3,784 | 620 | reverse |
| origin | | | | | |
| AAV2 ITR | LTR | 2,481 | 2,601 | 121 | reverse |
| bGH pA | polyA_signal | 2,155 | 2,423 | 269 | forward |
| WPRE | WPRE | 1,547 | 2,136 | 590 | forward |
| GFP | misc_feature | 818 | 1,534 | 717 | forward |
| Kozak | Kozak | 812 | 817 | 6 | forward |
| −585 to +38 | promoter | 189 | 811 | 623 | forward |
| VMD2 promoter | | | | | |
| AAV2 ITR | LTR | 4 | 133 | 130 | forward |

An exemplary a vector of the disclosure comprising a marker includes VMD.IntEx.GFP.WPRE.pA. A nucleic acid sequence encoding a VMD.IntEx.GFP.WPRE.pA construct comprises or consists of:

| (SEQ ID NO: 17) | |
|---|---|
| 1 | TAGCTGCGCG CTCGCTCGCT CACTGAGGCC GCCCGGGCAA AGCCCGGGCG TCGGGCGACC |
| 61 | TTTGGTCGCC CGGCCTCAGT GAGCGAGCGA GCGCGCAGAG AGGGAGTGGC CAACTCCATC |
| 121 | ACTAGGGGTT CCTTGTAGTT AATGATTAAC CCGCCATGCT ACTTATCTAC GTAGCCATGC |
| 181 | TCTAGGTAAA TTCTGTCATT TTACTAGGGT GATGAAATTC CCAAGCAACA CCATCCTTTT |
| 241 | CAGATAAGGG CACTGAGGCT GAGAGAGGAG CTGAAACCTA CCCGGGGTCA CCACACACAG |
| 301 | GTGGCAAGGC TGGGACCAGA AACCAGGACT GTTGACTGCA GCCCGGTATT CATTCTTTCC |
| 361 | ATAGCCCACA GGGCTGTCAA AGACCCCAGG GCCTAGTCAG AGGCTCCTCC TTCCTGGAGA |
| 421 | GTTCCTGGCA CAGAAGTTGA AGCTCAGCAC AGCCCCCTAA CCCCCAACTC TCTCTGCAAG |
| 481 | GCCTCAGGGG TCAGAACACT GGTGGAGCAG ATCCTTTAGC CTCTGGATTT TAGGGCCATG |
| 541 | GTAGAGGGGG TGTTGCCCTA AATTCCAGCC CTGGTCTCAG CCCAACACCC TCCAAGAAGA |
| 601 | AATTAGAGGG GCCATGGCCA GGCTGTGCTA GCCGTTGCTT CTGAGCAGAT TACAAGAAGG |
| 661 | GACTAAGACA AGGACTCCTT TGTGGAGGTC CTGGCTTAGG GAGTCAAGTG ACGGCGGCTC |
| 721 | AGCACTCACG TGGGCAGTGC CAGCCTCTAA GAGTGGGCAG GGGCACTGGC CACAGAGTCC |
| 781 | CAGGGAGTCC CACCAGCCTA GTCGCCAGAC CGGGTGCCGC AGGGGGACGG CTGCCTTCGG |
| 841 | GGGGGACGGG GCAGGGCGGG GTTCGGCTTC TGGCGTGTGA CCGGCGGCTC TAGAGCCTCT |
| 901 | GCTAACCATG TTCATGCCTT CTTCTTTTTC CTACAGCTCC TGGGCAACGT GCTGGTTATT |
| 961 | GTGCTGTCTC ATCATTTTGG CAAAGAATTG GCACCATGAG CAAGGGCGAG GAACTGTTCA |
| 1021 | CTGGCGTGGT CCCAATTCTC GTGGAACTGG ATGGCGATGT GAATGGGCAC AAATTTTCTG |
| 1081 | TCAGCGGAGA GGGTGAAGGT GATGCCACAT ACGGAAAGCT CACCCTGAAA TTCATCTGCA |
| 1141 | CCACTGGAAA GCTCCCTGTG CCATGGCCAA CACTGGTCAC TACCCTGACC TATGGCGTGC |
| 1201 | AGTGCTTTTC CAGATACCCA GACCATATGA AGCAGCATGA CTTTTTCAAG AGCGCCATGC |
| 1261 | CCGAGGGCTA TGTGCAGGAG AGAACCATCT TTTTCAAAGA TGACGGGAAC TACAAGACCC |
| 1321 | GCGCTGAAGT CAAGTTCGAA GGTGACACCC TGGTGAATAG AATCGAGCTG AAGGGCATTG |
| 1381 | ACTTTAAGGA GGATGGAAAC ATTCTCGGCC ACAAGCTGGA ATACAACTAT AACTCCCACA |
| 1441 | ATGTGTACAT CATGGCCGAC AAGCAAAAGA ATGGCATCAA GGTCAACTTC AAGATCAGAC |
| 1501 | ACAACATTGA GGATGGATCC GTGCAGCTGG CCGACCATTA TCAACAGAAC ACTCCAATCG |
| 1561 | GCGACGGCCC TGTGCTCCTC CCAGACAACC ATTACCTGTC CACCCAGTCT GCCCTGTCTA |
| 1621 | AAGATCCCAA CGAAAAGAGA GACCACATGG TCCTGCTGGA GTTTGTGACC GCTGCTGGGA |
| 1681 | TCACACATGG CATGGACGAG CTGTACAAGT GAAAGCTTAT CGATAATCAA CCTCTGGATT |
| 1741 | ACAAAATTTG TGAAAGATTG ACTGGTATTC TTAACTATGT TGCTCCTTTT ACGCTATGTG |
| 1801 | GATACGCTGC TTTAATGCCT TTGTATCATG CTATTGCTTC CCGTATGGCT TTCATTTTCT |
| 1861 | CCTCCTTGTA TAAATCCTGG TTGCTGTCTC TTTATGAGGA GTTGTGGCCC GTTGTCAGGC |
| 1921 | AACGTGGCGT GGTGTGCACT GTGTTTGCTG ACGCAACCCC CACTGGTTGG GGCATTGCCA |
| 1981 | CCACCTGTCA GCTCCTTTCC GGGACTTTCG CTTTCCCCCT CCCTATTGCC ACGGCGGAAC |
| 2041 | TCATCGCCGC CTGCCTTGCC CGCTGCTGGA CAGGGGCTCG GCTGTTGGGC ACTGACAATT |
| 2101 | CCGTGGTGTT GTCGGGGAAA TCATCGTCCT TTCCTTGGCT GCTCGCCTGT GTTGCCACCT |
| 2161 | GGATTCTGCG CGGGACGTCC TTCTGCTACG TCCCTTCGGC CCTCAATCCA GCGGACCTTC |
| 2221 | CTTCCCGCGG CCTGCTGCCG GCTCTGCGGC CTCTTCCGCG TCTTCGCCTT CGCCCTCAGA |
| 2281 | CGAGTCGGAT CTCCCTTTGG GCCGCCTCCC CGGCGGCCGC GCACCGTCGA CTCGCTGATC |
| 2341 | AGCCTCGACT GTGCCTTCTA GTTGCCAGCC ATCTGTTGTT TGCCCCTCCC CCGTGCCTTC |
| 2401 | CTTGACCCTG GAAGGTGCCA CTCCCACTGT CCTTTCCTAA TAAAATGAGG AAATTGCATC |
| 2461 | GCATTGTCTG AGTAGGTGTC ATTCTATTCT GGGGGGTGGG GTGGGGCAGG ACAGCAAGGG |
| 2521 | GGAGGATTGG GAAGACAATA GCAGGCATGC TGGGGATGCG GTGGGCTCTA TGGCTTCTGA |
| 2581 | GGCGGAAAGA ACCAGCTGGG GCTCGACTAG AGCATGGCTA CGTAGATAAG TAGCATGGCG |
| 2641 | GGTTAATCAT TAACTACAAG GAACCCCTAG TGATGGAGTT GGCCACTCCC TCTCTGCGCG |
| 2701 | CTCGCTCGCT CACTGAGGCC GGGCGACCAA AGGTCGCCCG ACGCCCGGGC GGCCTCAGTG |
| 2761 | AGCGAGCGAG CGCGCAGAGC TTTTTGCAAA AGCCTAGGCC TCCAAAAAAG CCTCCTCACT |
| 2821 | ACTTCTGGAA TAGCTCAGAG GCCGAGGCGG CCTCGGCCTC TGCATAAATA AAAAAAATTA |
| 2881 | GTCAGCCATG GGGCGGAGAA TGGGCGGAAC TGGGCGGAGT TAGGGGCGGG ATGGGCGGAG |
| 2941 | TTAGGGGCGG GACTATGGTT GCTGACTAAT TGAGATGCAT GCTTTGCATA CTTCTGCCTG |
| 3001 | CTGGGGAGCC TGGGGACTTT CCACACCTGG TTGCTGACTA ATTGAGATGC ATGCTTTGCA |
| 3061 | TACTTCTGCC TGCTGGGGAG CCTGGGGACT TTCCACACCC TAACTGACAC ACATTCCACA |
| 3121 | GCTGCATTAA TGAATCGGCC AACGCGCGGG GAGAGGCGGT TTGCGTATTG GGCGCTCTTC |
| 3181 | CGCTTCCTCG CTCACTGACT CGCTGCGCTC GGTCGTTCGG CTGCGGCGAG CGGTATCAGC |
| 3241 | TCACTCAAAG GCGGTAATAC GGTTATCCAC AGAATCAGGG GATAACGCAG GAAAGAACAT |
| 3301 | GTGAGCAAAA GGCCAGCAAA AGGCCAGGAA CCGTAAAAAG GCCGCGTTGC TGGCGTTTTT |
| 3361 | CCATAGGCTC CGCCCCCCTG ACGAGCATCA CAAAAATCGA CGCTCAAGTC AGAGGTGGCG |
| 3421 | AAACCCGACA GGACTATAAA GATACCAGGC GTTTCCCCCT GGAAGCTCCC TCGTGCGCTC |
| 3481 | TCCTGTTCCG ACCCTGCCGC TTACCGGATA CCTGTCCGCC TTTCTCCCTT CGGGAAGCGT |
| 3541 | GGCGCTTTCT CATAGCTCAC GCTGTAGGTA TCTCAGTTCG GTGTAGGTCG TTCGCTCCAA |
| 3601 | GCTGGGCTGT GTGCACGAAC CCCCCGTTCA GCCCGACCGC TGCGCCTTAT CCGGTAACTA |
| 3661 | TCGTCTTGAG TCCAACCCGG TAAGACACGA CTTATCGCCA CTGGCAGCAG CCACTGGTAA |
| 3721 | CAGGATTAGC AGAGCGAGGT ATGTAGGCGG TGCTACAGAG TTCTTGAAGT GGTGGCCTAA |
| 3781 | CTACGGCTAC ACTAGAAGAA CAGTATTTGG TATCTGCGCT CTGCTGAAGC CAGTTACCTT |
| 3841 | CGGAAAAAGA GTTGGTAGCT CTTGATCCGG CAAACAAACC ACCGCTGGTA GCGGTGGTTT |
| 3901 | TTTTGTTTGC AAGCAGCAGA TTACGCGCAG AAAAAAAGGA TCTCAAGAAG ATCCTTTGAT |
| 3961 | CTTTTCTACG GGGTCTGACG CTCAGTGGAA CGAAAACTCA CGTTAAGGGA TTTTGGTCAT |
| 4021 | GAGATTATCA AAAAGGATCT TCACCTAGAT CCTTTTAAAT TAAAAATGAA GTTTTAAATC |
| 4081 | AATCTAAAGT ATATATGAGT AAACTTGGTC TGACAGTTAC CAATGCTTAA TCAGTGAGGC |
| 4141 | ACCTATCTCA GCGATCTGTC TATTTCGTTC ATCCATAGTT GCCTGACTCC TGCAAACCAC |
| 4201 | GTTGTGTCTC AAAATCTCTG ATGTTACATT GCACAAGATA AAAATATATC ATCATGAACA |
| 4261 | ATAAAACTGT CTGCTTACAT AAACAGTAAT ACAAGGGGTG TTATGAGCCA TATTCAACGG |
| 4321 | GAAACGTCTT GCTCGAGGCC GCGATTAAAT TCCAACATGG ATGCTGATTT ATATGGGTAT |
| 4381 | AAATGGGCTC GCGATAATGT CGGGCAATCA GGTGCGACAA TCTATCGATT GTATGGGAAG |
| 4441 | CCCGATGCGC CAGAGTTGTT TCTGAAACAT GGCAAAGGTA GCGTTGCCAA TGATGTTACA |
| 4501 | GATGAGATGG TCAGACTAAA CTGGCTGACG GAATTTATGC CTCTTCCGAC CATCAAGCAT |
| 4561 | TTTATCCGTA CTCCTGATGA TGCATGGTTA CTCACCACTG CGATCCCCGG GAAAACAGCA |
| 4621 | TTCCAGGTAT TAGAAGAATA TCCTGATTCA GGTGAAAATA TTGTTGATGC GCTGGCAGTG |
| 4681 | TTCCTGCGCC GGTTGCATTC GATTCCTGTT TGTAATTGTC CTTTTAACAG CGATCGCGTA |
| 4741 | TTTCGTCTCG CTCAGGCGCA ATCACGAATG AATAACGGTT TGGTTGATGC GAGTGATTTT |
| 4801 | GATGACGAGC GTAATGGCTG GCCTGTTGAA CAAGTCTGGA AAGAAATGCA TAAGCTTTTG |
| 4861 | CCATTCTCAC CGGATTCAGT CGTCACTCAT GGTGATTTCT CACTTGATAA CCTTATTTTT |
| 4921 | GACGAGGGGA AATTAATAGG TTGTATTGAT GTTGGACGAG TCGGAATCGC AGACCGATAC |
| 4981 | CAGGATCTTG CCATCCTATG GAACTGCCTC GGTGAGTTTT CTCCTTCATT ACAGAAACGG |
| 5041 | CTTTTTCAAA AATATGGTAT TGATAATCCT GATATGAATA AATTGCAGTT TCATTTGATG |
| 5101 | CTCGATGAGT TTTTCTAAGG GCGGCCTGCC ACCATACCCA CGCCGAAACA AGCGCTCATG |
| 5161 | AGCCCGAAGT GGCGAGCCCG ATCTTCCCCA TCGGTGATGT CGGCGATATA GGCGCCAGCA |
| 5221 | ACCGCACCTG TGGCGCCGGT GATGCCGGCC ACGATGCGTC CGGCGTAGAG GATCTGGCTA |
| 5281 | GCGATGACCC TGCTGATTGG TTCGCTGACC ATTTCCGGGT GCGGGACGGC GTTACCAGAA |
| 5341 | ACTCAGAAGG TTCGTCCAAC CAAACCGACT CTGACGGCAG TTTACGAGAG AGATGATAGG |
| 5401 | GTCTGCTTCA GGGTGACCGA TGTAACCATA TACTTAGGCT GGATCTTCTC CCGCGAATTT |
| 5461 | TAACCCTCAC CAACTACGAG ATATGAGGTA AGCCAAAAAA GCACGTAGTG GCGCTCTCCG |
| 5521 | ACTGTTCCCA AATTGTAACT TATCGTTCCG TGAAGGCCAG AGTTACTTCC CGGCCCTTTC |
| 5581 | CATGCGCGCA CCATACCCTC CTAGTTCCCC GGTTATCTTT CCGAAGTGGG AGTGAGCGAA |
| 5641 | CCTCCGTTTA CGTCTTGTTA CCAATGATGT AGCTATGCAC TTTGTACAGG GTGCCAACGG |
| 5701 | GTTTCACAAT TCACAGATAG TGGGGATCCC GGCAAAGGGC CTATATTTGC GGTCCAACTT |
| 5761 | AGGCGTAAAC CTCGATGCTA CCTACTCAGA CCCACCTCGC GCGGGGTAAA TAAGGCACTC |
| 5821 | ATCCCAGCTG GTTCTTGGCG TTCTACGCAG CGACATGTTT ATTAACAGTT GTCTGGCAGC |
| 5881 | ACAAAACTTT TACCATGGTC GTAGAAGCCC CCCAGAGTTA GTTCATACCT AATGCCACAA |
| 5941 | ATGTGACAGG ACGCCGATGG GTACCGGACT TTAGGTCGAG CACAGTTCGG TAACGGAGAG |
| 6001 | ACCCTGCGGC GTACTTCATT ATGTATATGG AACGTGCCCA AGTGACGCCA GGCAAGTCTC |
| 6061 | AGCTGGTTCC TGTGTTAGCT CGAGGGTAGA CATACGAGCT GATTGAACAT GGGTTGGGGG |
| 6121 | CCTCGAACCG TCGAGGACCC CATAGTACCT CGGAGACCAA GTAGGGCAGC CTATAGTTTG |
| 6181 | AAGCAGAACT ATTTCGGGGG GCGAGCCCTC ATCGTCTCTT CTGCGGATGA CTCAACACGC |
| 6241 | TAGGGACGTG AAGTCGATTC CTTCGATGGT TATAAATCAA AGACTCAGAG TGCTGTCTGG |
| 6301 | AGCGTGAATC TAACGGTACG TATCTCGATT GCTCGGTCGC TTTTCGCACT CCGCGAAAGT |
| 6361 | TCGTACCGCT CATTCACTAG GTTGCGAAGC CTATGCTGAT ATATGAATCC AAACTAGAGC |
| 6421 | AGGGCTCTTA AGATTCGGAG TTGTAAATAC TTAATACTCC AATCGGCTTT TACGTGCACC |
| 6481 | ACCGCGGGCG GCTGACAAGG GTCTCACATC GAGAAACAAG ACAGTTCCGG GCTGGAAGTA |
| 6541 | GCGCCGGCTA AGGAAGACGC CTGGTACGGC AGGACTATGA AACCAGTACA AAGGCAACAT |
| 6601 | CCTCACTTGG GTGAACGGAA ACGCAGTATT ATGGTTACTT TTTGGATACG TGAAACATAT |
| 6661 | CCCATGGTAG TCCTTAGACT TGGGAGTCTA TCACCCCTAG GGCCCATATC TGGAAATAGA |
| 6721 | CGCCAGGTTG AATCCGTATT TGGAGGTACG ATGGAACAGT CTGGGTGGGA CGTGCTTCAT |
| 6781 | TTATACCCTG CGCAGGCTGG ACCGAGGACC GCAAGGTGCG GCGGTGCACA AGCAATTGAC |
| 6841 | AACTAACCAC CGTGTATTCA TTATGGTACC AGGAACTTTA AGCCGAGTCA ATGAAGCTCG |
| 6901 | CATTACAGTG TTTACCGCAT CTTGCCGTTA CTCACAAACT GTGATCCACC ACAAGTCAAG |
| 6961 | CCATTGCCTC TCTGACACGC CGTAAGAATT AATATGTAAA CTTTGCGCGG GTTGACTGCG |
| 7021 | ATCCGTTCAG TCTCGTCCGA GGGCACAATC CTATTCCCAT TTGTATGTTC AGCTAACTTC |
| 7081 | TACCCATCCC CCGAAGTTAA GTAGGTCGTG AGATGCCATG GAGGCTCTCG TTCATCCCGT |
| 7141 | GGGACATCAA GCTTCCCCTT GATAAAGCAC CCCGCTCGGG TGTAGCAGAG AAGACGCCTT |
| 7201 | CTGAATTGTG CAATCCCTCC ACCTTATCTA AGCTTGCTAC CAATAATTAG CATTTTTGCC |
| 7261 | TTGCGACAGA CCTCCTACTT AGATTGCCAC ACATTGAGCT AGTCAGTGAG CGATAAGCTT |
| 7321 | GACGCGCTTT CAAGGGTCGC GAGTACGTGA ACTAAGGCTC CGGACAGGAC TATATACTTG |
| 7381 | GGTTTGATCT CGCCCCGACA ACTGCAAACC TCAACTTTTT TAGATTATAT GGTTAGCCGA |
| 7441 | AGTTGCACGA GGTGGCGTCC GCGGACTGCT CCCCGAGTGT GGCTCTTTCA TCTGACAACG |
| 7501 | TGCAACCCCT ATCGCGGCCG ATTGTTTCTG CGGACGATGT TGTCCTCATA GTTTGGGCAT |
| 7561 | GTTTCCCTTG TAGGTGTGAA ACCACTTAGC TTCGCGCCGT AGTCCCAATG AAAAACCTAT |
| 7621 | GGACTTTGTT TTGGGTAGCA CCAGGAATCT GAACCGTGTG AATGTGGACG TCGCGCGCGT |
| 7681 | AGACCTTTAT CTCCGGTTCA AGCTAGGGAT GTGGCTGCAT GCTACGTTGT CACACCTACA |
| 7741 | CTGCTCGAAG TAAATATGCG AAGCGCGCGG CCTGGCCGGA GGCGTTCCGC GCCGCCACGT |
| 7801 | GTTCGTTAAC TGTTGATTGG TGGCACATAA GCAATATCGT AGTCCGTCAA ATTCAGCTCT |
| 7861 | GTTATCCCGG GCGTTATGTG TCAAATGGCG TAGAACGGGA TTGACTGTTT GACGGTAGGG |
| 7921 | TGACCTAAGC CAGATGCTAC ACAATTAGGC TTGTACATAT TGTCGTTAGA ACGCGGCTAC |
| 7981 | AATTAATACA TAACCTTATG TATCATACAC ATACGATTTA GGTGACACTA TAGAATACAC |
| 8041 | GGAATTAATT C. |

TABLE 5
Features of the VMD2.IntEx.GFP.WPRE.pA plasmid sequence

| | | Mini- | Maxi- | | Direc- |
|---|---|---|---|---|---|
| Name | Type | mum | mum | Length | tion |
| Randomly | Stuffer | 5,419 | 7,918 | 2,500 | none |
| generated | | | | | |
| stuffer sequence | | | | | |
| AphR (KanR) | CDS | 4,303 | 5,118 | 816 | forward |
| pBR322 rep | rep_origin | 3,343 | 3,962 | 620 | reverse |
| origin | | | | | |
| AAV2 ITR | LTR | 2,659 | 2,779 | 121 | reverse |
| bGH pA | polyA_signal | 2,333 | 2,601 | 269 | forward |
| WPRE | WPRE | 1,725 | 2,314 | 590 | forward |
| GFP | misc_feature | 996 | 1,712 | 717 | forward |
| Kozak | Kozak | 990 | 995 | 6 | forward |
| Exon | exon | 937 | 989 | 53 | forward |
| Intron | intron | 814 | 936 | 123 | forward |
| −585 to +38 | promoter | 189 | 811 | 623 | forward |
| VMD2 promoter | | | | | |
| AAV2 ITR | LTR | 4 | 133 | 130 | forward |

AAV particles

The AAV vectors of the disclosure contain an AAV genome that has been derivatized for the purpose of administration to patients. Such derivatization is standard in the art and the invention encompasses the use of any known derivative of an AAV genome, and derivatives which could be generated by applying techniques known in the art. Derivatization of the AAV genome and of the AAV capsid are reviewed in Coura and Nardi (2007) Virology Journal 4: 99, and in Choi et al. and Wu et al., referenced above.

Derivatives of an AAV genome include any truncated or modified forms of an AAV genome which allow for expression of a transgene from a vector of the invention in vivo. It is possible to truncate the AAV genome significantly to include minimal viral sequence yet retain the above function. This is preferred for safety reasons to reduce the risk of recombination of the vector with wild-type virus, and also to avoid triggering a cellular immune response by the presence of viral gene proteins in the target cell.

The following portions could therefore be removed in a derivative of the invention: one inverted terminal repeat (ITR) sequence, the replication (rep) and capsid (cap) genes. However, in some embodiments, derivatives may additionally include one or more rep and/or cap genes or other viral sequences of an AAV genome. Naturally occurring AAV integrates with a high frequency at a specific site on human chromosome 19, and shows a negligible frequency of random integration, such that retention of an integrative capacity in the vector may be tolerated in a therapeutic setting.

The AAV genome comprises packaging genes, such as rep and/or cap genes which encode packaging functions for an AAV particle. The rep gene encodes one or more of the proteins Rep78, Rep68, Rep52 and Rep40 or variants thereof. The cap gene encodes one or more capsid proteins such as VP1, VP2 and VP3 or variants thereof. These proteins make up the capsid of an AAV particle.

Where a derivative comprises capsid proteins i.e. VP1, VP2 and/or VP3, the derivative may be a chimeric, shuffled or capsid-modified derivative of one or more naturally occurring AAVs. In particular, the invention encompasses the provision of capsid protein sequences from different serotypes, clades, clones, or isolates of AAV within the same vector (i.e. a pseudotyped vector).

Chimeric, shuffled or capsid-modified derivatives are selected to provide one or more desired functionalities for the viral vector. Thus, these derivatives may display increased efficiency of gene delivery, decreased immunogenicity (humoral or cellular), an altered tropism range and/or improved targeting of a particular cell type compared to an AAV vector comprising a naturally occurring AAV genome, such as that of AAV2. Increased efficiency of gene delivery may be effected by improved receptor or co-receptor binding at the cell surface, improved internalization, improved trafficking within the cell and into the nucleus, improved uncoating of the viral particle and improved conversion of a single-stranded genome to double-stranded form. Increased efficiency may also relate to an altered tropism range or targeting of a specific cell population, such that the vector dose is not diluted by administration to tissues where it is not needed.

Chimeric capsid proteins include those generated by recombination between two or more capsid coding sequences of naturally occurring AAV serotypes. This may be performed for example by a marker rescue approach in which non-infectious capsid sequences of one serotype are co-transfected with capsid sequences of a different serotype, and directed selection is used to select for capsid sequences having desired properties. The capsid sequences of the different serotypes can be altered by homologous recombination within the cell to produce novel chimeric capsid proteins.

Chimeric capsid proteins also include those generated by engineering of capsid protein sequences to transfer specific capsid protein domains, surface loops or specific amino acid residues between two or more capsid proteins, for example between two or more capsid proteins of different serotypes.

Shuffled or chimeric capsid proteins may also be generated by DNA shuffling or by error-prone PCR. Hybrid AAV capsid genes can be created by randomly fragmenting the sequences of related AAV genes e.g. those encoding capsid proteins of multiple different serotypes and then subsequently reassembling the fragments in a self-priming polymerase reaction, which may also cause crossovers in regions of sequence homology. A library of hybrid AAV genes created in this way by shuffling the capsid genes of several serotypes can be screened to identify viral clones having a desired functionality. Similarly, error prone PCR may be used to randomly mutate AAV capsid genes to create a diverse library of variants which may then be selected for a desired property.

The sequences of the capsid genes may also be genetically modified to introduce specific deletions, substitutions or insertions with respect to the native wild-type sequence. In particular, capsid genes may be modified by the insertion of a sequence of an unrelated protein or peptide within an open reading frame of a capsid coding sequence, or at the N- and/or C-terminus of a capsid coding sequence. The unrelated protein or peptide may advantageously be one which acts as a ligand for a particular cell type, thereby conferring improved binding to a target cell or improving the specificity of targeting of the vector to a particular cell population. The unrelated protein may also be one which assists purification of the viral particle as part of the production process, i.e. an epitope or affinity tag. The site of insertion will is selected so as not to interfere with other functions of the viral particle e.g. internalization, trafficking of the viral particle. The skilled person can identify suitable sites for insertion based on their common general knowledge. Particular sites are disclosed in Choi et al., referenced above.

The invention additionally encompasses the provision of sequences of an AAV genome in a different order and configuration to that of a native AAV genome. The invention also encompasses the replacement of one or more AAV sequences or genes with sequences from another virus or with chimeric genes composed of sequences from more than one virus. Such chimeric genes may be composed of sequences from two or more related viral proteins of different viral species.

AAV vectors of the invention include transcapsidated forms wherein an AAV genome or derivative having an ITR of one serotype is packaged in the capsid of a different serotype. AAV vectors of the invention also include mosaic forms wherein a mixture of unmodified capsid proteins from two or more different serotypes makes up the viral capsid. An AAV vector may also include chemically modified forms bearing ligands adsorbed to the capsid surface. For example, such ligands may include antibodies for targeting a particular cell surface receptor.

Thus, for example, AAV vectors of the invention include those with an AAV2 genome and AAV2 capsid proteins (AAV2/2), those with an AAV2 genome and AAV5 capsid proteins (AAV2/5) and those with an AAV2 genome and AAV8 capsid proteins (AAV2/8). An AAV vector of the invention may comprise a mutant AAV capsid protein. In one embodiment, an AAV vector of the invention comprises a mutant AAV8 capsid protein. Preferably the mutant AAV8 capsid protein is an AAV8 Y733F capsid protein.

Methods of making AAV viral particles of the disclosure will be known to one of skill in the art. An exemplary, but non-limiting method of preparing AAV viral particles of the disclosure is described below. For generation of a given AAV vector, three plasmids are required: one comprising the viral delivery vector encoding the nucleic acid sequence of interest to be delivered (i.e the nucleic acid sequence encoding BEST1), a plasmid encoding the rep and cap genes, and a third helper plasmid that contains the required adenoviral genes necessary for successful AAV generation. A promoter may be operably linked to each of the packaging genes. Specific examples of such promoters include the p5, p19 and p40 promoters (Laughlin et al. (1979) Proc. Natl. Acad. Sci. USA 76: 5567-5571). For example, the p5 and p19 promoters are generally used to express the rep gene, while the p40 promoter is generally used to express the cap gene. The plasmids are used to transfect suitable cells that are capable of replicating the AAV viral vector, transcribing and translating the AAV protein, and packaging the AAV viral vector into an AAV viral particle. Exemplary suitable cells comprise HEK293 cells. Post-transfection, the cells are collected and lysed. AAV particles can then be purified from the lysate through a variety of methods. Alternatively, AAV particles can be purified from the supernatant. For example, the lysate can be treated with Benzonase and clarified before applying to an iodixanol gradient comprised of 15%, 25%, 40% and 60% phases. The gradients can spun at 59,000 rpm for 1 hour 30 minutes and the 40% fraction then withdrawn. This AAV phase can then purified and concentrated using an Amicon Ultra-15 100K filter unit.

### Pharmaceutical Compositions

The AAV vectors of the invention may be formulated into pharmaceutical compositions. These compositions may comprise, in addition to the medicament, a pharmaceutically acceptable carrier, diluent, excipient, buffer, stabilizer or other materials well known in the art. Such materials should be non-toxic and should not interfere with the efficacy of the active ingredient. The precise nature of the carrier or other material may be determined by the skilled person according to the route of administration, e.g. subretinal, direct retinal or intravitreal injection.

The pharmaceutical composition may be formulated as a liquid. Liquid pharmaceutical compositions may include a liquid carrier such as water, petroleum, animal or vegetable oils, mineral oil or synthetic oil. Physiological saline solution, magnesium chloride, dextrose or other saccharide solution, or glycols such as ethylene glycol, propylene glycol or polyethylene glycol may be included. In some cases, a surfactant, such as pluronic acid (PF68) 0.001% may be used.

For injection at the site of affliction, the active ingredient may be in the form of an aqueous solution which is pyrogen-free, and has suitable pH, isotonicity and stability. The skilled person is well able to prepare suitable solutions using, for example, isotonic vehicles such as Sodium Chloride Injection, Ringer's Injection or Lactated Ringer's Injection. Preservatives, stabilisers, buffers, antioxidants and/or other additives may be included as required.

Buffers may have an effect on the stability and biocompatibity of the viral vectors and vector particles of the disclosure following storage and passage through injection devices for AAV gene therapy. In some embodiments, the viral vectors and vector particles of the disclosure may be diluted in TMN 200 buffer to maintain biocompatibility and stability. TMN 200 buffer comprises 20 mM Tris (pH adjusted to 8.0), 1 mM MgCl₂ and 200 mM NaCl.

The determination of the physical viral genome titer comprises part of the characterization of the viral vector or viral particle. In some embodiments, determination of the physical viral genome titre comprises a step in ensuring the potency and safety of viral vectors and viral particles during gene therapy. In some embodiments, a method to determine the AAV titer comprises quantitative PCR (qPCR). There are different variables that can influence the results, such as the conformation of the DNA used as standard or the enzymatic digestion during the sample preparation. The viral vector or particle preparation whose titer may be measured may be compared against a standard dilution curve generated using a plasmid. In some embodiments, the plasmid DNA used in the standard curve is in the supercoiled conformation. In some embodiments, the plasmid DNA used in the standard curve is in the linear conformation. Linearized plasmid can be prepared, for example by digestion with HindIII restriction enzyme, visualized by agarose gel electrophoresis and purified using the QIAquick Gel Extraction Kit (Qiagen) following manufacturer's instructions. Other restriction enzymes that cut within the plasmid used to generate the standard curve may also be appropriate. In some embodiments, the use of supercoiled plasmid as the standard increased the titre of the AAV vector compared to the use of linearized plasmid.

To extract the DNA from purified AAV vectors for quantification of AAV genome titer, two enzymatic methods can be used. In some embodiments, the AAV vector may be singly digested with DNase I. In some embodiments, the AAV vector may be double digested with DNase I and an additional proteinase K treatment. QPCR can then performed with the CFX Connect Real-Time PCR Detection System (BioRad) using primers and Taqman probe specific to the transgene sequence.

For delayed release, the medicament may be included in a pharmaceutical composition which is formulated for slow release, such as in microcapsules formed from biocompatible polymers or in liposomal carrier systems according to methods known in the art.

### Dosages

As used herein, the term “Dnase resistant particle (DRP)” refers to AAV particles that are resistant to Dnase digestion, and are therefore thought to completely encapsulate and protect the AAV vector of the disclosure from Dnase digestion. AAV particles may also be quantified in terms of the total numbers of genome particles (gp) administered in a dose, or gp/mL, the number genome particles per milliliter (mL) of solution. As used herein, genome particle (gp) refers to AAV particles containing a copy of an AAV delivery vector (or AAV genome) of the disclosure. As used herein, the term genome content (GC) per mL refers to the number of viral genomes per mL of solution, and may be determined, for example, by qPCR as described above. The terms GC and VG (viral genomes) may be used synonymously to characterize AAV dosages and concentrations of the disclosure.

In some embodiments of the compositions of the disclosure, a composition comprising an AAV vector or an AAV vector is administered to a subject as a single dose.

In some embodiments of the compositions of the disclosure, a composition comprising an AAV vector or an AAV vector may be formulated as a liquid suspension wherein the AAV vectors are suspended in a pharmaceutically-acceptable carrier. In some embodiments, compositions of the disclosure may comprise a plurality of AAV vectors at a concentration of 1-2×10⁹, 1-2×10¹⁰, 1-2×10¹¹, 1-2×10¹² or 1-2×10¹³ genome particles (gp) per mL. In some embodiments, compositions of the disclosure may comprise a plurality of AAV vectors at a concentration of 5×10¹¹ DRP/mL, 1.5×10¹² DRP/mL, 5×10¹² DRP/mL, 1.2×10¹² DRP/mL, 4.5×10¹² DRP/mL, 1.2×10¹³ DRP/mL, 1.5×10¹³ DRP/mL or 5×10¹³ DRP/1.2×10¹² DRP/mL. In some embodiments, compositions of the disclosure may comprise a plurality of AAV vectors at a concentration of 5×10¹² DRP per mL. In some embodiments, compositions of the disclosure may comprise a plurality of AAV vectors at a concentration of 1.5×10¹³ DRP per mL. Thus, to administer a dose of AAV vector of about 2×10¹⁰ gp, for example, a single injection of about 10 microliters of a pharmaceutical composition having a concentration of about 2×10¹² gp per mL will achieve the desired dose in vivo.

In some embodiments of the compositions of the disclosure, a composition comprising an AAV vector or an AAV vector may comprise a volume of between 1 and 500 μl, inclusive of the endpoints. In some embodiments of the compositions of the disclosure, a composition comprising an AAV vector or an AAV vector may comprise a volume of between 10-500, 50-500, 100-500, 200-500, 300-500, 400-500, 50-250, 100-250, 200-250, 50-150, 1-100 or 1-10 μl, inclusive of the endpoints for each range. In some embodiments of the compositions of the disclosure, a composition comprising an AAV vector or an AAV vector may comprise a volume of 1, 2, 5, 10, 50, 100, 150, 200, 250, 300, 350, 400, 450, 500 μl or any number of microliters in between. In some embodiments, a composition comprising an AAV vector or an AAV vector may comprise 100 μl.

In some embodiments of the compositions of the disclosure, an entire volume of a composition comprising an AAV vector or an AAV vector may be injected in a single injection. In some embodiments, a portion of a volume of a composition comprising an AAV vector or an AAV vector may be injected in a single injection. In some embodiments, a first portion of a volume of a composition comprising an AAV vector or an AAV vector may be injected in a first single injection and a second portion of a volume of a composition comprising an AAV vector or an AAV vector may be injected in a second single injection

In some embodiments of the compositions of the disclosure, a composition comprising an AAV vector or an AAV vector is administered at a dosage of at least 2×10⁷, 2×10⁸, 5×10⁸, 1.5×10⁹, 2×10⁹, 5×10⁹, 2×10¹⁰, 5×10¹⁰, 6×10¹⁰, 1.2×10¹¹, 2×10¹¹, 4.5×10¹¹, 5×10¹¹, 1.2×10¹², 1.5×10¹², 2×10¹² or 5×10¹² gp per eye. In some embodiments, a composition comprising an AAV vector or an AAV vector is administered at a dosage of about 5×10¹⁰, 1.5×10¹¹, 5×10¹¹ or 1.5×10¹¹ gp per eye. In some embodiments, a composition comprising an AAV vector or an AAV vector is administered at a dosage of about 5×10¹¹ DRP per eye, by subretinal injection. In some embodiments, a composition comprising an AAV vector or an AAV vector is administered at a dosage of about 2×10¹⁰ gp per eye, by subretinal injection. In some embodiments, a composition comprising an AAV vector or an AAV vector is administered at a dosage of about 5×10¹⁰ gp per eye, by subretinal injection. In some embodiments, the AAV vector is administered at a dosage of about 6×10¹⁰ gp per eye, by subretinal injection. In some embodiments, a composition comprising an AAV vector or an AAV vector is administered at a dosage of about 1.5×10¹¹ gp per eye, by subretinal injection. In some embodiments, a composition comprising an AAV vector or an AAV vector is administered at a dosage of about 2×10¹¹ gp per eye, by subretinal injection. In some embodiments, a composition comprising an AAV vector or an AAV vector is administered at a dosage of about 5×10¹¹ gp per eye, by subretinal injection. In some embodiments, a composition comprising an AAV vector or an AAV vector is administered at a dosage of about 1.5×10¹² gp per eye, by subretinal injection.

Dosages or volumes may be calculated based on allometric scaling between species based on vitreal volume. “Allometry”, as used herein, refers to the changes in organisms with respect to body size. Some factors to take into account when comparing species include body volume, surface area, metabolic rate, and unique anatomical, physiological or biochemical processes. The human equivalent dose can be normalized to body surface area, body weight or a combination of surface area and weight. Other factors may also be taken into account.

### Delivery

The viral vectors of the invention may be administered to the eye of a subject by subretinal, direct retinal, suprachoroidal or intravitreal injection. A skilled person will be familiar with and well able to carry out individual subretinal, direct retinal or intravitreal injections.

Subretinal injections are injections into the subretinal space, i.e. underneath the neurosensory retina. During a subretinal injection, the injected material is directed into, and creates a space between, the photoreceptor cell and retinal pigment epithelial (RPE) layers. When the injection is carried out through a small retinotomy, a retinal detachment may be created. The detached, raised layer of the retina that is generated by the injected material is referred to as a “bleb”. The hole created by the subretinal injection must be sufficiently small that the injected solution does not significantly reflux back into the vitreous cavity after administration. Such reflux would be particularly problematic when a medicament is injected, because the effects of the medicament would be directed away from the target zone. Preferably, the injection creates a self-sealing entry point in the neurosensory retina, i.e. once the injection needle is removed, the hole created by the needle reseals such that very little or substantially no injected material is released through the hole.

To facilitate this process, specialist subretinal injection needles are commercially available (e.g. DORC 41G Teflon subretinal injection needle, Dutch Ophthalmic Research Center International BV, Zuidland, The Netherlands). These are needles designed to carry out subretinal injections.

Alternatively, subretinal injections can be performed by delivering the composition comprising AAV particles under direct visual guidance using an operating microscope (Leica Microsystems, Germany). One exemplary approach is that of using a scleral tunnel approach through the posterior pole to the superior retina with a Hamilton syringe and 34-gauge needle (ESS labs, UK). Alternatively, sub-retinal injections can be performed using an anterior chamber paracentesis with a 33G needle prior to the subretinal injection using a WPI syringe and a beveled 35G-needle system (World Precision Instruments, UK). An additional alternative is a WPI Nanofil Syringe (WPI, part #NANOFIL) and a 34 gauge WBI Nanofil needle (WPI, part # NF34BL-2).

Vectors or compositions of the disclosure may be administered via suprachoroidal injection. Any means of suprachoroidal injection is envisaged as a potential delivery system for a vector or a composition of the disclosure. Suprachoroidal injections are injections into the suprachoroidal space, which is the space between the choroid and the sclera. Injection into the suprachoroidal space is thus a potential route of administration for the delivery of compositions to proximate eye structures such as the retina, retinal pigment epithelium (RPE) or macula. In some embodiments, injection into the suprachoroidal space is done in an anterior portion of the eye using a microneedle, microcannula, or microcatheter. An anterior portion of the eye may comprise or consist of an area anterior to the equator of the eye. The vector composition or AAV viral particles may diffuse posteriorly from an injection site via a suprachoroidal route. In some embodiments, the suprachoroidal space in the posterior eye is injected directly using a catheter system. In this embodiment, the suprachoroidal space may be catheterized via an incision in the pars plana. In some embodiments, an injection or an infusion via a suprachoroidal route traverses the choroid, Bruch's membrane and/or RPE layer to deliver a vector or a composition of the disclosure to a subretinal space. In some embodiments, including those in which a vector or a composition of the disclosure is delivered to a subretinal space via a suprachoroidal route, one or more injections is made into at least one of the sclera, the pars plana, the choroid, the Bruch's membrane, and the RPE layer. In some embodiments, including those in which a vector or a composition of the disclosure is delivered to a subretinal space via a suprachoroidal route, a two-step procedure is used to create a bleb in a suprachoroidal or a subretinal space prior to delivery of a vector or a composition of the disclosure.

In those embodiments where mice are injected, animals can be anaesthetized by intraperitoneal injection containing ketamine (40-80 mg/kg) and xylazine (1-10 mg/kg) and pupils fully dilated with tropicamide eye drops (Mydriaticum 1%, Bausch & Lomb, UK) and phenylephrine eye drops (phenylephrine hydrochloride 2.5%, Bausch & Lomb, UK). Proxymetacaine eye drops (proxymetacaine hydrochloride 0.5%, Bausch & Lomb, UK) can also applied prior to sub-retinal injection. Post-injection, chloramphenicol eye drops can applied (chloramphenicol 0.5%, Bausch & Lomb, UK) and anaesthesia reversed with atipamezole (2 mg/kg) and carbomer gel applied (Viscotears, Novartis, UK) to prevent cataract formation.

Unless damage to the retina occurs during the injection, and as long as a sufficiently small needle is used, substantially all injected material remains localized between the detached neurosensory retina and the RPE at the site of the localized retinal detachment (i.e. does not reflux into the vitreous cavity). Indeed, the typical persistence of the bleb over a short time frame indicates that there is usually little escape of the injected material into the vitreous. The bleb may dissipate over a longer time frame as the injected material is absorbed.

Visualizations of the eye, in particular the retina, for example using optical coherence tomography, may be made pre-operatively.

The AAV vectors of the invention may be delivered with increased accuracy and safety by using a two-step method in which a localized retinal detachment is created by the subretinal injection of a first solution. The first solution does not comprise the vector. A second subretinal injection is then used to deliver the medicament comprising the vector into the subretinal fluid of the bleb created by the first subretinal injection. Because the injection delivering the medicament is not being used to detach the retina, a specific volume of solution may be injected in this second step. An AAV vector of the invention may be delivered by: (a) administering a solution to the subject by subretinal injection in an amount effective to at least partially detach the retina to form a subretinal bleb, wherein the solution does not comprise the vector; and (b) administering a medicament composition by subretinal injection into the bleb formed by step (a), wherein the medicament comprises the vector.

### EXAMPLES

### Example 1: Bestrophin-1 Protein in HEK293 Cells Using the CAG Promoter

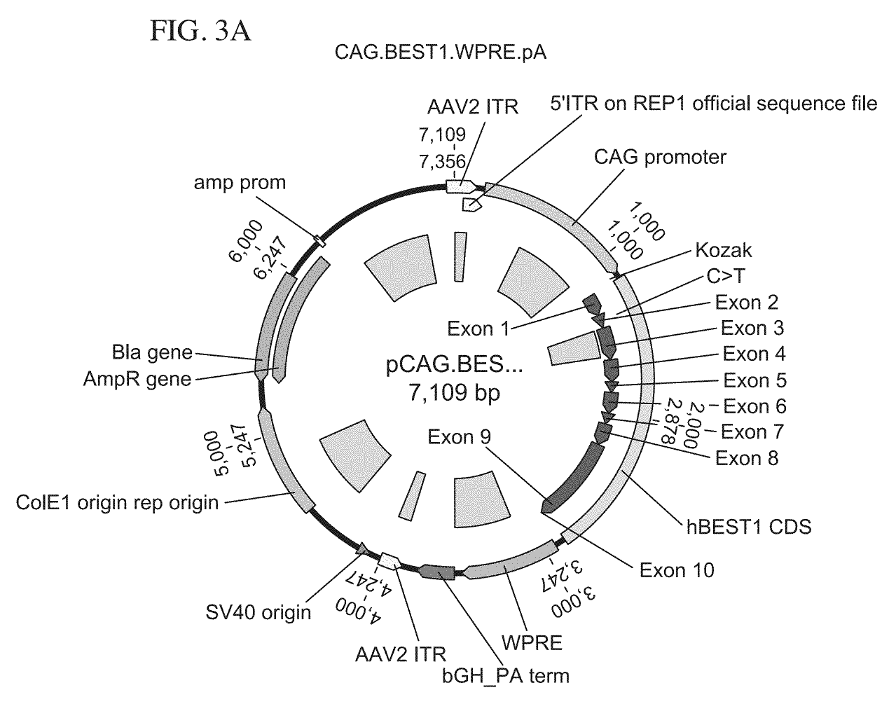

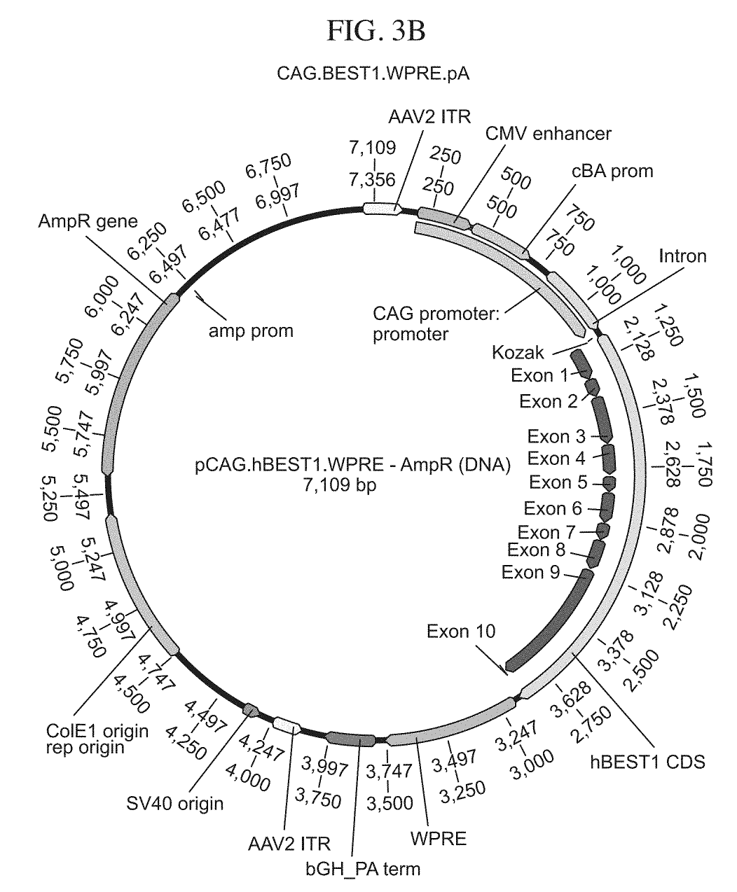

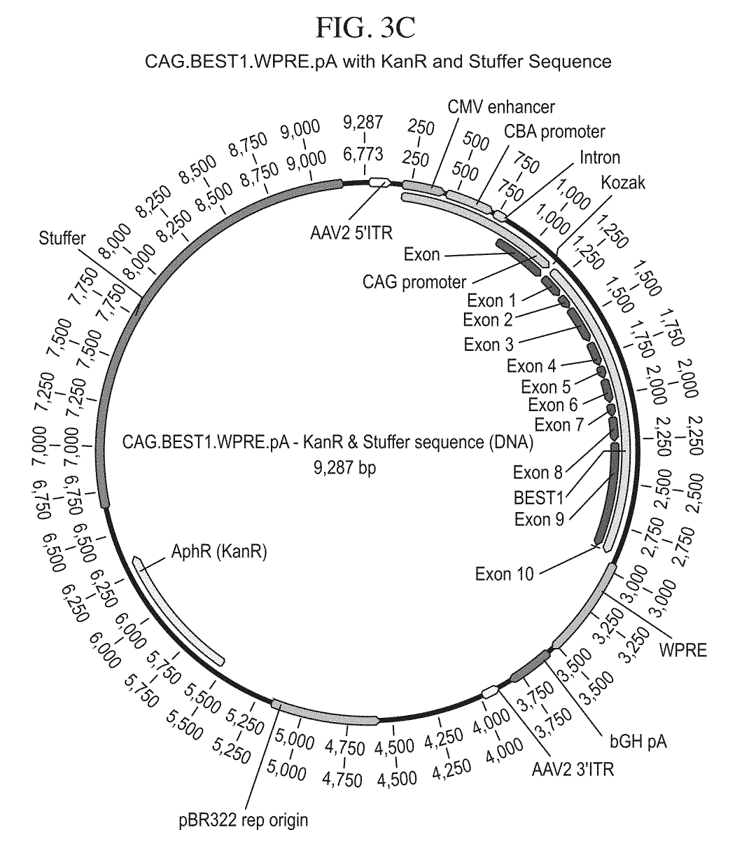

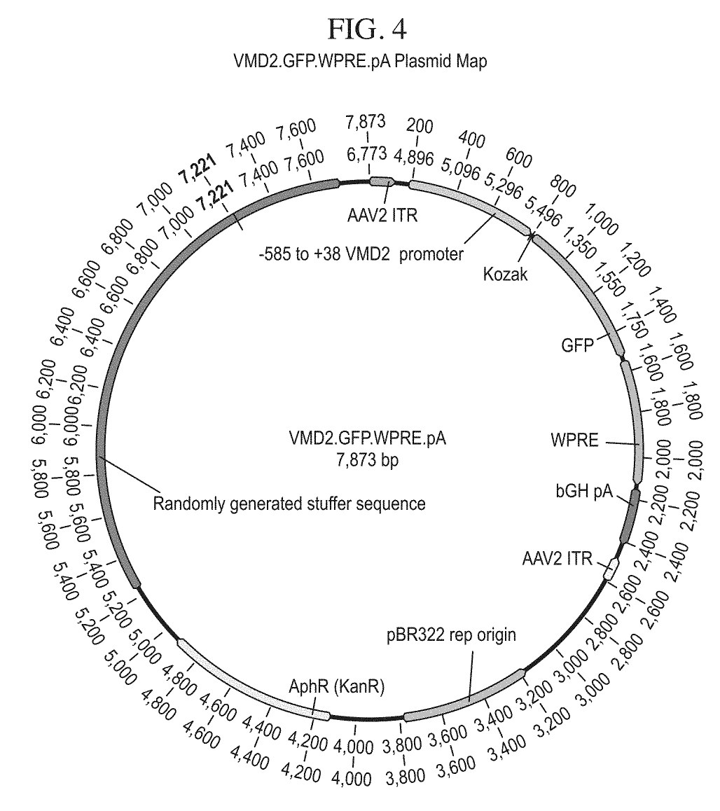

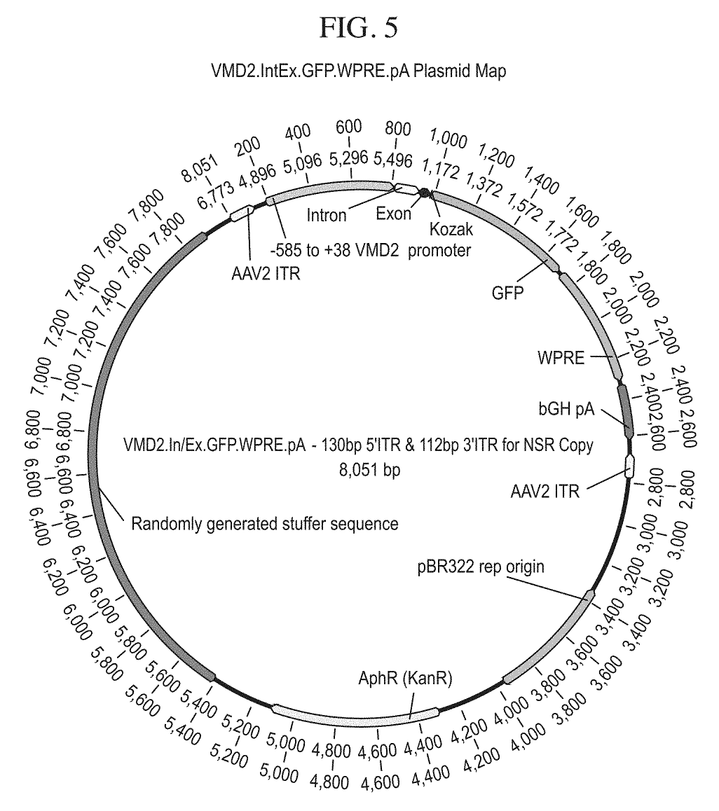

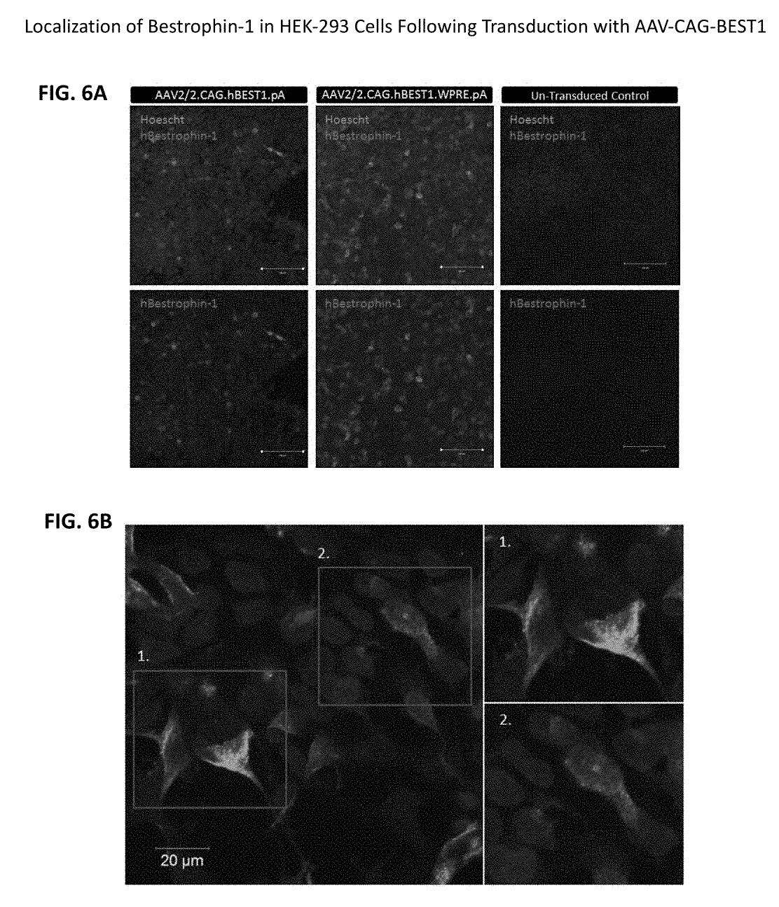

HEK293 cells were transduced with an AAV2/2 vector containing the CAG promoter driving Best1 expression with a WPRE (AAV2/2 CAG.BEST1.WPRE.pA, FIG. 3) and without a WPRE (AAV2/2 CAG.BEST1.pA), and the expression and localization of Bestrophin-1 protein was examined. In FIG. 6, transduced HEK293 cells were stained with Hoechst and an anti-human Bestrophin-1 (hBEST1 or huBEST1) antibody. Bestrophin-1 protein was found throughout the cytosol when compared to untransduced control cells.

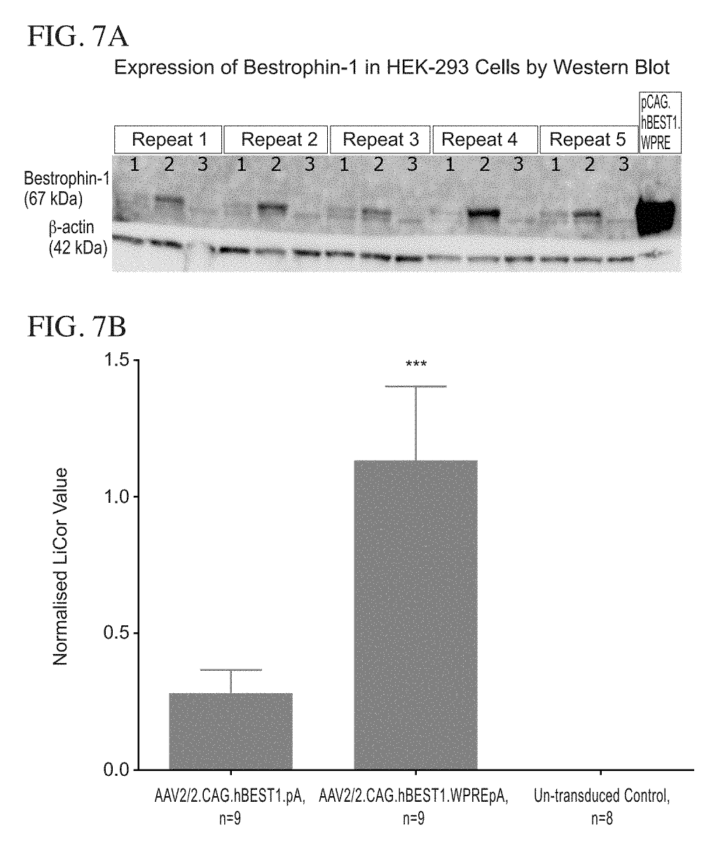

Bestrophin-1 expression in HEK293 cells was quantified from Western Blot (FIG. 7). In FIG. 7A, sample 1 was the AAV2/2 CAG.hBEST1.pA vector; sample 2 was the AAV2/2 CAG.hBEST1.WPRE.pA vector and sample 3 was a negative control. Plasmid-transfected HEK293 cells were used as a positive control. In FIG. 7B, quantification showed that AAV2/2 CAG.BEST1.WPRE.pA (n=9) showed an approximately 4-fold increase in Bestrophin-1 expression over AAV2/2 CAG.BEST1.pA (n=9) (p<0.01 by One-way ANOVA with Tukey's Multiple Comparisons Test) and a statistically significant increase over un-transduced control cells (n=8) (p<0.001) was seen. Although Bestrophin-1 expression was seen in the AAV2/2 CAG.BEST1.pA cells, this was not statistically significant over un-transduced cells. Error bars=±SEM, and *** indicates p<0.001 when compared to un-transduced control.

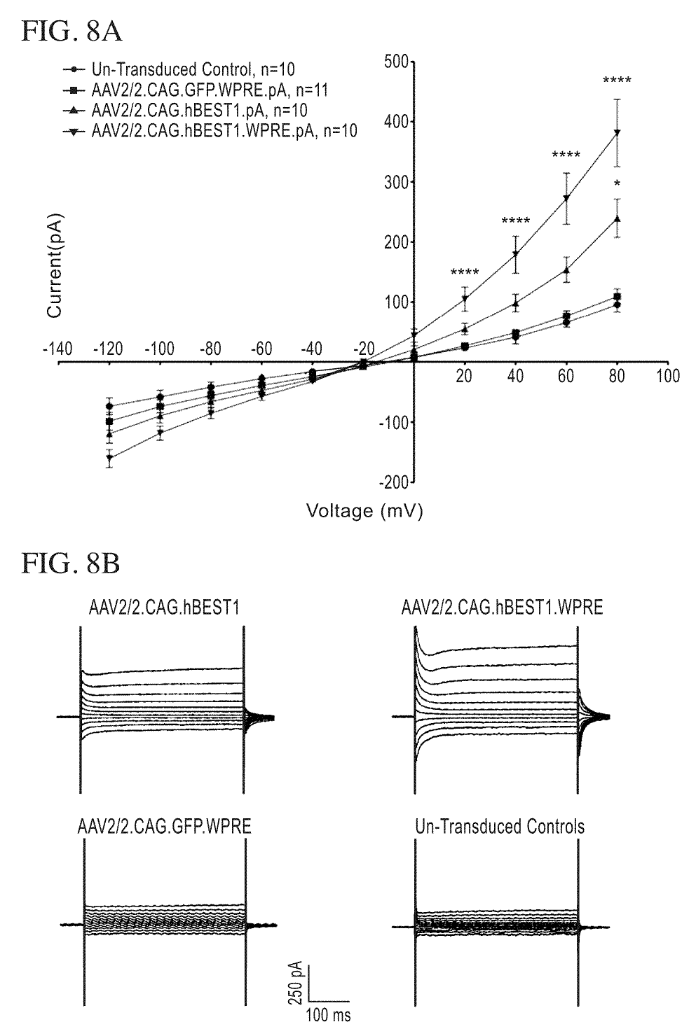

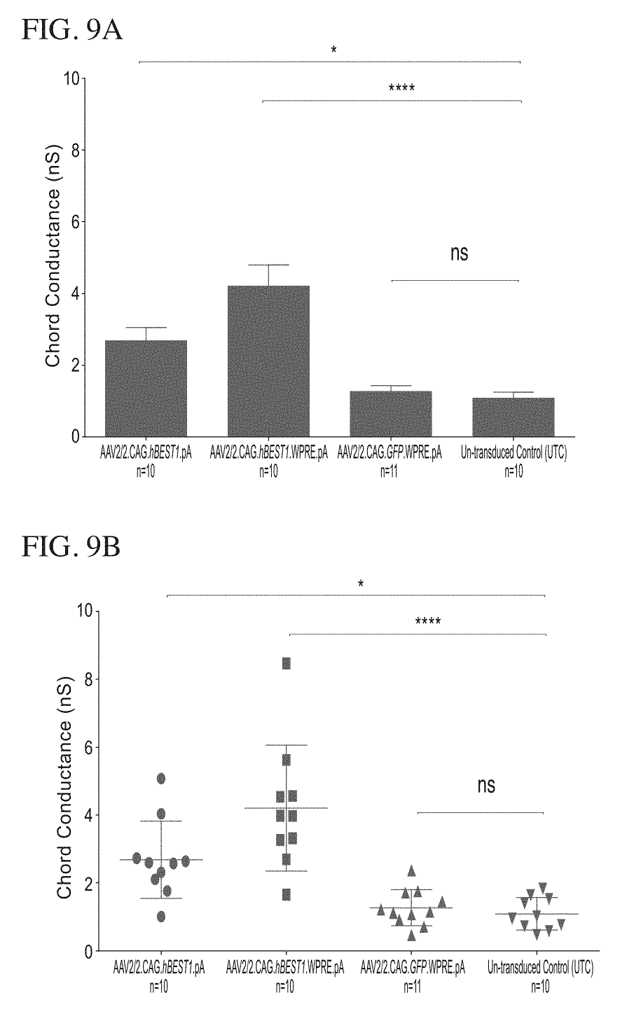

HEK293 cells expressing Bestrophin-1 were additionally assayed with whole-cell patch clamp recording. FIG. 8A shows the Current (I)/Voltage (V) plots of HEK293 cells transduced with AAV2/2 CAG.BEST1.pA, AAV2/2 CAG.BEST1.WPRE.pA and AAV2/2 CAG.GFP.WPRE.pA vectors as well as an untransduced control. FIG. 8B shows the current waveforms, and chord conductance is shown in FIG. 9.

### Example 2: Bestrophin-1 Protein in Cultured ARPE19 Cells Using the VMD2 Promoter

Appropriately differentiated ARPE19 are known to have gene expression profiles similar to those of native retinal pigment epithelium (RPE) cells, and can be used as an alternative to native RPE cells to test gene expression. Differentiated ARPE19 cells were used to test the ability of the VMD2 and CAG promoters to drive BEST1 expression in RPE cells, and to test the effect of the intron-exon (IntEx) sequence on expression from the VMD2 promoter.

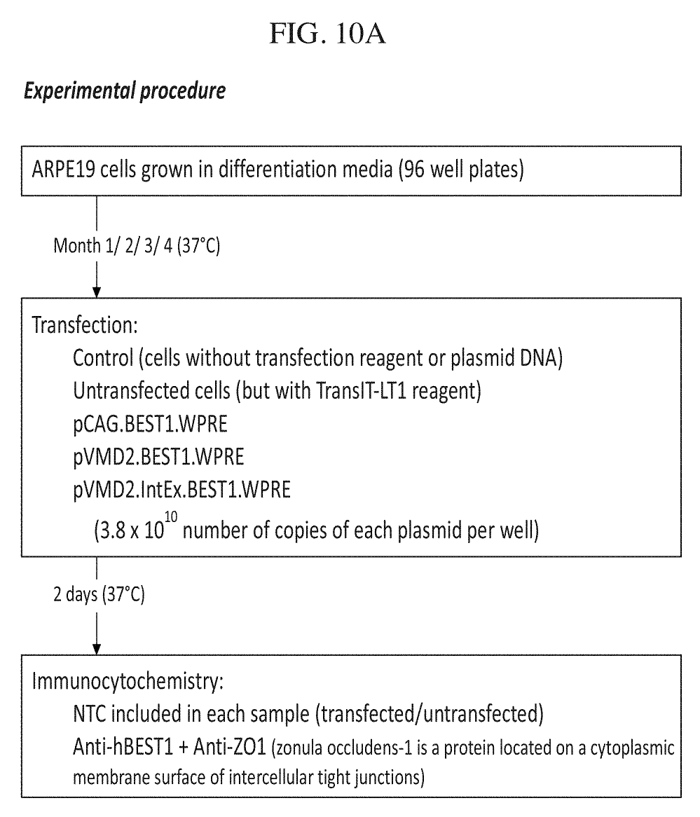

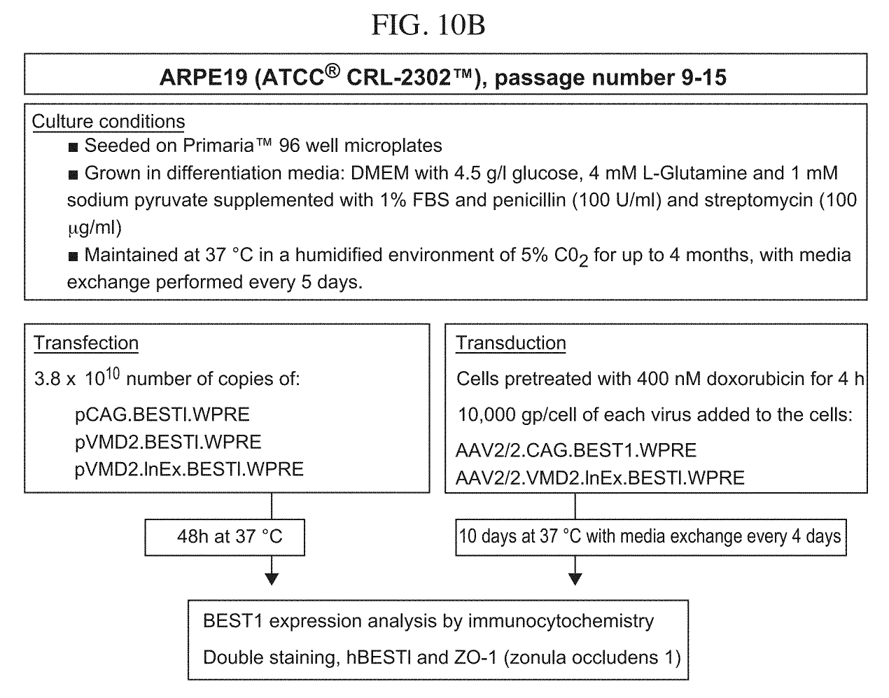

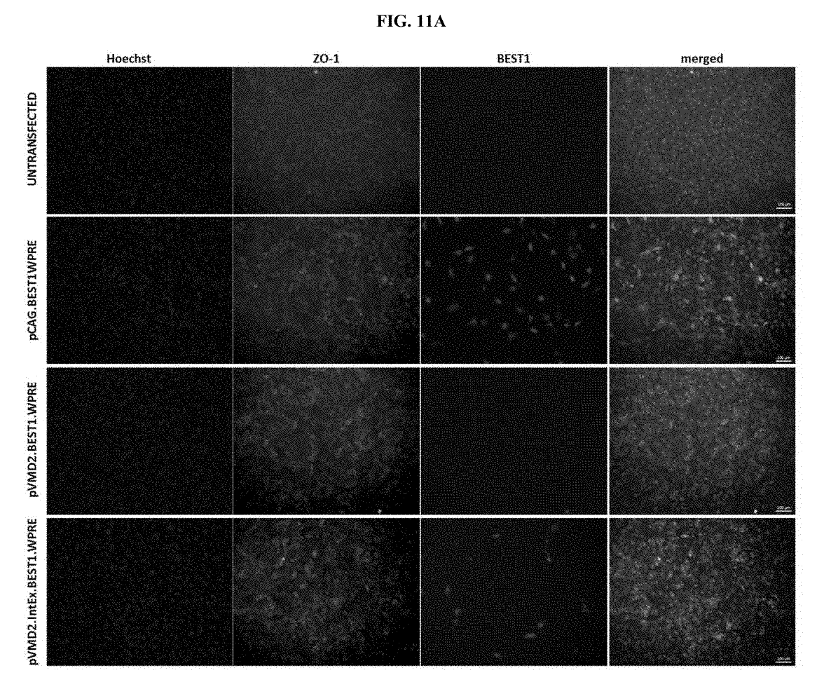

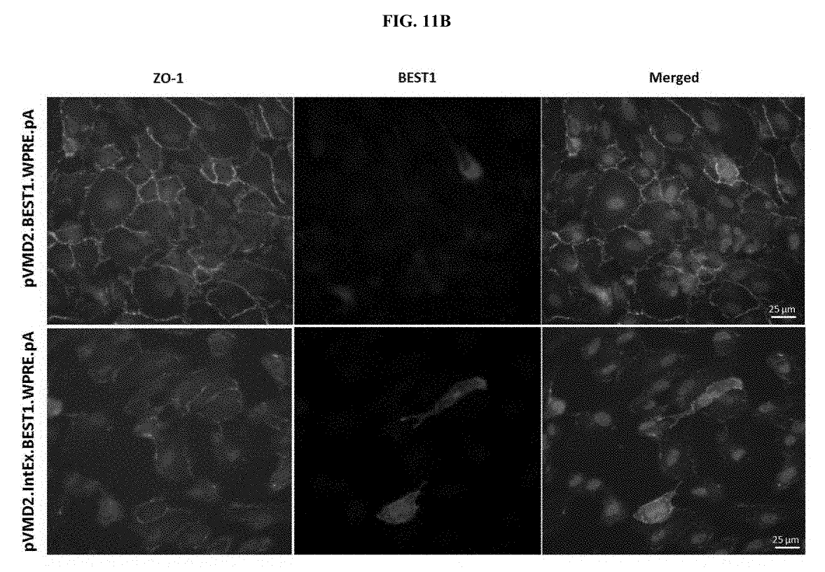

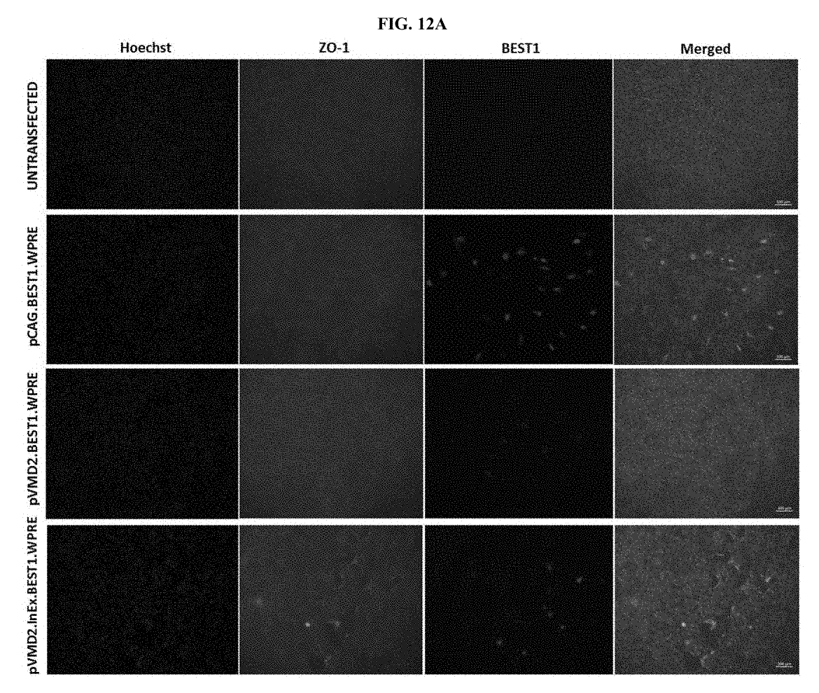

ARPE19 cells were transfected and assayed for BEST1 expression using the protocol outlined in FIG. 10B. ARPE19 cells were grown in differentiation medium (DMEM with 4.5 g/l glucose, L-glutamine, and 1 mM sodium pyruvate supplemented with 1% fetal bovine serum (FBS) for 1-4 months at 37° C. and 5% CO₂ in 96 well plates. Differentiated ARPE19 cells were then transfected with either pCAG.BEST1.WPRE (CAG promoter), pVMD.BEST1.WPRE (VMD2 promoter), or pVMD2.IntEx.BEST1.WPRE (VMD2 promoter and an intron-exon construct) at 3.8×10¹⁰ number of copies of each plasmid per well. Cells treated with TransIT-LT1 reagent alone and cells without transfection reagent or plasmid served as negative controls. Cells were then cultured for 2 days at 37° C., before being fixed and stained with Anti-hBest1 and Anti-ZO1 (also called ZO-1, or zona occludens-1, or tight junction protein 1, a protein located on the cytoplasmic membrane surface of intercellular tight junctions). FIGS. 11-13 show BEST1 expression in differentiated ARPE19 cells transfected with the three vectors encoding BEST1 and an untransfected control.

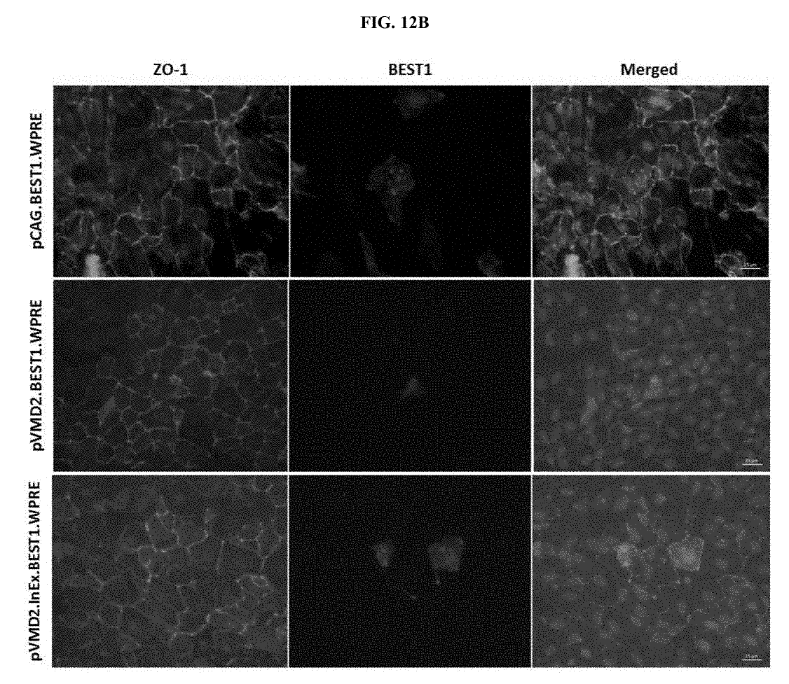

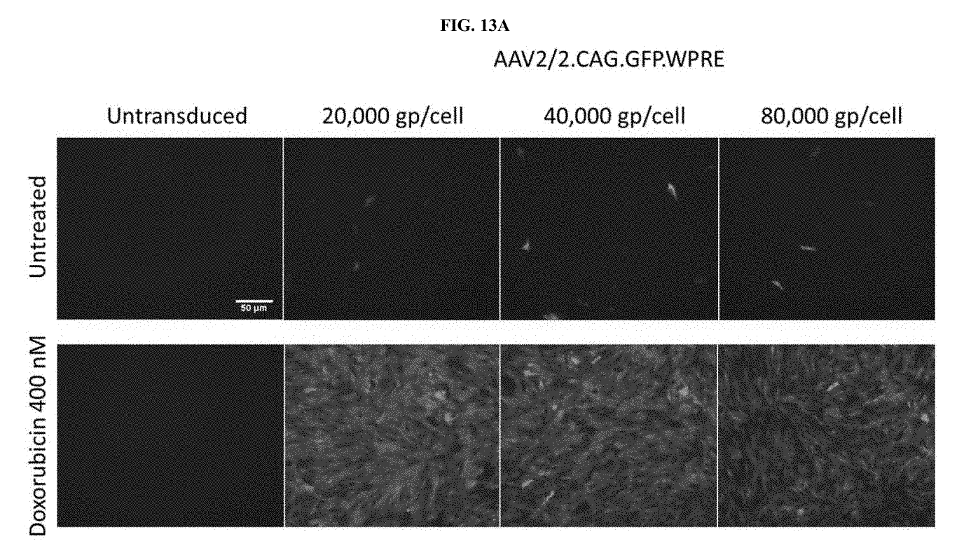

In ARPE19 cells that were differentiated for one month before transfection, the untransfected cells showed no expression. In contrast, both the pCAG.BEST1.WPRE and pVMD2.IntEx.BEST1.WPRE were able to drive the expression of BEST1 protein in differentiated ARPE19 cells (see FIG. 11A, contrast the first, second and fourth rows). pVMD2.BEST1.WPRE (no exon-intron) was also able to drive the expression of BEST1 in 1 month differentiated ARPE19 cells (FIG. 11B), although this construct seemed to express BEST1 at lower levels than the construct with the intron-exon sequence (pVMD2.IntEx.BEST1.WPRE). In ARPE19 cells that were differentiated for three months, similar results were obtained: pCAG.BEST1.WPRE, pVMD2.IntEx.BEST1.WPRE and pVMD2.BEST1.WPRE were all able to drive expression of BEST1 protein, although the expression with the CAG promoter was higher than with the VMD2 promoter and, with the VMD2 promoter, the intron-exon sequence improves the expression (contrast the first row of FIG. 12A, the untransfected control, with FIG. 12B).

ARPE19 cells were transduced and assayed for BEST1 expression using the protocol outlined in FIG. 10. Differentiated ARPE19 cells were pre-treated with 400 nM doxorubicin before transduction. This drug has been proved to improve AAV2 transduction efficiency in several in vitro models. Four hours after the treatment, cells were transduced with the different viral constructs at different multiplicities of infection (MOIs).

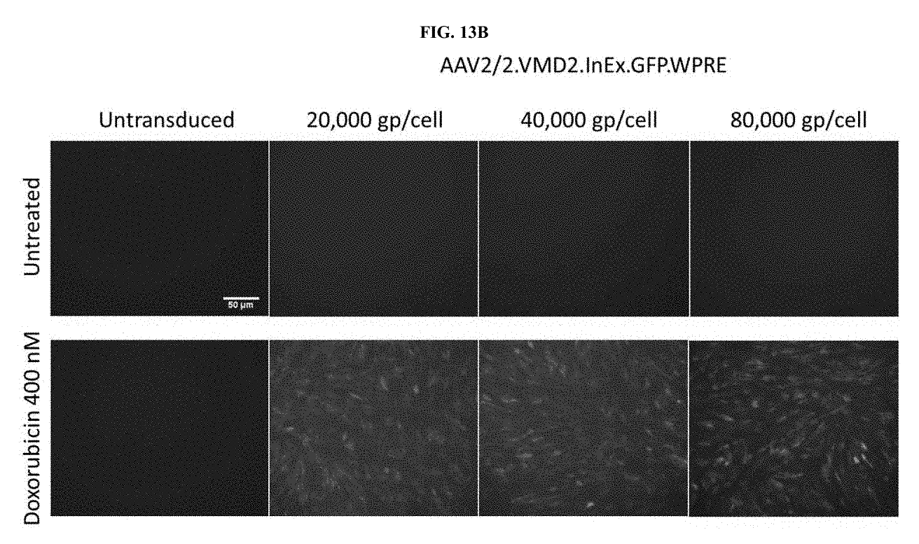

ARPE19 cells differentiated for 4 months, pre-treated with 400 nM doxorubicin and transduced with AAV2/2.CAG.GFP.WPRE and AAV2/2.VMD2.InEx.GFP.WPRE at 2, 4 and 8×10⁴ gp/cell showed higher GFP fluorescence compared to transduced cells without pre-treatment with doxorubicin 10 days after transduction (contrast top and bottom row of each panel of FIG. 13A) AAV2/2.CAG.GFP.WPRE and B) AAV2/2.VMD2.InEx.GFP.WPRE). GFP fluorescence was not detected in untransduced cells, used as negative controls (first column of FIGS. 13A and B).

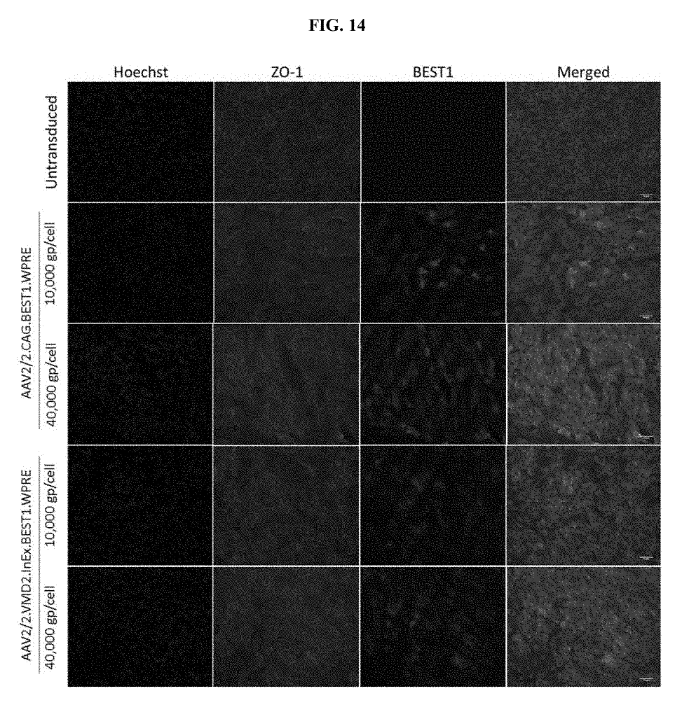

In ARPE19 cells differentiated for 4 months, pre-treated with 400 nM doxorubicin and transduced with AAV2/2.CAG.BEST1.WPRE and AAV2/2.VMD2.InEx.BEST1.WPRE at 1 and 4×10⁴ gp/cell, BEST1 expression could be detected by immunostaining with anti-hBEST1 (red, third column, second to fifth row of FIG. 14) compared to the untransduced control (first row, FIG. 14) 10 days after transduction.

### Example 3: 4/8 Week In Vivo Pilot Study in Mice

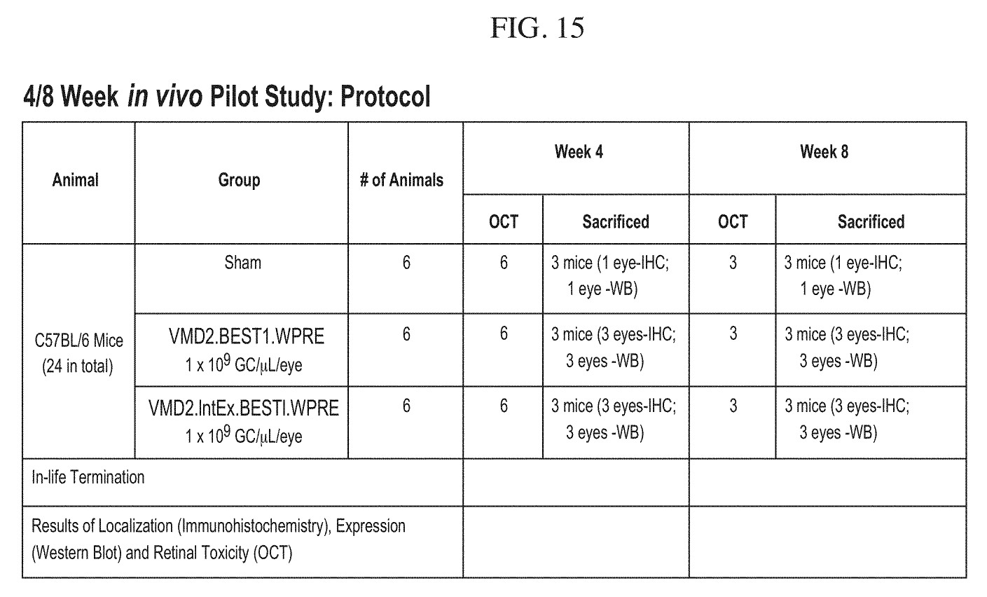

The ability of the VMD2.BEST1.WPRE and VMD2.IntEx.BEST1.WPRE constructs to drive the expression BEST1 was assayed in vivo. The protocol of the 4/8 week in vivo pilot study is shown in FIG. 15. C57BL/6 mice (6 per group) were injected bilaterally with either a sham injection, AAV2/2 VMD2.BEST1.WPRE or AAV2/2 VMD2.IntEx.BEST1.WPRE AAV viral particles. 1 μL of AAV solution was injected subretinally with a 34 gauge Nanofil needle (WPI #NF34BL-2) at 1×10⁹ GC/μL/eye. Eyes were imaged using optical coherence tomography at 4 and 8 weeks to assess for retinal thinning (toxicity), and 3 animals were sacrificed at each time point to assay BEST1 protein expression by immunohistochemistry and Western Blot.

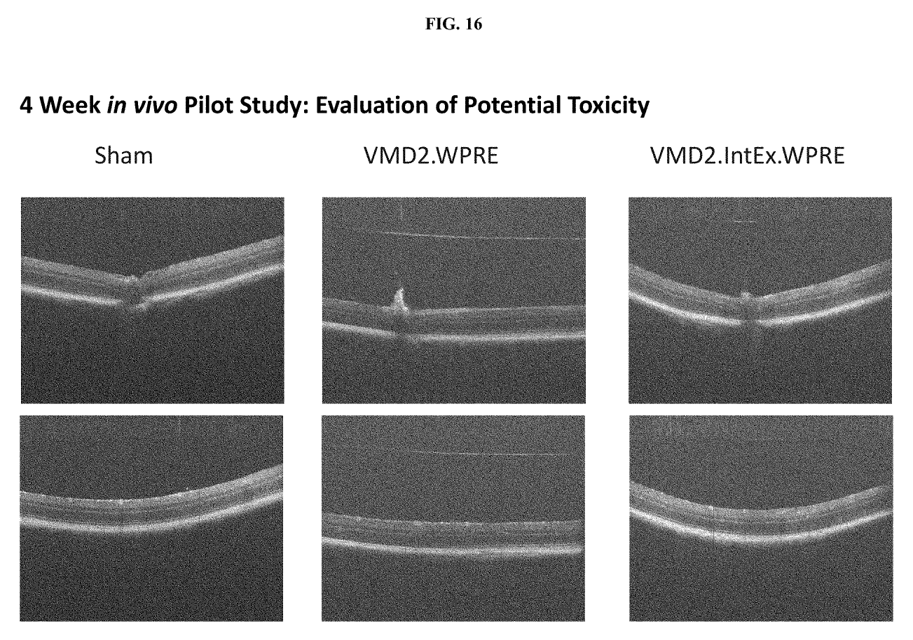

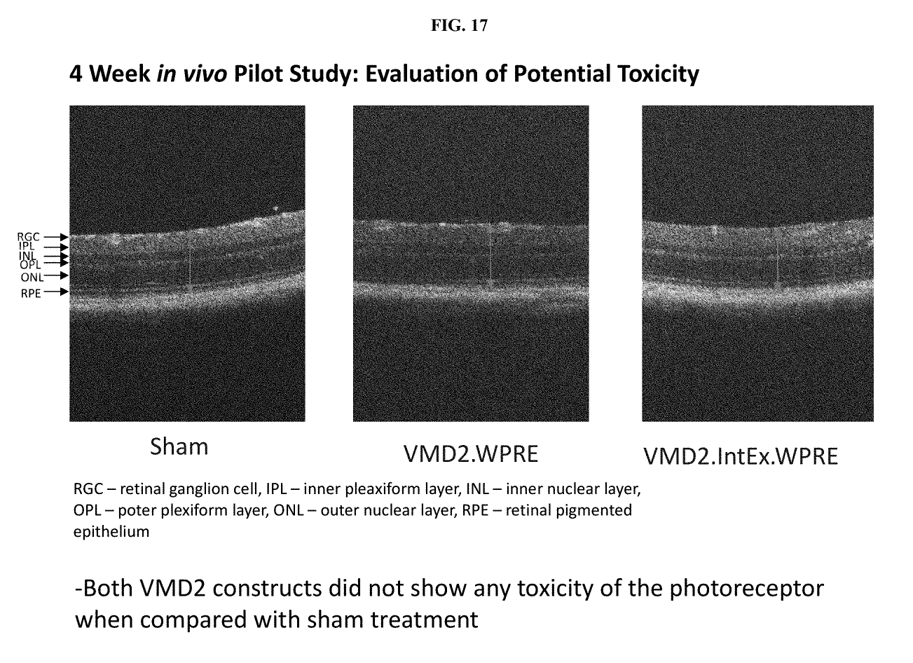

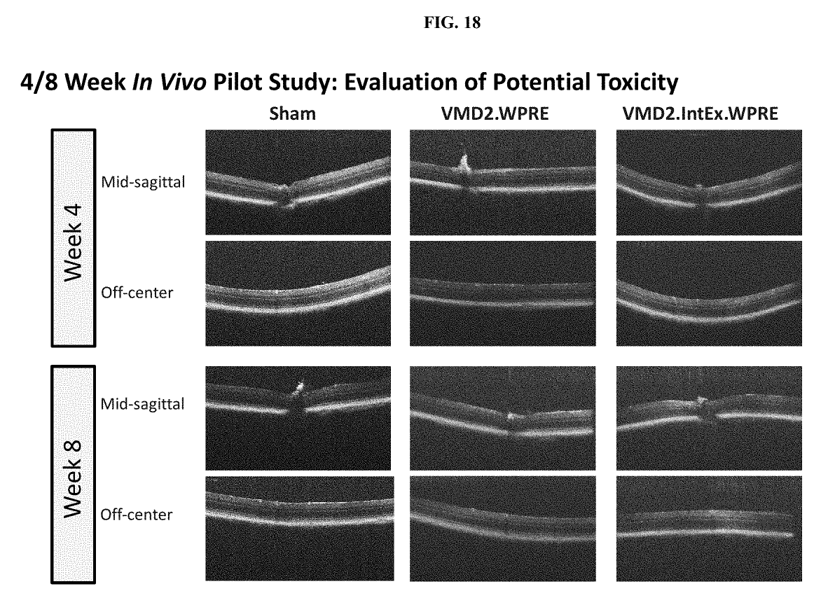

OCT imaging at 4 and 8 weeks showed that neither VMD2 construct showed photoreceptor toxicity when compared to the sham treatment (FIGS. 16-18).

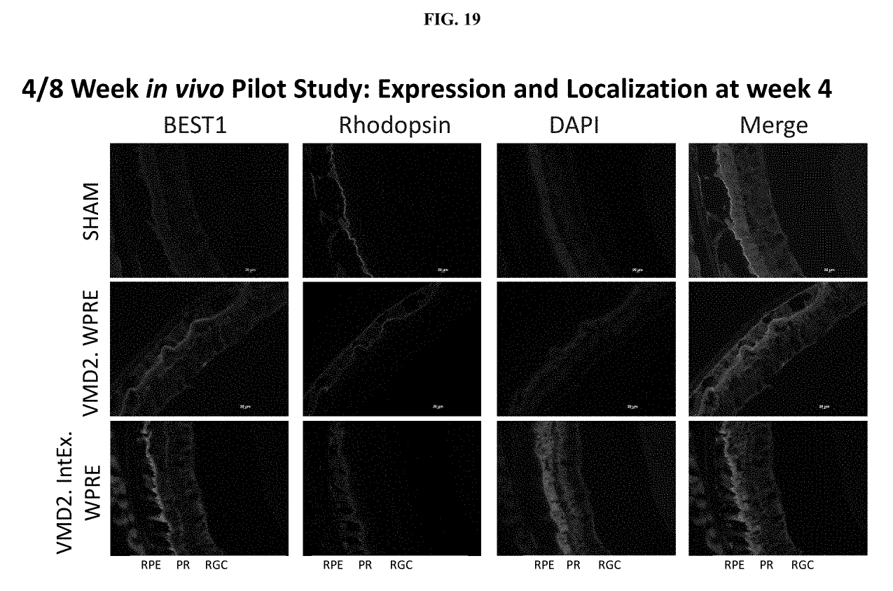

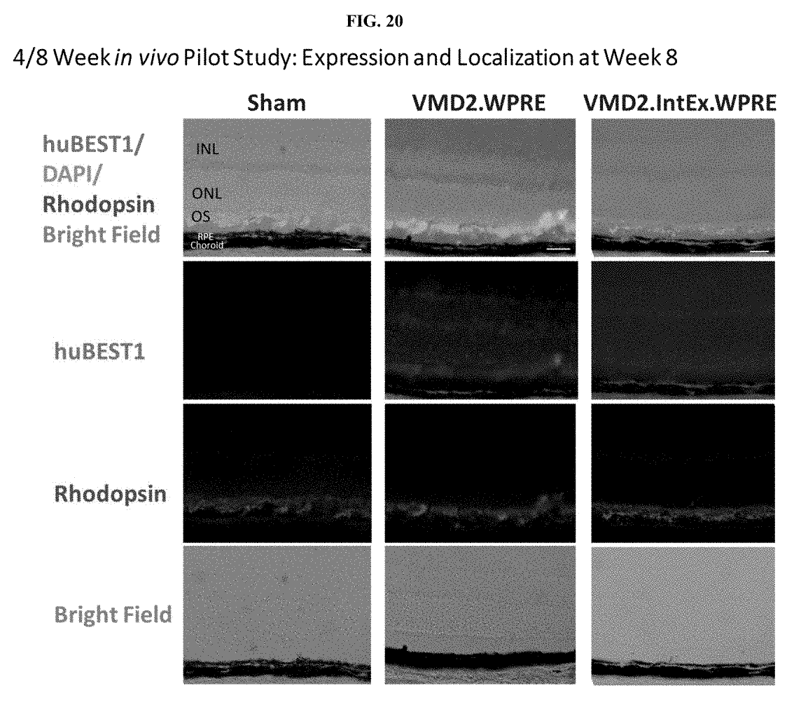

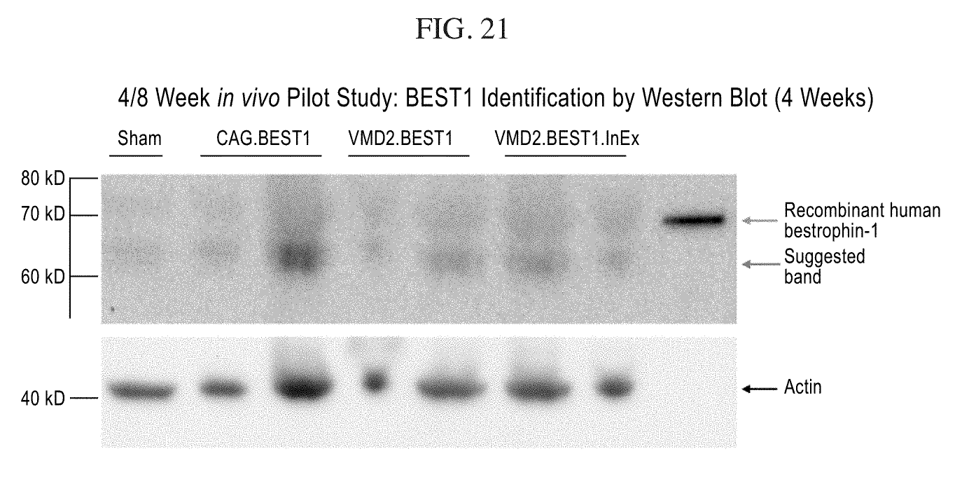

Three animals were sacrificed at both the 4- and 8-week time points, and BEST1 protein expression was further characterized by western blot (FIG. 21) and immunohistochemistry (FIG. 19). FIG. 19 shows immunohistochemistry results for eyes four weeks post injection, while FIG. 20 shows immunohistochemistry results for eyes eight weeks post injection. Eyes were stained with anti-BEST1 (green) and anti-Rhodopsin (red), which marks photoreceptor cells, and DAPI (FIGS. 19 and 20). BEST1 protein expression was observed from VMD2.BEST1.WPRE.pA and VMD2.IntEx.BEST1.WPRE.pA. VMD2 promoter driven BEST1 expression localized to the conjunction of the RPE layer and photoreceptor outer layer. Western Blot on dissected RPE/choroid complex tissue from four week injected eyes shows protein expression (FIG. 21).

### Example 4: 4/13 Week In Vivo Proof of Concept Study in Mice

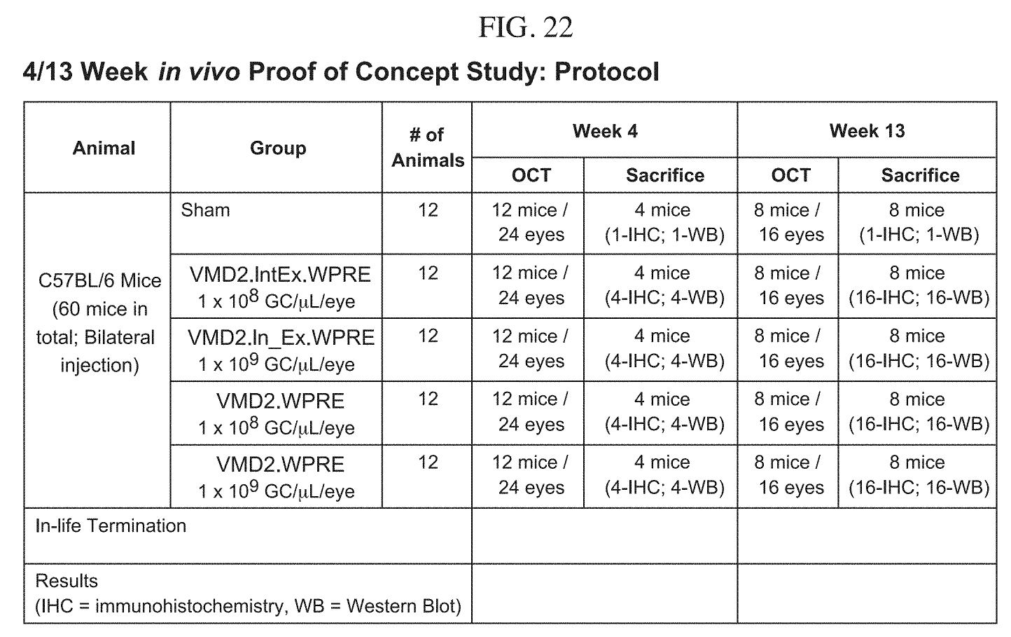

An additional 4 and 13 week in vivo proof of concept (PoC) study was carried out in mice to confirm the results of the pilot study, assay the effect of AAV viral particle dosage, and look at the effects at later time points post AAV injection. An outline of the protocol for the 4/13 week Proof of Concept study is set forth in FIG. 22. C57BL/6 mice (12 per cohort) were bilaterally injected with VMD2.IntEx.BEST1.WPRE or VMD2.BEST1.WPRE.pA AAV particles at either 1×10⁸ GC/μL/eye or 1×10⁹ GC/μL/eye, or with a sham injection. 1 μL of AAV solution was injected subretinally with a 34 gauge Nanofil needle (WPI #NF34BL-2). Eyes were imaged with OCT at 4 and 13 weeks post injection. Four mice were sacrificed four weeks post injection, and the remaining eight at 13 weeks post injection, and BEST1 expression was characterized by immunohistochemistry and Western blot.

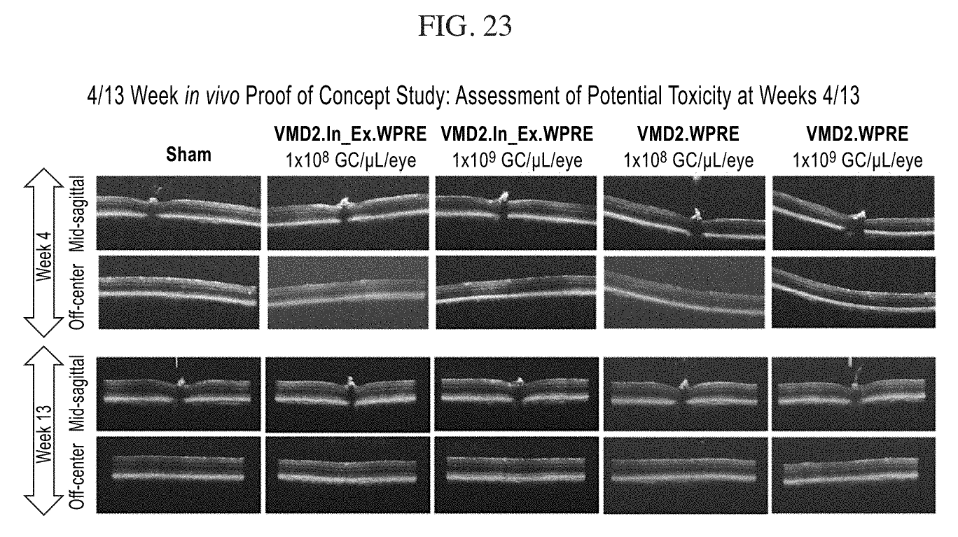

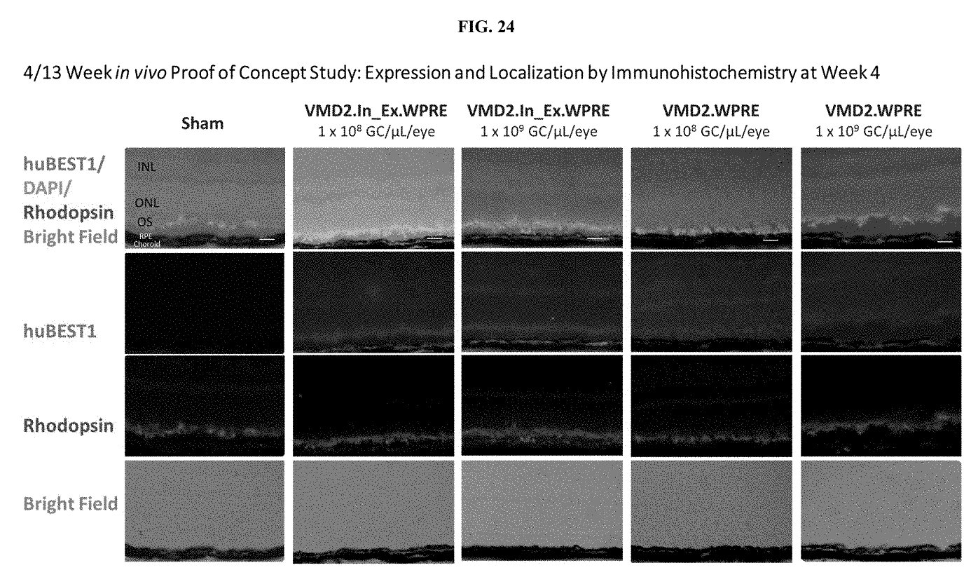

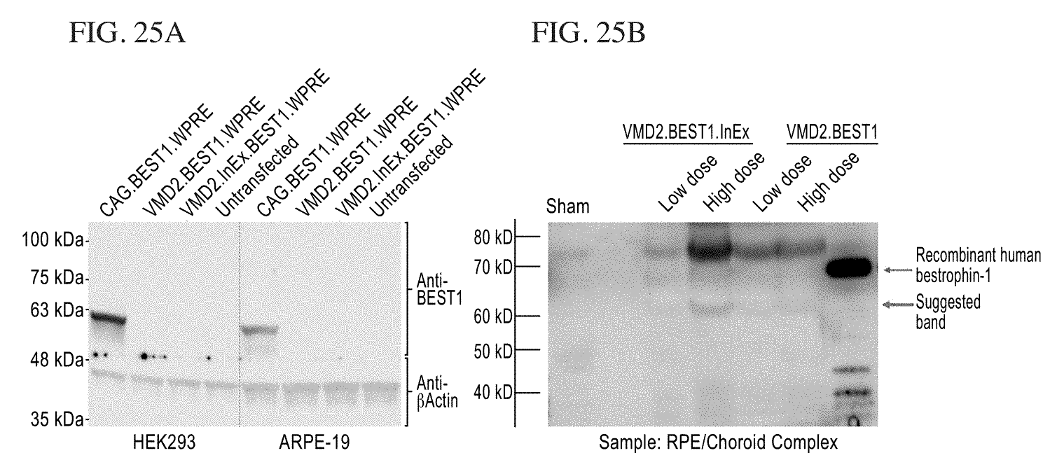

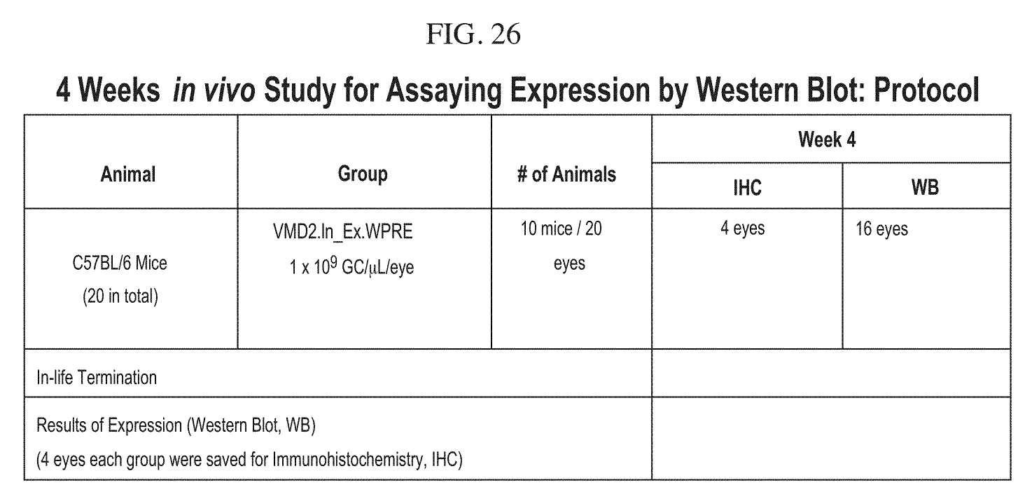

OCT imaging at 4 weeks and 13 weeks showed that neither VMD2 construct (with or without the intron-exon sequence) at either the high dose (1×10⁹ GC/eye) or the low dose (1×10⁸ GC/eye) showed toxicity as evidenced by retinal thinning when compared to the sham control (FIG. 23). Staining with anti-BEST1 (huBEST1 in FIG. 24) and anti-Rhodopsin showed that VMD2 driven BEST1 localized to the RPE layer, with a trend of more BEST1 expression in VMD2.IntEx.BEST1.WPRE injected eyes. Western Blot on pooled dissected RPE/choroid complex tissue from four week injected eyes (4) shows protein expression (FIG. 25B).

### Example 5: Good Laboratory Practice (GLP) Toxicity Assessment Study in Mice

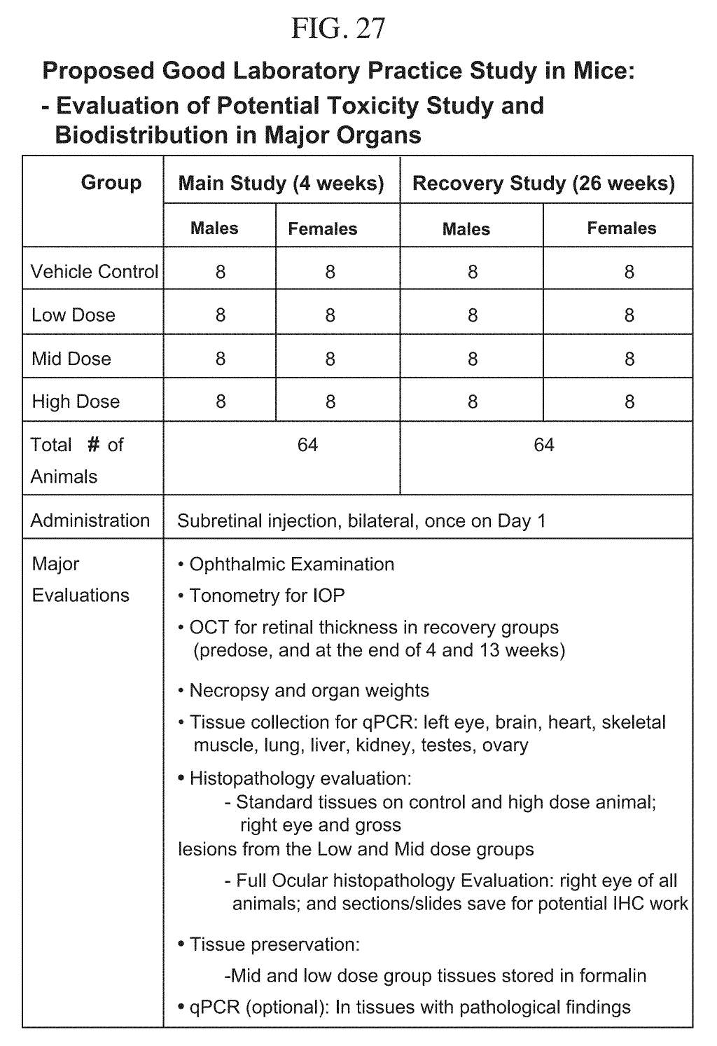

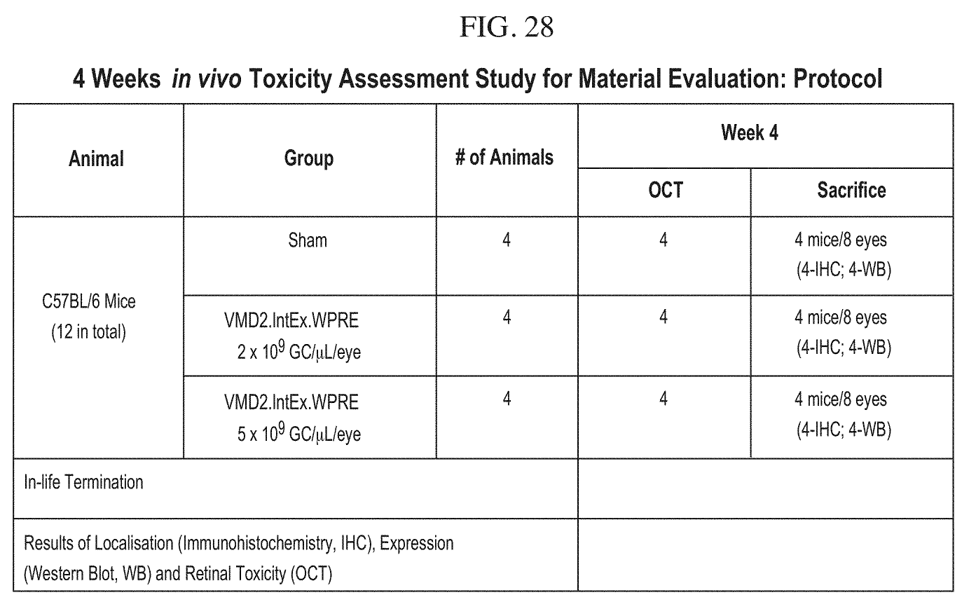

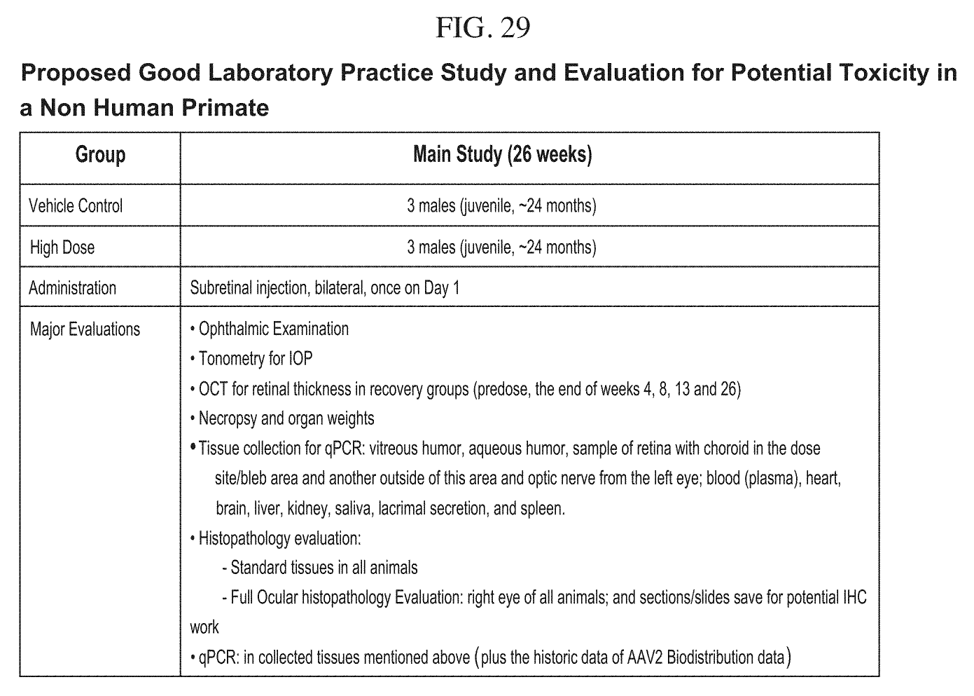

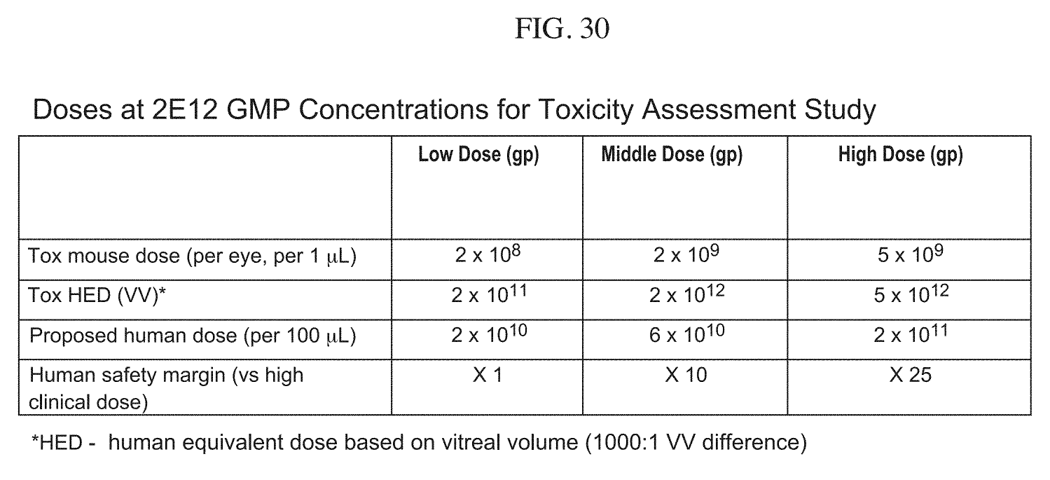

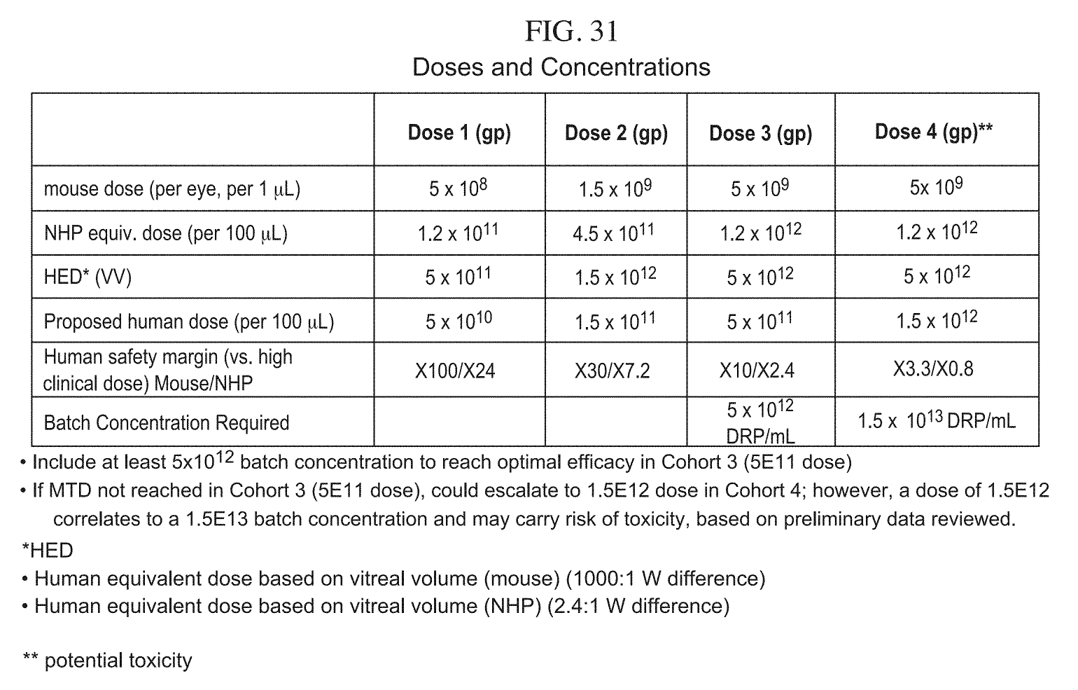

The safety and expression of BEST1 AAV over longer periods of time is verified in mice with a Good Laboratory Practice (GLP) toxicity study in mice. An outline of the study is set forth in FIG. 27. Cohorts of 8 male and 8 female mice are injected subretinally and bilaterally with a low (5.0×10⁸ GC/eye), medium (1.5×10⁹ GC/eye) or high (5.0×10⁹ GC/eye) dose of VMD2.IntEx.BEST1.WPRE AAV particles. Using allometric volume scaling, the high mouse dose is equivalent to a dose of 100 μL at 5×10¹² GC/mL/eye in humans. Mice are evaluated and sacrificed at 4 weeks and 26 weeks. Eyes are assessed with an ophthalmic examination, tonometry to measure intraocular pressure (IOP), OCT for retinal thickness (predose, and the end of 4 and 13 weeks). Post sacrifice, necropsies assess organ weights and tissues such as the left eye, brain, heart, skeletal muscle, lung, liver, kidney, testes and ovary are collected for qPCR. Histopathological evaluations are carried out, and tissues are reserved, e.g. by storage in formalin, for additional immunohistochemistry. Alternatively, or in addition, groups of 4 mice are injected with dosages of 2×10⁹ GC/eye and 5×10⁹ GC/eye of VMD2.IntEx.BEST1.WPRE AAV particles and evaluated at 4 weeks to optimize protocols for the larger toxicity study (see FIG. 28 for an outline).

### INCORPORATION BY REFERENCE

Every document cited herein, including any cross referenced or related patent or application is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

### Other Embodiments

While particular embodiments of the disclosure have been illustrated and described, various other changes and modifications can be made without departing from the spirit and scope of the disclosure. The scope of the appended claims includes all such changes and modifications that are within the scope of this disclosure.

1. A composition comprising:
a nucleic acid sequence comprising:
(a) a sequence encoding a vitelliform macular dystrophy-2 (VMD2) promoter, and
(b) a sequence encoding a Bestrophin-1 (BEST1) protein.

2. The composition of claim 1, wherein the sequence encoding the VMD2 promoter encodes a human VMD2 promoter.

3. The composition of claim 1, wherein the sequence encoding the BEST1 protein encodes a human BEST1 protein.

4.-5. (canceled)

6. The composition of claim 1, wherein the nucleic acid sequence further comprises one or more of the following:
(c) a sequence encoding a posttranscriptional regulatory element (PRE), optionally wherein the sequence encoding the PRE comprises a sequence isolated or derived from a woodchuck hepatitis virus (WPRE);
(d) a sequence encoding a polyadenylation (polyA) signal;
(e) a sequence encoding a 5′ untranslated region;
(f) a sequence encoding an intron; or
(g) a sequence encoding an exon, wherein the sequence encoding the intron and the sequence encoding the exon are operably linked.

7. (canceled)

8. The composition of claim 6, wherein the nucleic acid sequence further comprises:
(c) a sequence encoding a posttranscriptional regulatory element (PRE) isolated or derived from a woodchuck hepatitis virus (WPRE);
(d) a sequence encoding a polyadenylation (polyA) signal;
(e) a sequence encoding a 5′ untranslated region;
(f) a sequence encoding an intron; and
(g) a sequence encoding an exon, wherein the sequence encoding the intron and the sequence encoding the exon are operably linked.

9.-10. (canceled)

11. The composition of claim 6,
wherein the sequence encoding the intron is located between the sequence encoding the VMD2 promoter and the sequence encoding the exon,
wherein the sequence encoding the exon is located between the sequence encoding the intron and the sequence encoding the 5′ UTR, and
wherein the sequence encoding the intron is spliced by a mammalian cell.

12. The composition of claim 6, wherein the sequence encoding the 5′ UTR comprises a sequence encoding a Kozak sequence or a portion thereof.

13. The composition of claim 12, wherein the sequence encoding a Kozak sequence has at least 50% identity to the nucleic acid sequence of GCCRCCATGG where R represents an A or G, and optionally wherein the sequence encoding a Kozak sequence comprises or consists of the nucleic acid sequence of GGCACCATGA.

14. (canceled)

15. The composition of claim 2, wherein the sequence encoding the human VMD2 promoter comprises or consists of

| (SEQ ID NO: 1) | |
|---|---|
| 1 | AATTCTGTCA TTTTACTAGG GTGATGAAAT TCCCAAGCAA CACCATCCTT TTCAGATAAG |
| 61 | GGCACTGAGG CTGAGAGAGG AGCTGAAACC TACCCGGGGT CACCACACAC AGGTGGCAAG |
| 121 | GCTGGGACCA GAAACCAGGA CTGTTGACTG CAGCCCGGTA TTCATTCTTT CCATAGCCCA |
| 181 | CAGGGCTGTC AAAGACCCCA GGGCCTAGTC AGAGGCTCCT CCTTCCTGGA GAGTTCCTGG |
| 241 | CACAGAAGTT GAAGCTCAGC ACAGCCCCCT AACCCCCAAC TCTCTCTGCA AGGCCTCAGG |
| 301 | GGTCAGAACA CTGGTGGAGC AGATCCTTTA GCCTCTGGAT TTTAGGGCCA TGGTAGAGGG |
| 361 | GGTGTTGCCC TAAATTCCAG CCCTGGTCTC AGCCCAACAC CCTCCAAGAA GAAATTAGAG |
| 421 | GGGCCATGGC CAGGCTGTGC TAGCCGTTGC TTCTGAGCAG ATTACAAGAA GGGACTAAGA |
| 481 | CAAGGACTCC TTTGTGGAGG TCCTGGCTTA GGGAGTCAAG TGACGGCGGC TCAGCACTCA |
| 541 | CGTGGGCAGT GCCAGCCTCT AAGAGTGGGC AGGGGCACTG GCCACAGAGT CCCAGGGAGT |
| 601 | CCCACCAGCC TAGTCGCCAG ACC. |

16. The composition of claim 3, wherein the sequence encoding the human BEST1 protein comprises or consists of

| (SEQ ID NO: 3) | |
|---|---|
| 1 | ATGACCATCA CTTACACAAG CCAAGTGGCT AATGCCCGCT TAGGCTCCTT CTCCCGCCTG |
| 61 | CTGCTGTGCT GGCGGGGCAG CATCTACAAG CTGCTATATG GCGAGTTCTT AATCTTCCTG |
| 121 | CTCTGCTACT ACATCATCCG CTTTATTTAT AGGCTGGCCC TCACGGAAGA ACAACAGCTG |
| 181 | ATGTTTGAGA AACTGACTCT GTATTGCGAC AGCTACATCC AGCTCATCCC CATTTCCTTC |
| 241 | GTGCTGGGCT TCTACGTGAC GCTGGTCGTG ACCCGCTGGT GGAACCAGTA CGAGAACCTG |
| 301 | CCGTGGCCCG ACCGCCTCAT GAGCCTGGTG TCGGGCTTCG TCGAAGGCAA GGACGAGCAA |
| 361 | GGCCGGCTGC TGCGGCGCAC GCTCATCCGC TACGCCAACC TGGGCAACGT GCTCATCCTG |
| 421 | CGCAGCGTCA GCACCGCAGT CTACAAGCGC TTCCCCAGCG CCCAGCACCT GGTGCAAGCA |
| 481 | GGCTTTATGA CTCCGGCAGA ACACAAGCAG TTGGAGAAAC TGAGCCTACC ACACAACATG |
| 541 | TTCTGGGTGC CCTGGGTGTG GTTTGCCAAC CTGTCAATGA AGGCGTGGCT TGGAGGTCGA |
| 601 | ATCCGGGACC CTATCCTGCT CCAGAGCCTG CTGAACGAGA TGAACACCTT GCGTACTCAG |
| 661 | TGTGGACACC TGTATGCCTA CGACTGGATT AGTATCCCAC TGGTGTATAC ACAGGTGGTG |
| 721 | ACTGTGGCGG TGTACAGCTT CTTCCTGACT TGTCTAGTTG GGCGGCAGTT TCTGAACCCA |
| 781 | GCCAAGGCCT ACCCTGGCCA TGAGCTGGAC CTCGTTGTGC CCGTCTTCAC GTTCCTGCAG |
| 841 | TTCTTCTTCT ATGTTGGCTG GCTGAAGGTG GCAGAGCAGC TCATCAACCC CTTTGGAGAG |
| 901 | GATGATGATG ATTTTGAGAC CAACTGGATT GTCGACAGGA ATTTGCAGGT GTCCCTGTTG |
| 961 | GCTGTGGATG AGATGCACCA GGACCTGCCT CGGATGGAGC CGGACATGTA CTGGAATAAG |
| 1021 | CCCGAGCCAC AGCCCCCCTA CACAGCTGCT TCCGCCCAGT TCCGTCGAGC CTCCTTTATG |
| 1081 | GGCTCCACCT TCAACATCAG CCTGAACAAA GAGGAGATGG AGTTCCAGCC CAATCAGGAG |
| 1141 | GACGAGGAGG ATGCTCACGC TGGCATCATT GGCCGCTTCC TAGGCCTGCA GTCCCATGAT |
| 1201 | CACCATCCTC CCAGGGCAAA CTCAAGGACC AAACTACTGT GGCCCAAGAG GGAATCCCTT |
| 1261 | CTCCACGAGG GCCTGCCCAA AAACCACAAG GCAGCCAAAC AGAACGTTAG GGGCCAGGAA |
| 1321 | GACAACAAGG CCTGGAAGCT TAAGGCTGTG GACGCCTTCA AGTCTGCCCC ACTGTATCAG |
| 1381 | AGGCCAGGCT ACTACAGTGC CCCACAGACG CCCCTCAGCC CCACTCCCAT GTTCTTCCCC |
| 1441 | CTAGAACCAT CAGCGCCGTC AAAGCTTCAC AGTGTCACAG GCATAGACAC CAAAGACAAA |
| 1501 | AGCTTAAAGA CTGTGAGTTC TGGGGCCAAG AAAAGTTTTG AATTGCTCTC AGAGAGCGAT |
| 1561 | GGGGCCTTGA TGGAGCACCC AGAAGTATCT CAAGTGAGGA GGAAAACTGT GGAGTTTAAC |
| 1621 | CTGACGGATA TGCCAGAGAT CCCCGAAAAT CACCTCAAAG AACCTTTGGA ACAATCACCA |
| 1681 | ACCAACATAC ACACTACACT CAAAGATCAC ATGGATCCTT ATTGGGCCTT GGAAAACAGG |
| 1741 | GATGAAGCAC ATTCCTAA. |

17. (canceled)

18. The composition of claim 6, wherein the sequence encoding the polyA signal comprises a sequence isolated or derived from a mammalian Bovine Growth Hormone (BGH) gene.

19. (canceled)

20. The composition of claim 6, wherein the sequence encoding the exon comprises a sequence isolated or derived from a rabbit (Oryctolagus cuniculus) beta globin gene.

21.-22. (canceled)

23. The composition of 6, wherein the sequence encoding the intron comprises
a sequence encoding a splice donor site, and
a sequence encoding a splice branch point and acceptor site.

24. (canceled)

25. The composition of claim 23, wherein the sequence encoding the splice donor site comprises a sequence isolated or derived from a chicken (Gallus gallus) beta actin gene (CBA) or a rabbit (Oryctolagus cuniculus) beta globin gene.

26.-27. (canceled)

28. A vector comprising a composition of claim 1.

29. The vector of claim 28, wherein the vector is a plasmid.

30. (canceled)

31. The vector of claim 28, wherein the vector is a viral delivery vector, optionally wherein the delivery vector comprises a single stranded viral genome, a double-stranded viral genome, or an RNA molecule.

32.-34. (canceled)

35. The delivery vector of claim 31, wherein the delivery vector comprises a sequence isolated or derived from an adeno-associated virus (AAV) vector, optionally wherein the sequence is isolated or derived from an AAV vector of serotype AAV1, AAV2, AAV3, AAV4, AAV5, AAV6, AAV7, AAV8, AAV9, AAV10, AAV11 or any combination thereof.

36. (canceled)

37. The delivery vector of claim 35, wherein the delivery vector comprises a sequence isolated or derived from an AAV vector of serotype AAV2.

38. (canceled)

39. The delivery vector of claim 37, wherein the delivery vector comprises a sequence encoding a first inverted terminal repeat (ITR) and a second ITR isolated or derived from an AAV vector of serotype AAV2 and a sequence encoding a viral gene isolated or derived from an AAV vector of serotype AAV2.

40.-41. (canceled)

42. A pharmaceutical composition comprising:
the composition of claim 1, the vector of claim 28, or the delivery vector of claim 31; and
a pharmaceutically-acceptable carrier.

43.-44. (canceled)

45. A mammalian cell comprising the composition of claim 1, the vector of claim 28, or the delivery vector of claim 31.

46.-51. (canceled)

52. The cell of claim 51, wherein the mammalian cell is a human cell, and the human cell is a neuronal cell, a glial cell, a retinal cell, a photoreceptor cell, a rod cell, a cone cell, or a cuboidal cell of the retinal pigment epithelium (RPE).

53. (canceled)

54. The cell of claim 52, wherein the human cell is an HEK293 cell or an ARPE19 cell.

55. The cell of claim 52, wherein the human cell is isolated or derived from an RPE of a human retina.

56. (canceled)

57. A method of treating macular dystrophy in a mammalian subject in need thereof, optionally wherein the subject has a mutation in one or both copies of a BEST1 gene, the method comprising administering to the subject a therapeutically effective amount of the pharmaceutical composition of claim 42, wherein administering comprises an injection or an infusion via a subretinal, a suprachoroidal or an intravitreal route, and wherein the pharmaceutical composition optionally further comprises a TMN200 buffer.

58.-85. (canceled)
